(19) United States
(12) Patent Application Publication
(10) Pub. No.: US 2024/0082218 A1
(43) Pub. Date: Mar. 14, 2024

(54) PHARMACEUTICAL COMBINATIONS COMPRISING A KRAS G12C INHIBITOR AND USES OF A KRAS G12C INHIBITOR FOR THE TREATMENT OF CANCERS

(71) Applicant: NOVARTIS AG, Basel (CH)

(72) Inventors: Vasileios ASKOXYLAKIS, Cambridge, MA (US); Saskia Maria BRACHMANN, Basel (CH); Simona COTESTA, Basel (CH); Xiaoming CUI, East Hanover, NJ (US); Jeffrey ENGELMAN, Cambridge, MA (US); Anna FARAGO, Cambridge, MA (US); Marc GERSPACHER, Basel (CH); Diana GRAUS PORTA, Basel (CH); Catherine LEBLANC, Basel (CH); Edwige Liliane Jeanne LORTHIOIS, Basel (CH); Bo LIU, Shanghai (CN); Rainer MACHAUER, Basel (CH); Robert MAH, Basel (CH); Christophe MURA, Basel (CH); Pascal RIGOLLIER, Basel (CH); Nadine SCHNEIDER, Basel (CH); Stefan STUTZ, Basel (CH); Andrea Helga Emmi VAUPEL, Basel (CH); Nicolas WARIN, Basel (CH); Andreas WEISS, Basel (CH); Rainer WILCKEN, Basel (CH); Padmaja YERRAMILLI-RAO, Cambridge, MA (US)

(21) Appl. No.: 18/267,735

(22) PCT Filed: Dec. 20, 2021
(86) PCT No.: PCT/CN2021/139694

§ 371 Date: Jun. 15, 2023

(30) Foreign Application Priority Data
Dec. 22, 2020 (WO) PCT/CN2020/138339
Jun. 22, 2021 (WO) PCT/CN2021/101546
Aug. 30, 2021 (WO) PCT/CN2021/115387

(51) Int. Cl. A61K 31/416; A61K 9/00; A61K 31/497; A61K 39/395; A61P 35/00

(52) U.S. Cl. CPC A61K 31/416; A61K 9/0053; A61K 31/497; A61K 39/3955; A61P 35/00

(57) ABSTRACT

It relates to a pharmaceutical combination comprising a KRAS G12C inhibitor, in particular 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro [3.3] heptan-2-yl} prop-2-en-1-one (Compound A) and one or more therapeutically active agents selected from a SHP2 inhibitor (e.g. TNO155) and a PD-1 inhibitor, pharmaceutical compositions comprising the same; and methods of using such combinations and compositions in the treatment or prevention of a cancer or a tumor, in particular wherein the cancer or tumor is KRAS G12C mutated.

### SEQUENCE LISTING

The instant application contains a Sequence Listing which has been submitted electronically in ASCII format and is hereby incorporated by reference in its entirety. Said ASCII copy, created on Dec. 7, 2021, is named PAT059013-WO-PCT04_ST25 and is 49.644 bytes in size.

### FIELD OF THE INVENTION

The present invention relates to a KRAS G12C inhibitor and its uses in treating cancer, particularly KRAS G12C mutated cancer (e.g. lung cancer, non-small cell lung cancer, colorectal cancer, pancreatic cancer or a solid tumor) alone and in combination with one or two additional therapeutically active agents. The present invention relates to a pharmaceutical combination comprising (i) a KRAS G12C inhibitor, such as Compound A, or a pharmaceutically acceptable salt, solvate or hydrate thereof, and a second therapeutic agent which is a SHP2 inhibitor (such as TNO155, or a pharmaceutically acceptable salt thereof), or a PD-1 inhibitor. The present invention also relates to a triple combination comprising a KRAS G12C inhibitor, such as Compound A, or a pharmaceutically acceptable salt, solvate or hydrate thereof, and a second therapeutic agent which is a SHP2 inhibitor (such as TNO155, or a pharmaceutically acceptable salt thereof) and a PD-1 inhibitor. The present invention also relates to pharmaceutical compositions comprising the same; and methods of using such combinations and compositions in the treatment or prevention of a cancer or a solid tumor, particularly a KRAS G12C mutated cancer or a KRAS G12C mutated cancer.

### BACKGROUND

Despite the recent successes of targeted therapies and immunotherapies, some cancers, in particular, metastatic cancers, remain largely incurable.

The KRAS oncoprotein is a GTPase with an essential role as regulator of intracellular signaling pathways, such as the MAPK, PI3K and Ral pathways, which are involved in proliferation, cell survival and tumorigenesis. Oncogenic activation of KRAS occurs predominantly through missense mutations in codon 12. KRAS gain-of-function mutations are found in approximately 30% of all human cancers. KRAS G12C (KRAS glycine-to-cysteine amino acid substitution at codon 12) mutation is a specific sub-mutation, prevalent in approximately 13% of lung adenocarcinomas, 4% (3-5%) of colon adenocarcinomas and a smaller fraction of other cancer types.

In normal cells, KRAS alternates between inactive GDP-bound and active GTP-bound states. Mutations of KRAS at codon 12, such as G12C, impair GTPase-activating protein (GAP)-stimulated GTP hydrolysis. In that case, the conversion of the GTP to the GDP form of KRAS G12C is therefore very slow. Consequently, KRAS G12C shifts to the active. GTP-bound state, thus driving oncogenic signaling.

Lung cancer remains the most common cancer type worldwide and the leading cause of cancer deaths in many counties, including the United States. NSCLC accounts for about 85% of all lung cancer diagnoses. KRAS mutations are detected in approximately 25% of patients with lung adenocarcinomas (Sequist et al 2011). They are most commonly seen at codon 12, with KRAS G12C mutations being most common (40% overall) in both adenocarcinoma and squamous NSCLC (Liu et al 2020). The presence of KRAS mutations is prognostic of poor survival and has been associated with reduced responsiveness to EGFR TKI treatment.

Standard of care treatment for patients with KRAS G12C mutated NSCLC consists of platinum-based chemotherapy and immune checkpoint inhibitors. Sotorasib has recently received accelerated approval from the FDA for this indication and for adult patients who have received at least one prior systemic therapy, with further confirmatory trials currently ongoing. Immunotherapy for NSCLC with immune checkpoint inhibitors has demonstrated promise, with some NSCLC patients experiencing durable disease control for years. However, such long-term non-progressors are uncommon, and treatment strategies that can increase the proportion of patients responding to and achieving lasting remission with therapy are urgently needed.

Colorectal cancer (CRC) is the fourth most frequently diagnosed cancer and the second leading cause of cancer related death in the United States. The number of new cases of CRC was approximately 150,000 in the USA in 2019, whereas more than 300,000 patients are estimated to be diagnosed with CRC in the EU in 2020 (European Cancer Information System 2020). Despite observed improvements in the overall incidence rate of CRC, the incidence in patients younger than 50 years has been increasing in recent years (Bailey et al 2015) with the authors estimating that the incidence rates for colon and rectal cancers may increase by 90% and about 124%, respectively, for patients 20-34 years of age by 2030. Systemic therapy for metastatic CRC includes various agents used alone or in combination, including chemotherapies such as 5-fluorouracil/leucovorin, capecitabine, oxaliplatin, and irinotecan; anti-angiogenic agents such as bevacizumab and ramucirumab; anti-EGFR agents including cetuximab and panitumumab for KRAS/NRAS wild-type cancers; and immunotherapies including nivolumab and pembrolizumab. The current standard of care for disseminated metastatic colorectal cancer is largely based on chemotherapy regimens including 5-FU/LV or capecitabine-, oxaliplatin- and/or irinotecan-based regimens (NCCN Clinical Practice Guidelines www.nccn.org), whereas immune checkpoint inhibitors have shown efficacy in a subgroup of tumors with high levels of microsatellite instability (MSI-H) or mismatch-repair deficiency (dMMR) (Overman et al 2018, Overman et al 2017). Treatment of patients with wild-type RAS (KRAS/NRAS) includes the EGFR inhibitors cetuximab and panitumumab (NCCN Clinical Practice Guidelines www.nccn.org).

Despite multiple active therapies, however, metastatic CRC remains incurable. While CRCs that are deficient in mismatch repair (MSI-high) exhibit high response rates to immune checkpoint inhibitor therapy, mismatch repair proficient CRCs do not. KRAS G12C mutations are prevalent in approximately 4% of all colon adenocarcinomas. Since KRAS-mutated CRCs are typically mismatch repair proficient and are not candidates for anti-EGFR therapy, this subtype of CRC is particularly in need of improved therapies.

Tumor profiling data show that there is a subset of solid tumors other than NSCLC and CRC that harbor KRAS G12C mutations. KRAS G12C is present in approximately 1-2% of malignant solid tumors, including approximately 1% of all pancreatic cancers (Biernacka et al 2016, Zehir et al 2017). KRAS G12C mutations were also found in appendiceal cancer, small-bowel cancer, hepatobiliary cancer, bladder cancer, ovarian cancer and cancers of unknown primary site (Hassar et al, N Engl Med 2021 384; 2 185-187).

Several targeted therapies are at present in clinical testing aiming to address patients with KRAS mutations by inhibiting the RAS pathway. However, the benefit of these therapies for tumors harboring KRAS G12C mutations remains uncertain at present, as not all patients responded and, in several instances, the duration of the reported responses was short, likely due to the emergence of resistance, mediated at least in part by RAS gene mutations that disrupt inhibitor binding and reactivation of downstream pathways.

Acquired resistance to single-agent therapy eventually occurs in most patients treated with KRAS G12C inhibitors. For example, out of 38 patients included in a study with adagrasib: 27 with non small-cell lung cancer, 10 with colorectal cancer, and 1 with appendiceal cancer, putative mechanisms of resistance to adagrasib were detected in 17 patients (45% of the cohort), of whom 7 (18% of the cohort) had multiple coincident mechanisms. Acquired KRAS alterations included G12D/R/V/W, G13D, Q61H, R68S, H95D/Q/R, Y96C, and high-level amplification of the KRASG12C allele. Acquired bypass mechanisms of resistance included MET amplification; activating mutations in NRAS, BRAF, MAP2K1, and RET; oncogenic fusions involving ALK, RET, BRAF, RAF1, and FGFR3; and loss-of-function mutations in NF1 and PTEN (Awad et al, Acquired Resistance to KRASG12C Inhibition in Cancer, N Engl J Med 2021; 384:2382-93. Tanaka et al (Cancer Discov 2021; 11:1913-22) describe a novel KRAS Y96D mutation affecting the switch-II pocket, to which adagrasib and other inactive-state KRAS G12C inhibitors bind, which interfered with key protein-drug interactions and conferred resistance to these inhibitors in engineered and patient-derived KRASG12C cancer models.

Thus there is the need to provide additional KRAS inhibitors with alternative modes of binding and effective treatment to overcome resistance mechanisms that arise during treatment with KRAS inhibitors such as adagrasib or sotorasib.

### BRIEF DESCRIPTION OF THE DRAWINGS

FIG. 1: Combinations of Compound A (JDQ443) and TNO155 are synergistic in KRAS G12C-mutated NSCLC cell lines. Cell viability assays with the indicated cell lines treated for 7 days with increasing concentrations of Compound A or TNO155 as single agents and combinations thereof. The matrices indicate the percentage of growth inhibition for each treatment as compared to DMSO-treated cells. The data was processed using classical synergy models (Loewe, Bliss) and synergy scores for the JDQ443/TNO155 combination were derived for each cell line: NCI-H2122: 16.7; HCC-1171: 9.7; NCI-H1373: 6.9.

FIG. 2: Compound A alone and in combination with TNO155 shows anti-tumor efficacy in a Lu99 KRAS G12C lung carcinoma mouse xenograft models. LU99 tumor-bearing nude mice were treated orally with TNO155 (2 weeks), Compound A (4 weeks), or the combination of thereof (4 weeks) at indicated dose levels and schedules. Vehicle group was treated for 9 days. All treatments were discontinued at day 28 (vertical dotted line in FIG. 2). Tumors and body weight were monitored for 5 additional days. C. **p<0.01 at day 28 of treatment, *p<0.05 at day 33, 5 days post-treatment discontinuation, using Mann-Whitney test.

FIG. 3: A combination of Compound A with TNO155 is efficacious both when TNO155 is administered continuously (abbreviated as “cont.” in FIG. 3) and when TNO is administered two weeks on and one week off.

FIG. 4: Efficacy of a combination of Compound A and TNO155 in CRC xenograft models.

FIG. 5: Anti-tumor activity of continuous Compound A treatment in mouse models. MIA PaCa-2 (A) and NCI-H2122 (B) tumor-bearing nude mice were treated orally with Compound A at indicated dose levels and schedules for 3 weeks. *p<0.05 vs vehicle using one-way ANOVA (Dunnett's post-hoc).

FIG. 6: Potent inhibition of Compound A of KRAS G12C H95Q, a double mutant mediating resistance to adagrasib in clinical trials.

FIG. 7: Effect of Compound A (JDQ443), sotorasib (AMG510) and adagrasib (MRTX-849) on the the proliferation of KRAS G12C/H95 double mutants. Ba/F3 cells expressing the indicated FLAG-KRAS^{G12C} single or double mutants were treated with the indicated compound concentrations for 3 days and the inhibtion of proliferation was assessed by Cell titer glo viability assay. The y-axis shows the % growth of treated cells relative to day 3 treatment, the x-axis shows the log concentration in μM of the KRASG12C inhibitor.

FIG. 8: Western blot analysis of ERK phosphorylation to assess the effect of Compound A (JDQ443), sotorasib (AMG510) and adagrasib (MRTX-849) on the signaling of KRAS G12C/H95 double mutants. Ba/F3 cells expressing the indicated FLAG-KRAS^{G12C} single or double mutants were treated with the indicated compound concentrations for 30 min and the inhibtion of the MAPK pathway was assessed by probing the cell lysates for reduction of pERK by westernblot.

FIG. 9A: Anti-tumor activity in MIA PaCa-2 tumor-bearing nude mice using different dosing schedules. MIA PaCa-2, NCI-H2122 and LU99 tumor-bearing nude mice were treated orally with Compound A (JDQ443) at indicated dose levels and schedules. *p<0.05 vs vehicle using one-way ANOVA (Dunnett's post-hoc).

FIG. 9B: MIA PaCa-2 tumor-bearing nude mice were treated orally with Compound A (JDQ443) at indicated dose levels and schedules. *p<0.05 vs vehicle using one-way ANOVA (Dunnett's post-hoc The 30 mg/kg dose given in a daily schedule was found to result in better tumor growth inhibition when compared to the same dose given every three days (q3d) or the same total daily dose of 90 mg/kg q3d.

FIGS. 10A-10F: Compound A in combination with TNO155 improves single-agent activity of Compound A, while efficacy can be maintained with lower doses of either drug.

FIG. 11: Efficacy correlates with target occupancy. JDQ443 shows sustained target occupancy in vivo and has dose-dependent anti-tumor activity in mice bearing KRAS G12C mutated tumor xenografts. Adding TNO155 to Compound A achieved similar target occupancy at a third of the amount of Compound A single agent.

FIG. 12: Compound A binds under the switch II loop with a novel binding mode, exploiting unique interactions with the KRAS^{G12C} protein

### SUMMARY

The invention provides new treatment options for patients suffering from cancer (including advanced and/or metastatic cancer). Provided herein are compounds, and combinations of compounds, and their uses in methods of treating cancer including lung cancer (including NSCLC), colorectal cancer, pancreatic cancer and a solid tumor), especially when the cancer or solid tumor harbors a KRAS G12C mutation. The present invention also provides a potentially beneficial novel investigative therapy for incurable disease, especially for patients with KRAS G12C mutated tumors who have already received and failed standard of care therapy for their indication or are intolerant or ineligible to approved therapies and have therefore limited treatment options. In addition, the present invention also provides Compound A alone or in combination with one or more additional therapeutic agents for use in a method of treatment for cancer patients who have developed resistance to other therapies, such as prior treatment with other KRAS inhibitors such as adagrasib and sotorasib; more preferably prior treatment with sotorasib.

Compound A is a selective covalent irreversible inhibitor of KRAS G12C which exhibits a novel binding mode, exploiting unique interactions with KRASG12C. Compound A demonstrates potent anti-tumor activity and favorable pharmacokinetic properties in preclinical models. Compound A is orally bioavailable, achieves exposures in a range predicted to confer anti-tumor activity, and is well-tolerated. Compound A was also found to potently inhibit KRAS G12C H95Q, a double mutant mediating resistance to adagrasib in clinical trials.

The data and the Examples herein show that Compound A alone and in combination with another therapeutically active agent which is selected from a SHP2 inhibitor such as TNO155, or a pharmaceutically acceptable salt thereof, a PD-1 inhibitor, and combinations thereof may be useful in treating cancer, for example, cancers driven by KRAS G12C mutations. Targeted inhibition of KRAS G12C via Compound A may result in robust antitumor responses. In addition, Compound A may provide combination benefit in patients that have for instance acquired resistance to KRAS G12C inhibitor by reactivation of RTK-MAPK pathway bypassing KRAS G12C to signal through wild type (WT) KRAS. The combination of Compound A with a SHP2 inhibitor further increased KRAS G12C target occupancy in vivo, enhanced pre-clinical anti-tumor activity, and delayed the emergence of resistance in xenografts.

In KRAS G12C NSCLC cell lines, the combination of Compound A with the SHP2 inhibitor TNO155 results in more sustained RAS pathway inhibition than Compound A alone. In addition, this combination leads to improved tumor cell growth inhibition in several cell lines. It is also shown herein that the combination of Compound A with TNO155 significantly improved the sustainability of response and time to relapse seen with Compound A as a single agent and TNO155 as single agent.

In a KRAS G12C xenograft model of esophageal cancer, a combination of Compound A with the SHP2 inhibitor TNO155 was sufficient to convert a barely responding model to a good responder.

Compound A may induce a pro-inflammatory microenvironment that enhances the efficacy of anti-PD-1 therapies such as spartalizumab or tislelizumab. Combinations of Compound A with an anti-PD-1 inhibitor (e.g. spartalizumab or tislelizumab) may thus result in improved anti-tumor activity compared to either single agent. The improved anti-tumor activity may for example be increased efficacy and/or a more durable response.

Adding a SHP2 inhibitor such as TNO155 to Compound A plus spartalizumab or to Compound A plus tislelizumab may further decrease intracellular PD-1 signaling and lead to a less suppressive tumor microenvironment allowing for an improved immune response and better anti-tumor activity compared to single agent treatment or doublet combinations. The improved anti-tumor activity may for example be increased efficacy and/or a more durable response

The present invention therefore provides a KRAS G12C inhibitor which is 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, (Compound A), or a pharmaceutically acceptable salt thereof, for use in the treatment of cancer, as described herein.

The present invention therefore also provides a pharmaceutical combination comprising a KRAS G12C inhibitor, such as Compound A. or a pharmaceutically acceptable salt, solvate or hydrate thereof, and a second therapeutically active agent selected from a SHP2 inhibitor (such as TNO155, or a pharmaceutically acceptable salt thereof), and a PD-1 inhibitor. In addition to these combinations, the present invention also provides a triple combination consisting of Compound A, or a pharmaceutically acceptable salt, solvate or hydrate thereof, a SHP2 inhibitor (such as TNO155, or a pharmaceutically acceptable salt thereof), and a PD-1 inhibitor.

The present invention also provides a pharmaceutical combination comprising
(i) 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, having the structure

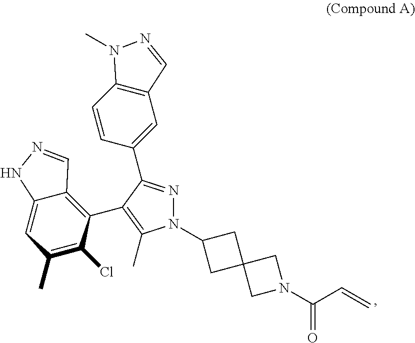

or a pharmaceutically acceptable salt thereof,
and
(ii) (3S,4S)-8-(6-amino-5-((2-amino-3-chloropyridin-4-yl)thio)pyrazin-2-yl)-3-methyl-2-oxa-8-azaspiro[4.5]decan-4-amine (TNO155), having the structure:

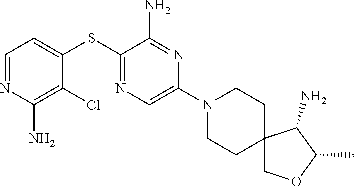 [SMILES: C[C@@H]1OCC2(CCN(c3cnc(Sc4ccnc(N)c4Cl)c(N)n3)CC2)[C@@H]1N]

or a pharmaceutically acceptable salt thereof.

The present invention also provides a pharmaceutical combination comprising:
1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt thereof and
(ii) a PD-1 inhibitor.

The present invention also provides a pharmaceutical combination comprising:
1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt thereof and
(ii) spartalizumab tislelizumab.

The present invention also provides a pharmaceutical combination comprising:
1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt thereof and
(ii) tislelizumab.

The present invention also provides a pharmaceutical combination comprising
(i) 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt thereof,
(ii) TNO155, or a pharmaceutically acceptable salt thereof,
and
(iii) a PD-1 inhibitor.

The present invention also provides a pharmaceutical combination comprising
(i) 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt thereof,
(ii) TNO155, or a pharmaceutically acceptable salt thereof,
and
(iii) spartalizumab.

The present invention also provides a pharmaceutical combination comprising
(i) 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt thereof,
(ii) TNO155, or a pharmaceutically acceptable salt thereof,
and
(iii) tislelizumab.

It will be understood that reference herein to “a combination of the invention” or “the combination(s) of the invention” is intended to include each of these pharmaceutical combinations individually and to all of these combinations as a group.

The present invention provides a combination of the invention for use in treating a cancer as described herein.

The present invention provides these pharmaceutical combinations for use in treating a cancer as described herein.

In embodiments of the invention, the PD-1 inhibitor is chosen from PDR001 (spartalizumab; Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), tislelizumab (BGB-A317; Beigene), BGB-108 (Beigene), INCSHR1210 (Incyte), or AMP-224 (Amplimmune).

In embodiments of the invention, the PD-1 inhibitor is PDR001 (spartalizumab).

In embodiments of the invention, the PD-1 inhibitor (e.g., spartalizumab) is administered at a dose of about 300-400 mg.

In embodiments of the invention, the PD-1 inhibitor (e.g., spartalizumab) is administered once every 3 weeks or once every 4 weeks.

In embodiments of the invention, the PD-1 inhibitor (e.g., spartalizumab) is administered at a dose of about 300 mg once every 3 weeks.

In embodiments of the invention, the PD-1 inhibitor (e.g., spartalizumab) is administered at a dose of about 400 mg once every 4 weeks.

In embodiments of the invention, the PD-1 inhibitor is tislelizumab.

In embodiments of the invention, the PD-1 inhibitor (e.g., tislelizumab) is administered at a dose of about 100-300 mg, or about 200-300 mg.

In embodiments of the invention, the PD-1 inhibitor (e.g., tislelizumab) is administered once every 3 weeks or once every 4 weeks.

In embodiments of the invention, the PD-1 inhibitor (e.g., tislelizumab) is administered at a dose of about 100-300 mg, once every 3 weeks.

In embodiments of the invention, the PD-1 inhibitor (e.g., tislelizumab) is administered at a dose of about 100-300 mg, once every 4 weeks.

In embodiments of the invention, the PD-1 inhibitor (e.g., tislelizumab) is administered at a dose of about 200-300 mg, once every 3 weeks.

In embodiments of the invention, the PD-1 inhibitor (e.g., tislelizumab) is administered at a dose of about 200-300 mg, once every 4 weeks. In embodiments of the invention, the PD-1 inhibitor (e.g., tislelizumab) is administered at a dose of about 200 mg once every 3 weeks.

In embodiments of the invention, the PD-1 inhibitor (e.g., tislelizumab) is administered at a dose of about 300 mg once every 4 weeks.

In another embodiment of the combination of the invention, Compound A, or a pharmaceutically acceptable salt, solvate or hydrate thereof, the SHP2 inhibitor (e.g. TNO155) or a pharmaceutically acceptable salt thereof and a PD-1 inhibitor (e.g., spartalizumab or tislelizumab) are in separate formulations.

In another embodiment, the combination of the invention is for simultaneous or sequential (in any order) administration.

In another embodiment is a method for treating or preventing cancer in a subject in need thereof comprising administering to the subject a therapeutically effective amount of the combination of the invention.

In embodiments of the invention, the cancer or tumor to be treated is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation. Cancers of unknown primary site but showing a KRAS G12C mutation may also benefit from treatment with the methods of the invention.

Suitably the cancer to be treated is lung cancer, colorectal cancer or esophageal cancer.

In embodiments of the methods of the invention, the cancer is selected from non-small cell lung cancer, colorectal cancer, pancreatic cancer and a solid tumor.

In a further embodiment of the methods, the cancer is non-small cell lung cancer.

In a further embodiment of the methods, the cancer is small cell lung cancer.

In a further embodiment of the methods, the cancer is colorectal cancer (including colorectal adenocarcinoma).

In a further embodiment of the methods, the cancer is pancreatic cancer (including pancreatic adenocarcinoma).

In a further embodiment of the methods, the cancer is uterine cancer (including uterine endometrial cancer).

In a further embodiment of the methods, the cancer is rectal cancer (including rectal adenocarcinoma).

In a further embodiment of the methods, the cancer is appendiceal cancer.

In a further embodiment of the methods, the cancer is small-bowel cancer.

In a further embodiment of the methods, the cancer is esophageal cancer.

In a further embodiment of the methods, the cancer is hepatobiliary cancer (including liver cancer and bile duct carcinoma).

In a further embodiment of the methods, the cancer is bladder cancer.

In a further embodiment of the methods, the cancer ovarian cancer.

In a further embodiment of the methods, the cancer is a solid tumor In a further embodiment of the methods, the cancer is gastric cancer.

In a further embodiment of the methods, the cancer is nasopharyngeal cancer.

In a further embodiment of the methods, the cancer is hepatocellular cancer.

In a further embodiment of the methods, the cancer is urothelial bladder cancer.

In a further embodiment of the methods, the cancer is Hodgkin's Lymphoma.

In a further embodiment, the invention provides a combination of the invention for use in the manufacture of a medicament for treating a cancer selected from: non-small cell lung cancer, colorectal cancer, pancreatic cancer and a solid tumor, optionally wherein the cancer or solid tumor is KRAS G12C mutated. In another embodiment is a pharmaceutical composition comprising the combination of the invention.

In a further embodiment, the pharmaceutical composition further comprises one or more pharmaceutically acceptable excipients as described herein.

### DESCRIPTION

KRAS G12C inhibitor Compound A

Compound A is 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one. Compound A is also known by the name “a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”.

The synthesis of Compound A and crystalline forms thereof are as described in the Examples below. Crystalline forms of Compound A, e.g., as described in the Examples, are also particularly useful in the methods and uses of the present invention.

The structure of Compound A is as follows:

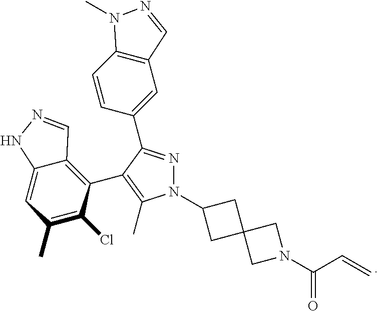

Alternatively, the structure of Compound A may be drawn as follows:

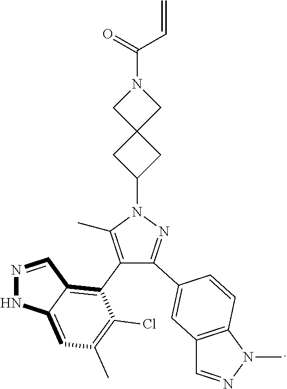

Compound A is also known as “JDQ443” or “NVP-JDQ443” and is described in Example 1 of PCT application WO2021/124222, published 24 Jun. 2021. WO2021/124222 which is incorporated by reference in its entirety also describes crystalline forms (e.g. Modification HA), useful in the combinations, uses and methods of the present invention.

Compound A binds under the switch II loop of KRAS with a novel binding mode, exploiting unique interactions with the KRASG12C protein compared to sotorasib and adagrasib. Compound A potently inhibits KRASG12C cellular signaling and proliferation in a mutated selective manner by irreversibly trapping the GDP-bound state of KRASG12C through formation of a covalent bond with cysteine at position 12.

Compound A shows sustained target occupancy (TO) in vivo (KRASG12C TO t1/2˜66 h in the MiaPaCa2 model) despite a blood half-life of ˜2 hours and exhibits a linear PK/PD (pharmacokinetic/pharmacodynamic modeling) relationship. Compound A has dose-dependent anti-tumor activity in mice bearing KRAS G12C mutated tumor xenografts comparable to sotorasib and adagrasib (FIG. 5). In mouse, rat, and dog, Compound A is orally bioavailable, achieves exposures in a range predicted to confer anti-tumor activity, and is well-tolerated. Continuous delivery of Compound A using mini-pump administration demonstrates that area under the curve (AUC), rather than maximal concentration (Cmax), is the driver of efficacy.

Other KRAS G12C inhibitors useful in combinations and methods of the invention includes a compound selected from 1-(4-(6-chloro-8-fluoro-7-(3-hydroxy-5-vinylphenyl)quinazolin-4-yl)piperazin-1-yl)prop-2-en-1-one-,-methane (1/2); (S)-1-(4-(6-chloro-8-fluoro-7-(2-fluoro-6-hydroxyphenyl)quinazolin-4-yl)piperazin-1-yl)prop-2-en-1-one; and 2-((S)-1-acryloyl-4-(2-(((S)-1-methylpyrrolidin-2-yl)methoxy)-7-(naphthalen-1-yl)-5,6,7,8-tetrahydropyrido[3,4-d]pyrimidin-4-yl)piperazin-2-yl)acetonitrile and the compounds detailed in WO2013/155223, WO2014/143659, WO2014/152588, WO2014/160200, WO2015/054572, WO2016/044772, WO2016/049524, WO2016164675, WO2016168540, WO2017/058805, WO2017015562, WO2017058728, WO2017058768, WO2017058792, WO2017058805, WO2017058807, WO2017058902, WO2017058915, WO2017087528, WO2017100546, WO2017/201161, WO2018/064510, WO2018/068017, WO2018/119183, WO2018/217651, WO2018/140512, WO2018/140513, WO2018/140514, WO2018/140598, WO2018/140599, WO2018/140600, WO2018/143315, WO2018/206539, WO2018/218069, WO2018/218070, WO2018/218071, WO2019/051291, WO2019/099524, WO2019/110751, WO2019/141250, WO2019/150305, WO2019/155399, WO2019/213516, WO2019/213526, WO2019/215203, WO2019/217307 and WO2019/217691, WO2019/232419, WO2020/028706, WO2020/047192, EP3628664, WO2020081282, WO2020085504, WO2020/085493. WO2020/097537, WO2020/106640, WO2020/113071, WO2020/146613, WO2020/156285, WO2020/181110, WO2020/178282, WO2020/216190. WO2020/236940, WO2020/233592, WO2020/238791, WO2020/239077, WO2020/239123, WO2020/259513, WO2020/259573, WO2020/259432, WO2021/000885, WO2021/023154, WO2021/027943, WO2021/027911, CN112390796, WO2021/037018, CN112430234, CN112442029, WO2021/043322, WO2021/055728.

### SHP2 Inhibitors

Examples of SHP2 inhibitors useful in combinations and methods of the present invention include TNO155, JAB3068 (Jacobio), JAB3312 (Jacobio), RLY1971 (Roche), SAR442720 (Sanofi), RMC4450 (Revolution Medicines), BBP398 (Navire), BR790 (Shanghai Blueray), SH3809 (Nanjing Sanhome), PF0724982 (Pfizer), ERAS601 (Erasca), RX-SHP2 (Redx Pharma), ICP189 (InnoCare), HBI2376 (HUYA Bioscience), ETS001 (Shanghai ETERN Biopharma), TAS-ASTX (Taiho Oncology) and X-37-SHP2 (X-37).

A particularly preferred SHP2 inhibitor for use according to the invention is (3S,4S)-8-(6-amino-5-((2-amino-3-chloropyridin-4-yl)thio)pyrazin-2-yl)-3-methyl-2-oxa-8-azaspiro[4.5]decan-4-amine (TNO155), or a pharmaceutically acceptable salt thereof. TNO155 is synthesized according to example 69 of WO2015/107495, which is incorporated by reference in its entirety. A preferred salt of TNO155 is the succinate salt.

In addition, SHP2 inhibitors include compounds described in WO2015/107493, WO2015/107494, WO2015/107495, WO2016/203406, WO2016/203404, WO2016/203405, WO2017/216706, WO2017/156397, WO2020/063760, WO2018/172984, WO2017/211303, WO21/061706, WO2019/183367, WO2019/183364, WO2019/165073, WO2019/067843, WO2018/218133, WO2018/081091. WO2018/057884, WO2020/247643, WO2020/076723, WO2019/199792, WO2019/118909. WO2019/075265, WO2019/051084, WO2018/136265. WO2018/136264, WO2018/013597, WO2020/033828, WO2019/213318, WO2019/158019, WO2021/088945, WO2020/081848, WO21/018287, WO2020/094018, WO2021/033153, WO2020/022323, WO2020/177653, WO2021/073439, WO2020/156243, WO2020/156242, WO2020/249079, WO2020/033286, WO2021/061515, WO2019/182960, WO2020/094104, WO2020/210384, WO2020/181283, WO2021/043077, WO2021/028362, WO2020/259679, WO2020/108590 & WO2019/051469.

TNO155 is an orally bioavailable, allosteric inhibitor of Src homology-2 domain containing protein tyrosine phsophatase-2 (SHP2, encoded by the PTPN11 gene), which transduces signals from activated receptor tyrosine kinases (RTKs) to downstream pathways, including the mitogen-activated protein kinase (MAPK) pathway. SHP2 has also been implicated in immune checkpoint and cytokine receptor signaling. TNO155 has demonstrated efficacy in a wide range of RTK-dependent human cancer cell lines and in vivo tumor xenografts.

### PD1-Inhibitors

The Programmed Death 1 (PD-1) protein is an inhibitory member of the extended CD28/CTLA-4 family of T cell regulators. Two ligands for PD-1 have been identified, PD-L1 (B7-H1) and PD-L2 (B7-DC), that have been shown to downregulate T cell activation upon binding to PD-1. PD-L1 is abundant in a variety of human cancers.

PD-1 is known as an immunoinhibitory protein that negatively regulates TCR signals. The interaction between PD-1 and PD-L1 can act as an immune checkpoint, which can lead to, for example, a decrease in tumor infiltrating lymphocytes, a decrease in T-cell receptor mediated proliferation, and/or immune evasion by cancerous cells. Immune suppression can be reversed by inhibiting the local interaction of PD-1 with PD-L1 or PD-L2; the effect is additive when the interaction of PD-1 with PD-L2 is blocked as well.

Pharmaceutical combinations of the invention comprising a PD1-inhibitor (e.g., spartalizumab or tislelizumab) may be particularly useful in the methods of the invention as KRAS G12C is associated with a higher rate of PD-L1 expression.

In certain embodiments, Compound A, or a pharmaceutically acceptable salt, solvate or hydrate thereof, is further administered in combination with a PD-1 inhibitor. In certain embodiments, Compound A, or a pharmaceutically acceptable salt, solvate or hydrate thereof, and a SHP2 inhibitor (e.g. TNO155, or a pharmaceutically acceptable salt thereof), is further administered in combination with a PD-1 inhibitor. In some embodiments, the PD-1 inhibitor is chosen from spartalizumab (PDR001, Novartis), Nivolumab (Bristol-Myers Squibb), Pembrolizumab (Merck & Co), Pidilizumab (CureTech), MEDI0680 (Medimmune), cemiplimab (REGN2810) (Regeneron), TSR-042 (Tesaro), PF-06801591 (Pfizer), tislelizumab (BGD-A317, Beigene), BGB-108 (Beigene), INCSHR1210 (Incyte), or AMP-224 (Amplimmune). A particularly preferred PD-1 inhibitor for use according to the invention is spartalizumab. Another particularly preferred PD-1 inhibitor for use according to the invention is tislelizumab.

PDR00 is also known as spartalizumab, an anti-PD-1 antibody molecule described in US 2015/0210769, published on Jul. 30, 2015, entitled “Antibody Molecules to PD-1 and Uses Thereof,” incorporated by reference in its entirety.

Tislelizumab is also known as BGB-A317, an anti-PD-1 antibody described in WO2015035606, published on 19 March 2015, which is incorporated by reference in its entirety.

Further anti-PD-1 antibody molecules include the following: Nivolumab (Bristol-Myers Squibb), also known as MDX-1106, MDX-1106-04, ONO-4538, BMS-936558, or OPDIVO*. Nivolumab (clone 5C4) and other anti-PD-1 antibodies are disclosed in U.S. Pat. No. 8,008,449 and WO 2006/121168, incorporated by reference in their entirety; Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 13444, U.S. Pat. No. 8,354,509, and WO 2009/114335, incorporated by reference in their entirety;

Pidilizumab (CureTech), also known as CT-01. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety;

MEDI0680 (Medimmune), also known as AMP-514. MEDI0680 and other anti-PD-1 antibodies are disclosed in U.S. Pat. No. 9,205,148 and WO 2012/145493, incorporated by reference in their entirety; AMP-224 (B7-DCIg (Amplimmune), e.g., disclosed in WO 2010/027827 and WO 2011/066342, incorporated by reference in their entirety;
REGN2810 (Regeneron); PF-06801591 (Pfizer); BGB-A317 or BGB-108 (Beigene); INCSHR1210 (Incyte), also known as INCSHR01210 or SHR-1210; TSR-042 (Tesaro), also known as ANB011; and further known anti-PD-1 antibodies including those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In one embodiment, the anti-PD-1 inhibitor is spartalizumab, also known as PDR001. In some embodiments, the anti-PD-1 antibody molecule is BAP049-Clone E or BAP049-Clone B. In other embodiments, the anti-PD1 antibody molecule is tislelizumab, also known as BGB-A317.

In embodiments of the invention, the anti-PD-1 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of BAP049-Clone-E or BAP049-Clone-B disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In some embodiments, the CDRs are according to the combined CDR definitions of both Kabat and Chothia (e.g., as set out in Table 1). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In embodiments of the invention, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

In embodiments of the invention, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 1.

In embodiments of the invention, the anti-PD-1 antibody molecule comprises a heavy chain variable region (VH) comprising a VHCDR1 amino acid sequence of SEQ ID NO: 547, a VHCDR2 amino acid sequence of SEQ ID NO: 548, and a VHCDR3 amino acid sequence of SEQ ID NO: 549; and a light chain variable region (VL) comprising a VLCDR1 amino acid sequence of SEQ ID NO: 542, a VLCDR2 amino acid sequence of SEQ ID NO: 543, and VLCDR3 amino acid sequence of SEQ ID NO: 544, each disclosed in Table 1. In embodiments of the invention, the anti-PD-1 antibody molecule comprises a VH comprising the amino acid sequence of SEQ ID NO: 550, or an amino acid sequence at least 85%, 90%, 95%, or 99% identical or higher to SEQ ID NO: 550. In one embodiment, the anti-PD-1 antibody molecule comprises a VL comprising the amino acid sequence of SEQ ID NO: 545, or an amino acid sequence at least 85%, 90%, 95%, or 99% identical or higher to SEQ ID NO: 545. In embodiments of the invention, the anti-PD-1 antibody molecule comprises a heavy chain comprising the amino acid sequence of SEQ ID NO: 551, or an amino acid sequence at least 85%, 90%, 95%, or 99% identical or higher to SEQ ID NO: 551. In one embodiment, the anti-PD-1 antibody molecule comprises a light chain comprising the amino acid sequence of SEQ ID NO: 546, or an amino acid sequence at least 85%, 90%, 95%, or 99% identical or higher to SEQ ID NO: 546.

In embodiments of the invention, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520.

In embodiments of the invention, the anti-PD-1 antibody molecule comprises a VH comprising the amino acid sequence of SEQ ID NO: 506 and a VL comprising the amino acid sequence of SEQ ID NO: 516.

In embodiments of the invention, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In embodiments of the invention, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 WO 2022/135346 PCT/CN2021/13%94 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In embodiments of the invention, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

The antibody molecules described herein can be made as described in WO2015035606, published on 19 March 2015, which is incorporated by reference in its entirety.

In certain embodiments, a combined inhibition of a checkpoint inhibitor (e.g., an inhibitor of TIM-3 described herein) with a TGF-β inhibitor is further combined with a PD-1 inhibitor and used to treat a cancer (e.g., a myelofibrosis).

In some embodiments, the PD-1 inhibitor (e.g., spartalizumab) is administered at a dose between about 100 mg to about 600 mg. e.g., about 100 mg to about 500 mg, about 100 mg to about 400 mg, about 100 mg to about 300 mg, about 100 mg to about 200 mg, about 200 mg to about 600 mg, about 200 mg to about 500 mg, about 200 mg to about 400 mg, about 200 mg to about 300 mg, about 300 mg to about 600 mg, about 300 mg to about 500 mg, about 300 mg to about 400 mg, about 400 mg to about 600 mg, about 400 mg to about 500 mg, or about 500 mg to about 600 mg. In some embodiments, the PD-1 inhibitor (e.g., spartalizumab) is administered at a dose of about 100 mg, about 200 mg, about 300 mg, about 400 mg, about 500 mg, or about 600 mg. In some embodiments, the PD-1 inhibitor (e.g., spartalizumab) is administered once every four weeks. In some embodiments, (e.g., spartalizumab) is administered once every three weeks. In some embodiments, (e.g., spartalizumab) is administered intravenously. In some embodiments, (e.g., spartalizumab) is administered over a period of about 20 minutes to 40 minutes (e.g., about 30 minutes).

In some embodiments, the PD-1 inhibitor (e.g., spartalizumab) is administered at a dose between about 300 mg to about 500 mg (e.g., about 400 mg), intravenously, over a period of about 20 minutes to about 40 minutes (e.g., about 30 minutes), once every two weeks. In some embodiments, the PD-1 inhibitor (e.g., spartalizumab) is administered at a dose between about 200 mg to about 400 mg (e.g., about 300 mg), intravenously, over a period of about 20 minutes to about 40 minutes (e.g., about 30 minutes), once every three weeks.

In some embodiments, the PD-1 inhibitor (e.g., spartalizumab) is administered in combination with a TIM-3 inhibitor (e.g., an anti-TIM3 antibody) and a TGF-β inhibitor (e.g., NIS793).

In embodiments of the invention, the PD-1 inhibitor (e.g., tislelizumab) is administered once every 3 weeks or once every 4 weeks.

In embodiments of the invention, the PD-1 inhibitor (e.g., tislelizumab) is administered at a dose of about 100-300 mg, once every 3 weeks.

In embodiments of the invention, the PD-1 inhibitor (e.g., tislelizumab) is administered at a dose of about 100-300 mg, once every 4 weeks.

In embodiments of the invention, the PD-1 inhibitor (e.g., tislelizumab) is administered at a dose of about 200-300 mg, once every 3 weeks.

In embodiments of the invention, the PD-1 inhibitor (e.g., tislelizumab) is administered at a dose of about 200-300 mg, once every 4 weeks.

In embodiments of the invention, the PD-1 inhibitor (e.g., tislelizumab) is administered at a dose of about 200 mg once every 3 weeks.

In embodiments of the invention, the PD-1 inhibitor (e.g., tislelizumab) is administered at a dose of about 300 mg once every 4 weeks.

TABLE 1
Amino acid and nucleotide sequences of exemplary
anti-PD-1 antibody molecules

| | | |
|---|---|---|
| BAP049-Clone-B | | |
| HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMH |
| | | WVRQATGQGLEWMGNIYPGTGGSNFDEKFKNRVTI |
| | | TADKSTSTAYMELSSLRSEDTAVYYCTRWTTGTGA |
| | | YWGQGTTVTVSS |
| SEQ ID NO: 507 | DNA VH | GAGGTGCAGCTGGTGCAGTCAGGCGCCGAAGTGAA |
| | | GAAGCCCGGCGAGTCACTGAGAATTAGCTGTAAAG |
| | | GTTCAGGCTACACCTTCACTACCTACTGGATGCAC |
| | | TGGGTCCGCCAGGCTACCGGTCAAGGCCTCGAGTG |
| | | GATGGGTAATATCTACCCCGGCACCGGCGGCTCTA |
| | | ACTTCGACGAGAAGTTTAAGAATAGAGTGACTATC |
| | | ACCGCCGATAAGTCTACTAGCACCGCCTATATGGA |
| | | ACTGTCTAGCCTGAGATCAGAGGACACCGCCGTCT |
| | | ACTACTGCACTAGGTGGACTACCGGCACAGGCGCC |
| | | TACTGGGGTCAAGGCACTACCGTGACCGTGTCTAG |
| | | C |
| SEQ ID NO: 508 | Heavy | EVQLVQSGAEVKKPGESLRISCKGSGYTFTTYWMH |
| | chain | WVRQATGQGLEWMGNIYPGTGGSNFDEKFKNRVTI |
| | | TADKSTSTAYMELSSLRSEDTAVYYCTRWTTGTGA |
| | | YWGQGTTVTVSSASTKGPSVFPLAPCSRSTSESTA |
| | | ALGCLVKDYFPEPVTVSWNSGALTSGVHTFPAVLQ |
| | | SSGLYSLSSVVTVPSSSLGTKTYTCNVDHKPSNTK |
| | | VDKRVESKYGPPCPPCPAPEFLGGPSVFLFPPKPK |
| | | DTLMISRTPEVTCVVVDVSQEDPEVQFNWYVDGVE |
| | | VHNAKTKPREEQFNSTYRVVSVLTVLHQDWLNGKE |
| | | YKCKVSNKGLPSSIEKTISKAKGQPREPQVYTLPP |
| | | SQEEMTKNQVSLTCLVKGFYPSDIAVEWESNGQPE |
| | | NNYKTTPPVLDSDGSFFLYSRLTVDKSRWQEGNVF |
| | | SCSVMHEALHNHYTQKSLSLSLG |
| SEQ ID NO: 509 | DNA | GAGGTGCAGCTGGTGCAGTCAGGCGCCGAAGTGAA |
| | heavy | GAAGCCCGGCGAGTCACTGAGAATTAGCTGTAAAG |
| | chain | GTTCAGGCTACACCTTCACTACCTACTGGATGCAC |
| | | TGGGTCCGCCAGGCTACCGGTCAAGGCCTCGAGTG |
| | | GATGGGTAATATCTACCCCGGCACCGGCGGCTCTA |
| | | ACTTCGACGAGAAGTTTAAGAATAGAGTGACTATC |
| | | ACCGCCGATAAGTCTACTAGCACCGCCTATATGGA |
| | | ACTGTCTAGCCTGAGATCAGAGGACACCGCCGTCT |
| | | ACTACTGCACTAGGTGGACTACCGGCACAGGCGCC |
| | | TACTGGGGTCAAGGCACTACCGTGACCGTGTCTAG |
| | | CGCTAGCACTAAGGGCCCGTCCGTGTTCCCCCTGG |
| | | CACCTTGTAGCCGGAGCACTAGCGAATCCACCGCT |
| | | GCCCTCGGCTGCCTGGTCAAGGATTACTTCCCGGA |
| | | GCCCGTGACCGTGTCCTGGAACAGCGGAGCCCTGA |
| | | CCTCCGGAGTGCACACCTTCCCCGCTGTGCTGCAG |
| | | AGCTCCGGGCTGTACTCGCTGTCGTCGGTGGTCAC |
| | | GGTGCCTTCATCTAGCCTGGGTACCAAGACCTACA |
| | | CTTGCAACGTGGACCACAAGCCTTCCAACACTAAG |
| | | GTGGACAAGCGCGTCGAATCGAAGTACGGCCCACC |
| | | GTGCCCGCCTTGTCCCGCGCCGGAGTTCCTCGGCG |
| | | GTCCCTCGGTCTTTCTGTTCCCACCGAAGCCCAAG |
| | | GACACTTTGATGATTTCCCGCACCCCTGAAGTGAC |
| | | ATGCGTGGTCGTGGACGTGTCACAGGAAGATCCGG |
| | | AGGTGCAGTTCAATTGGTACGTGGATGGCGTCGAG |
| | | GTGCACAACGCCAAAACCAAGCCGAGGGAGGAGCA |
| | | GTTCAACTCCACTTACCGCGTCGTGTCCGTGCTGA |
| | | CGGTGCTGCATCAGGACTGGCTGAACGGGAAGGAG |
| | | TACAAGTGCAAAGTGTCCAACAAGGGACTTCCTAG |
| | | CTCAATCGAAAAGACCATCTCGAAAGCCAAGGGAC |
| | | AGCCCCGGGAACCCCAAGTGTATACCCTGCCACCG |
| | | AGCCAGGAAGAAATGACTAAGAACCAAGTCTCATT |
| | | GACTTGCCTTGTGAAGGGCTTCTACCCATCGGATA |
| | | TCGCCGTGGAATGGGAGTCCAACGGCCAGCCGGAA |
| | | AACAACTACAAGACCACCCCTCCGGTGCTGGACTC |
| | | AGACGGATCCTTCTTCCTCTACTCGCGGCTGACCG |
| | | TGGATAAGAGCAGATGGCAGGAGGGAAATGTGTTC |
| | | AGCTGTTCTGTGATGCATGAAGCCCTGCACAACCA |
| | | CTACACTCAGAAGTCCCTGTCCCTCTCCCTGGGA |
| BAP049-Clone-B | | |
| LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQ |
| | | KNFLTWYQQKPGKAPKLLIYWASTRESGVPSRFSG |
| | | SGSGTDFTFTISSLQPEDIATYYCONDYSYPYTFG |
| | | QGTKVEIK |
| SEQ ID NO: 517 | DNA VL | GAGATCGTCCTGACTCAGTCACCCGCTACCCTGAG |
| | | CCTGAGCCCTGGCGAGCGGGCTACACTGAGCTGTA |
| | | AATCTAGTCAGTCACTGCTGGATAGCGGTAATCAG |
| | | AAGAACTTCCTGACCTGGTATCAGCAGAAGCCCGG |
| | | TAAAGCCCCTAAGCTGCTGATCTACTGGGCCTCTA |
| | | CTAGAGAATCAGGCGTGCCCTCTAGGTTTAGCGGT |
| | | AGCGGTAGTGGCACCGACTTCACCTTCACTATCTC |
| | | TAGCCTGCAGCCCGAGGATATCGCTACCTACTACT |
| | | GTCAGAACGACTATAGCTACCCCTACACCTTCGGT |
| | | CAAGGCACTAAGGTCGAGATTAAG |
| SEQ ID NO: 518 | Light | EIVLTQSPATLSLSPGERATLSCKSSQSLLDSGNQ |
| | chain | KNFLTWYQQKPGKAPKLLIYWASTRESGVPSRFSG |
| | | SGSGTDFTFTISSLQPEDIATYYCONDYSYPYTFG |
| | | QGTKVEIKRTVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSENRGEC |
| SEQ ID NO: 519 | DNA | GAGATCGTCCTGACTCAGTCACCCGCTACCCTGAG |
| | light | CCTGAGCCCTGGCGAGCGGGCTACACTGAGCTGTA |
| | chain | AATCTAGTCAGTCACTGCTGGATAGCGGTAATCAG |
| | | AAGAACTTCCTGACCTGGTATCAGCAGAAGCCCGG |
| | | TAAAGCCCCTAAGCTGCTGATCTACTGGGCCTCTA |
| | | CTAGAGAATCAGGCGTGCCCTCTAGGTTTAGCGGT |
| | | AGCGGTAGTGGCACCGACTTCACCTTCACTATCTC |
| | | TAGCCTGCAGCCCGAGGATATCGCTACCTACTACT |
| | | GTCAGAACGACTATAGCTACCCCTACACCTTCGGT |
| | | CAAGGCACTAAGGTCGAGATTAAGCGTACGGTGGC |
| | | CGCTCCCAGCGTGTTCATCTTCCCCCCCAGCGACG |
| | | AGCAGCTGAAGAGCGGCACCGCCAGCGTGGTGTGC |
| | | CTGCTGAACAACTTCTACCCCCGGGAGGCCAAGGT |
| | | GCAGTGGAAGGTGGACAACGCCCTGCAGAGCGGCA |
| | | ACAGCCAGGAGAGCGTCACCGAGCAGGACAGCAAG |
| | | GACTCCACCTACAGCCTGAGCAGCACCCTGACCCT |
| | | GAGCAAGGCCGACTACGAGAAGCATAAGGTGTACG |
| | | CCTGCGAGGTGACCCACCAGGGCCTGTCCAGCCCC |
| | | GTGACCAAGAGCTTCAACAGGGGCGAGTGC |
| BAP049-Clone-E | | |
| HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMH |
| | | WVRQATGQGLEWMGNIYPGTGGSNFDEKFKNRVTI |
| | | TADKSTSTAYMELSSLRSEDTAVYYCTRWTTGTGA |
| | | YWGQGTTVTVSS |
| SEQ ID NO: 507 | DNA VH | GAGGTGCAGCTGGTGCAGTCAGGCGCCGAAGTGAA |
| | | GAAGCCCGGCGAGTCACTGAGAATTAGCTGTAAAG |
| | | GTTCAGGCTACACCTTCACTACCTACTGGATGCAC |
| | | TGGGTCCGCCAGGCTACCGGTCAAGGCCTCGAGTG |
| | | GATGGGTAATATCTACCCCGGCACCGGCGGCTCTA |
| | | ACTTCGACGAGAAGTTTAAGAATAGAGTGACTATC |
| | | ACCGCCGATAAGTCTACTAGCACCGCCTATATGGA |
| | | ACTGTCTAGCCTGAGATCAGAGGACACCGCCGTCT |
| | | ACTACTGCACTAGGTGGACTACCGGCACAGGCGCC |
| | | TACTGGGGTCAAGGCACTACCGTGACCGTGTCTAG |
| | | C |
| SEQ ID NO: 508 | Heavy | EVQLVQSGAEVKKPGESLRISCKGSGYTFTTYWMH |
| | chain | WVRQATGQGLEWMGNIYPGTGGSNFDEKFKNRVTI |
| | | TADKSTSTAYMELSSLRSEDTAVYYCTRWTTGTGA |
| | | YWGQGTTVTVSSASTKGPSVFPLAPCSRSTSESTA |
| | | ALGCLVKDYFPEPVTVSWNSGALTSGVHTFPAVLQ |
| | | SSGLYSLSSVVTVPSSSLGTKTYTCNVDHKPSNTK |
| | | VDKRVESKYGPPCPPCPAPEFLGGPSVFLFPPKPK |
| | | DTLMISRTPEVTCVVVDVSQEDPEVQFNWYVDGVE |
| | | VHNAKTKPREEQFNSTYRVVSVLTVLHQDWLNGKE |
| | | YKCKVSNKGLPSSIEKTISKAKGQPREPQVYTLPP |
| | | SQEEMTKNQVSLTCLVKGFYPSDIAVEWESNGQPE |
| | | NNYKTTPPVLDSDGSFFLYSRLTVDKSRWQEGNVF |
| | | SCSVMHEALHNHYTQKSLSLSLG |
| SEQ ID NO: 509 | DNA | GAGGTGCAGCTGGTGCAGTCAGGCGCCGAAGTGAA |
| | heavy | GAAGCCCGGCGAGTCACTGAGAATTAGCTGTAAAG |
| | chain | GTTCAGGCTACACCTTCACTACCTACTGGATGCAC |
| | | TGGGTCCGCCAGGCTACCGGTCAAGGCCTCGAGTG |
| | | GATGGGTAATATCTACCCCGGCACCGGCGGCTCTA |
| | | ACTTCGACGAGAAGTTTAAGAATAGAGTGACTATC |
| | | ACCGCCGATAAGTCTACTAGCACCGCCTATATGGA |
| | | ACTGTCTAGCCTGAGATCAGAGGACACCGCCGTCT |
| | | ACTACTGCACTAGGTGGACTACCGGCACAGGCGCC |
| | | TACTGGGGTCAAGGCACTACCGTGACCGTGTCTAG |
| | | CGCTAGCACTAAGGGCCCGTCCGTGTTCCCCCTGG |
| | | CACCTTGTAGCCGGAGCACTAGCGAATCCACCGCT |
| | | GCCCTCGGCTGCCTGGTCAAGGATTACTTCCCGGA |
| | | GCCCGTGACCGTGTCCTGGAACAGCGGAGCCCTGA |
| | | CCTCCGGAGTGCACACCTTCCCCGCTGTGCTGCAG |
| | | AGCTCCGGGCTGTACTCGCTGTCGTCGGTGGTCAC |
| | | GGTGCCTTCATCTAGCCTGGGTACCAAGACCTACA |
| | | CTTGCAACGTGGACCACAAGCCTTCCAACACTAAG |
| | | GTGGACAAGCGCGTCGAATCGAAGTACGGCCCACC |
| | | GTGCCCGCCTTGTCCCGCGCCGGAGTTCCTCGGCG |
| | | GTCCCTCGGTCTTTCTGTTCCCACCGAAGCCCAAG |
| | | GACACTTTGATGATTTCCCGCACCCCTGAAGTGAC |
| | | ATGCGTGGTCGTGGACGTGTCACAGGAAGATCCGG |
| | | AGGTGCAGTTCAATTGGTACGTGGATGGCGTCGAG |
| | | GTGCACAACGCCAAAACCAAGCCGAGGGAGGAGCA |
| | | GTTCAACTCCACTTACCGCGTCGTGTCCGTGCTGA |
| | | CGGTGCTGCATCAGGACTGGCTGAACGGGAAGGAG |
| | | TACAAGTGCAAAGTGTCCAACAAGGGACTTCCTAG |
| | | CTCAATCGAAAAGACCATCTCGAAAGCCAAGGGAC |
| | | AGCCCCGGGAACCCCAAGTGTATACCCTGCCACCG |
| | | AGCCAGGAAGAAATGACTAAGAACCAAGTCTCATT |
| | | GACTTGCCTTGTGAAGGGCTTCTACCCATCGGATA |
| | | TCGCCGTGGAATGGGAGTCCAACGGCCAGCCGGAA |
| | | AACAACTACAAGACCACCCCTCCGGTGCTGGACTC |
| | | AGACGGATCCTTCTTCCTCTACTCGCGGCTGACCG |
| | | TGGATAAGAGCAGATGGCAGGAGGGAAATGTGTTC |
| | | AGCTGTTCTGTGATGCATGAAGCCCTGCACAACCA |
| | | CTACACTCAGAAGTCCCTGTCCCTCTCOCTGGGA |
| BAP049-Clone-E | | |
| LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQ |
| | | KNFLTWYQQKPGQAPRLLIYWASTRESGVPSRFSG |
| | | SGSGTDFTFTISSLEAEDAATYYCONDYSYPYTFG |
| | | QGTKVEIK |
| SEQ ID NO: 521 | DNA VL | GAGATCGTCCTGACTCAGTCACCCGCTACCCTGAG |
| | | CCTGAGCCCTGGCGAGCGGGCTACACTGAGCTGTA |
| | | AATCTAGTCAGTCACTGCTGGATAGCGGTAATCAG |
| | | AAGAACTTCCTGACCTGGTATCAGCAGAAGCCCGG |
| | | TCAAGCCCCTAGACTGCTGATCTACTGGGCCTCTA |
| | | CTAGAGAATCAGGCGTGCCCTCTAGGTTTAGCGGT |
| | | AGCGGTAGTGGCACCGACTTCACCTTCACTATCTC |
| | | TAGCCTGGAAGCCGAGGACGCCGCTACCTACTACT |
| | | GTCAGAACGACTATAGCTACCCCTACACCTTCGGT |
| | | CAAGGCACTAAGGTCGAGATTAAG |
| SEQ ID NO: 522 | Light | EIVLTQSPATLSLSPGERATLSCKSSQSLLDSGNQ |
| | chain | KNFLTWYQQKPGQAPRLLIYWASTRESGVPSRFSG |
| | | SGSGTDFTFTISSLEAEDAATYYCONDYSYPYTFG |
| | | QGTKVEIKRTVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| SEQ ID NO: 523 | DNA | GAGATCGTCCTGACTCAGTCACCCGCTACCCTGAG |
| | light | CCTGAGCCCTGGCGAGCGGGCTACACTGAGCTGTA |
| | chain | AATCTAGTCAGTCACTGCTGGATAGCGGTAATCAG |
| | | AAGAACTTCCTGACCTGGTATCAGCAGAAGCCCGG |
| | | TCAAGCCCCTAGACTGCTGATCTACTGGGCCTCTA |
| | | CTAGAGAATCAGGCGTGCCCTCTAGGTTTAGCGGT |
| | | AGCGGTAGTGGCACCGACTTCACCTTCACTATCTC |
| | | TAGCCTGGAAGCCGAGGACGCCGCTACCTACTACT |
| | | GTCAGAACGACTATAGCTACCCCTACACCTTCGGT |
| | | CAAGGCACTAAGGTCGAGATTAAGCGTACGGTGGC |
| | | CGCTCCCAGCGTGTTCATCTTCCCCCCCAGCGACG |
| | | AGCAGCTGAAGAGCGGCACCGCCAGCGTGGTGTGC |
| | | CTGCTGAACAACTTCTACCCCCGGGAGGCCAAGGT |
| | | GCAGTGGAAGGTGGACAACGCCCTGCAGAGCGGCA |
| | | ACAGCCAGGAGAGCGTCACCGAGCAGGACAGCAAG |
| | | GACTCCACCTACAGCCTGAGCAGCACCCTGACCCT |
| | | GAGCAAGGCCGACTACGAGAAGCATAAGGTGTACG |
| | | CCTGCGAGGTGACCCACCAGGGCCTGTCCAGCCCC |
| | | GTGACCAAGAGCTTCAACAGGGGCGAGTGC |
| BAP049-Clone-B | | |
| HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGA |
| (Kabat) | | CGAGAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-B | | |
| LC | | |
| SEQ ID NO: 529 | LCDR1 | AAATCTAGTCAGTCACTGCTGGATAGCGGTAATCA |
| (Kabat) | | GAAGAACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAA |
| (Chothia) | | CTTC |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E | | |
| HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGA |
| (Kabat) | | CGAGAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E | | |
| LC | | |
| SEQ ID NO: 529 | | AAATCTAGTCAGTCACTGCTGGATAGCGGTAATCA |
| (Kabat) | LCDR1 | GAAGAACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAA |
| (Chothia) | | CTTC |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BGB-A317 | | |
| SEQ ID NO: 542 | LCDR1 | KSSESVSNDVA |
| SEQ ID NO: 543 | LCDR2 | YAFHRFT |
| SEQ ID NO: 544 | LCDR3 | HQAYSSPYT |
| SEQ ID NO: 545 | VL | DIVMTQSPDSLAVSLGERATINCKSSESVSNDVAW |
| | | YQQKPGQPPKLLINYAFHRFTGVPDRFSGSGYGTD |
| | | FTLTISSLQAEDVAVYYCHQAYSSPYTFGQGTKLE |
| | | IK |
| SEQ ID NO: 546 | Light | DIVMTQSPDSLAVSLGERATINCKSSESVSNDVAW |
| | chain | YQQKPGQPPKLLINYAFHRFTGVPDRFSGSGYGTD |
| | | FTLTISSLQAEDVAVYYCHQAYSSPYTFGQGTKLE |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| SEQ ID NO: 547 | HCDR1 | GFSLTSYGVH |
| SEQ ID NO: 548 | HCDR2 | VIYADGSTNYNPSLKS |
| SEQ ID NO: 549 | HCDR3 | ARAYGNYWYIDV |
| SEQ ID NO: 550 | VH | QVQLQESGPGLVKPSETLSLTCTVSGFSLTSYGVH |
| | | WIRQPPGKGLEWIGVIYADGSTNYNPSLKSRVTIS |
| | | KDTSKNQVSLKLSSVTAADTAVYYCARAYGNYWYI |
| | | DVWGQGTTVTVSS |
| SEQ ID NO: 551 | Heavy | QVQLQESGPGLVKPSETLSLTCTVSGFSLTSYGVH |
| | chain | WIRQPPGKGLEWIGVIYADGSTNYNPSLKSRVTIS |
| | | KDTSKNQVSLKLSSVTAADTAVYYCARAYGNYWYI |
| | | DVWGQGTTVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNT |
| | | KVDKRVESKYGPPCPPCPAPPVAGGPSVFLFPPKP |
| | | KDTLMISRTPEVTCVVVAVSQEDPEVQFNWYVDGV |
| | | EVHNAKTKPREEQFNSTYRVVSVLTVVHQDWLNGK |
| | | EYKCKVSNKGLPSSIEKTISKAKGQPREPQVYTLP |
| | | PSQEEMTKNQVSLTCLVKGFYPSDIAVEWESNGQP |
| | | ENNYKTTPPVLDSDGSFFLYSKLTVDKSRWQEGNV |
| | | FSCSVMHEALHNHYTQKSLSLSLGK |

In combinations and methods of the invention, each of the therapeutically active agents can be administered separately, simultaneously, or sequentially, in any order.

In combinations and methods of the invention, Compound A and/or TNO155 may be administered in an oral dose form.

In combinations and methods of the invention, tislelizumab may be administered intravenously.

In another embodiment, there is provided a pharmaceutical composition comprising a pharmaceutical combination of the invention and at least one pharmaceutically acceptable carrier.

### KRAS G12C Inhibitors

The methods and combinations of the invention may be particularly useful for treating a cancer or tumor which is resistant to prior treatment with another KRAS G12C inhibitor. Examples of such a KRAS G12C inhibitor include sotorasib (Amgen), adagrasib (Mirati), D-1553 (InventisBio), B11701963 (Boehringer), GDC6036 (Roche), JNJ74699157 (J&J), X-Chem KRAS (X-Chem), LY3537982 (Lilly), BI1823911 (Boehringer), AS KRAS G12C (Ascentage Pharma), SF KRAS G12C (Sanofi), RMC032 (Revolution Medicine), JAB-21822 (Jacobio Pharmaceuticals), AST-KRAS G12C (Allist Pharmaceuticals), AZ KRAS G12C (Astra Zeneca), NYU-12VC1 (New York University), and RMC6291 (Revolution Medicines).

In one embodiment, the cancer or tumor to be treated is resistant to or has progressed on prior treatment with sotorasib or adagrasib.

In one embodiment, the cancer. e.g. NSCLC, has previously been treated with a KRAS G12C inhibitor (e.g. sotorasib, adagrasib, D-1553, and GDC6036).

It is expected that a combination therapy which involves Compound A and (i) a SHP2 inhibitor or a (ii) a PD-1 inhibitor, or which involves Compound A and both a SHP2 inhibitor and a PD-1 inhibitor would be particularly useful in overcoming this resistance.

### Cancers to be Treated

Compound A is a potent and selective covalent inhibitor of KRAS G12C that binds to KRAS G12C and traps it into an inactive guanosine diphosphate (GDP)-bound state. By inhibiting KRAS G12C in tumor cells and preventing downstream signaling, Compound A has the potential to reduce tumor growth in patients with KRAS G12C mutated cancers or tumors.

As Compound A binds with KRAS G12 with alternative modes of binding and has shown activity against a double mutant which has been identified as mediating resistance to adagrasib in clinical trials (see Tanaka 2021), it may provide effective treatment alone and in combination with one or two agents selected from a SHP2 inhibitor (e,g, TNO 155) and a PD-1 inhibitor (e,g, spartalizumab or tislelizumab) to overcome resistance mechanisms that arise during treatment with KRAS inhibitors such as adagrasib or sotorasib.

Compound A and combinations comprising Compound A may thus be useful in the treatment of cancer and in cancers or tumors which are KRAS G12C mutated. Compound A and combinations of the invention may be useful in the treatment of a cancer or tumor which is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation. Cancers of unknown primary site but showing a KRAS G12C mutation may also benefit from treatment with the methods of the invention.

Other cancers to be treated by the compounds, combinations and methods of the invention include gastric cancer, nasopharyngeal cancer, hepatocellular cancer, and Hodgkin's Lymphoma, particularly when the cancer harbors a KRAS G12C mutation.

In particular, the present invention provides methods of treating and combinations for use in treating a cancer which is selected from the group consisting of lung cancer (such as lung adenocarcinoma and non-small cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma) and a solid tumor, particularly when the cancer or tumor harbors a KRAS G12C mutation.

The cancer may be at an early, intermediate, late stage or may be metastatic cancer. In some embodiments, the cancer is an advanced cancer. In some embodiments, the cancer is a metastatic cancer. In some embodiments, the cancer is a relapsed cancer. In some embodiments, the cancer is a refractory cancer. In some embodiments, the cancer is a recurrent cancer. In some embodiments, the cancer is an unresectable cancer.

The cancer may be at an early, intermediate, late stage or metastatic cancer.

Compound A and combinations of the invention may also be useful in the treatment of solid malignancies characterized by mutations of RAS.

Compound A and combinations of the invention may also be useful in the treatment of solid malignancies characterized by one or more mutations of KRAS, in particular G12C mutations in KRAS.

The present invention provides Compound A and combinations of the invention for use in the treatment of a cancer or solid tumor characterized by an acquired KRAS alteration which is selected from G12D/R/V/W, G13D, Q61H, R68S, H95D/Q/R, Y96C, Y96 D and high-level amplification of the KRASG12C allele, or characterized by an acquired bypass mechanisms of resistance, These bypass mechanisms of resistance include MET amplification; activating mutations in NRAS, BRAF, MAP2K1, and RET; oncogenic fusions involving ALK, RET, BRAF, RAF1, and FGFR3; and loss-of-function mutations in NF1 and PTEN.

Thus, as a further embodiment, the present invention provides Compound A, or a pharmaceutically acceptable salt, solvate or hydrate thereof, alone or in combination with a second therapeutic agent which is selected from a SHP2 inhibitor such as TNO155, or a pharmaceutically acceptable salt thereof, and a PD-1 inhibitor for use in therapy. The present invention also provides a triple combination consisting of Compound A, or a pharmaceutically acceptable salt, solvate or hydrate thereof, a SHP2 inhibitor such as TNO155, or a pharmaceutically acceptable salt thereof, and a PD-1 inhibitor. As a further embodiment, the present invention provides a combination of the invention for use in therapy. In a preferred embodiment, the therapy or the therapy which the medicament is useful for is selected from a disease which may be treated by inhibition of RAS mutated proteins, in particular, KRAS, HRAS or NRAS G12C mutated proteins. In another embodiment, the invention provides a method of treating a disease, which is treated by inhibition of a RAS mutated protein, in particular, a G12C mutated of either KRAS, HRAS or NRAS protein, in a subject in need thereof, wherein the method comprises the administration of a therapeutically effective amount of a compound of the invention, or a combination of the invention, to the subject.

In a more preferred embodiment, the disease is selected from the afore-mentioned list, suitably non-small cell lung cancer, colorectal cancer and pancreatic cancer.

In a preferred embodiment, the therapy is for a disease, which may be treated by inhibition of a RAS mutated protein, in particular, a G12C mutated of either KRAS, HRAS or NRAS protein. In a more preferred embodiment, the disease is selected from the afore-mentioned list, suitably non-small cell lung cancer, colorectal cancer and pancreatic cancer, which is characterized by a G12C mutation in either KRAS, HRAS or NRAS.

In another embodiment is method of treating (e.g., one or more of reducing, inhibiting, or delaying progression) a cancer or a tumor in a subject comprising administering to a subject in need thereof a pharmaceutical composition comprising Compound A, or pharmaceutically acceptable salt thereof, in combination with a second therapeutic agent as described herein.

The present invention therefore provides a method of treating (e.g., one or more of reducing, inhibiting, or delaying progression) cancer or tumor in a patient in need thereof, wherein the method comprises administering to the patient in need thereof, a therapeutically active amount of Compound A, or a pharmaceutically acceptable salt, solvate or hydrate thereof, as a single agent or as combination therapy with a therapeutically active amount of one or two therapeutically active agents selected from a SHP2 inhibitor (e.g., TNO155, or a pharmaceutically acceptable salt thereof), and a PD-1 inhibitor (e.g., spartalizumab or tislelizumab), wherein the cancer is lung cancer (including lung adenocarcinoma and non-small cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma) and a solid tumor, optionally wherein the cancer is KRAS-, NRAS- or HRAS-G12C mutated.

In embodiments of the invention, the cancer or tumor to be treated is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, particularly when the cancer or tumor harbors a KRAS G12C mutation. Cancers of unknown primary site but showing a KRAS G12C mutation may also benefit from treatment with the methods of the invention.

In embodiments of the methods of the invention, the cancer is selected from non-small cell lung cancer, colorectal cancer, pancreatic cancer and a solid tumor.

In a further embodiment of the methods, the cancer is colorectal cancer.

In a further embodiment of the methods, the cancer is gastric cancer.

In a further embodiment of the methods, the cancer is nasopharyngeal cancer.

In a further embodiment of the methods, the cancer is non-small cell lung cancer.

In a further embodiment of the methods, the cancer is small cell lung cancer.

In a further embodiment of the methods, the cancer is pancreatic cancer.

In a further embodiment of the methods, the cancer is a solid tumor.

In a further embodiment of the methods, the cancer is appendiceal cancer.

In a further embodiment of the methods, the cancer is small-bowel cancer.

In a further embodiment of the methods, the cancer is esophageal cancer.

In a further embodiment of the methods, the cancer is hepatobiliary cancer.

In a further embodiment of the methods, the cancer is hepatocellular cancer.

In a further embodiment of the methods, the cancer is bladder cancer.

In a further embodiment of the methods, the cancer is urothelial bladder cancer.

In a further embodiment of the methods, the cancer is ovarian cancer.

In a further embodiment of the methods, the cancer is Hodgkin's Lymphoma.

Compound A and the methods and combinations of the invention may be useful as first line therapy.

Compound A and the methods and combinations of the invention may also be useful as first line therapy.

The methods and combinations of the invention may be useful as second line of therapy or as more advanced lines of therapy. In this respect, the unique interactions of Compound A with mutated KRAS G12C, e.g. compared to other KRASG12C inhibitors such as sotorasib or adagrasib would be useful in targeting resistance mutations which may arise after treatment with other KRAS G12C inhibitors such as sotorasib or adagrasib.

Compound A, alone or in combination with one or more therapeutic agent as described herein, may be useful to treat a cancer or a tumor as described herein, wherein the patient may be a treatment agnostic patient or a patient who has progressed and/or relapsed on previous therapy.

Previous therapy includes:
radiotherapy;
surgery;
standard of care therapy, such as fluropyrimidine-, oxaliplatin-, and/or irinotecan-based chemotherapy;
chemotherapy, such as pemetrexed, or platinum-based chemotherapy;
immune checkpoint inhibitor therapy, such as anti-PD-1 immunotherapy (e.g. pembrolizumab, spartalizumab, tislelizumab or nivolumab) or anti-PD-L1 immunotherapy (e.g. atezolizumab or durvalumab);
therapy with a KRAS G12C inhibitor (for example sotorasib, adagrasib, GDC6036, or D-1553, suitably sotorasib or adagrasib; more particularly sotorasib).

Previous therapy also includes pembrolizumab alone or in combination with chemotherapy. For example, the patient or subject to be treated by the methods and combinations of the invention include a patient suffering from cancer, e.g. KRAS G12C mutated NSCLC (including advanced (metastatic or unresectable) KRAS G12C mutated NSCLC), optionally wherein the patient has received and progressed on previous therapy.

In embodiments of the invention, including methods of treatment with Compound A as monotherapy and in combinations as described herein, the subject or patient to be treated is selected from:
a patient suffering from a KRAS G12C mutated solid tumor (e.g. advanced (metastatic or unresectable) KRAS G12C mutated solid tumor), optionally wherein the patient has received and failed standard of care therapy or is intolerant or ineligible or refractive to previous investigative and/or approved therapies;
a patient suffering from KRAS G12C mutated NSCLC (e.g., advanced (metastatic or unresectable) KRAS G12C mutated NSCLC), optionally wherein the patient who has received and failed a platinum-based chemotherapy regimen and an immune checkpoint inhibitor therapy either in combination or in sequence;
a patient suffering from KRAS G12C mutated CRC (e.g., advanced (metastatic or unresectable) KRAS G12C mutated CRC), optionally wherein the patient has received and failed standard of care therapy, including a fluropyrimidine-, oxaliplatin-, and/or irinotecan-based chemotherapy; and
a patient suffering from KRAS G12C mutated NSCLC (e.g., advanced (metastatic or unresectable) KRAS G/2C mutated NSCLC), optionally wherein the patient who has previously been treated with a KRAS G12C inhibitor (e.g. sotorasib, adagrasib, GDC6036 or D-1553);
a patient suffering from KRAS G12C mutated cancer, optionally wherein the patient has previously been treated with another KRAS G12C inhibitor (e.g. sotorasib, adagrasib, GDC6036 or D-1553);
a patient suffering from a KRAS G12C mutated cancer, optionally wherein the patient has previously been treated with another KRAS G12C inhibitor (e.g. sotorasib, adagrasib, GDC6036 or D-1553) and the patient is suffering from KRAS G12C mutated cancer selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, optionally wherein the patient has previously been treated with a KRAS G12C inhibitor (e.g. sotorasib, adagrasib, GDC6036 or D-1553);
a patient suffering from lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor;
a patient suffering from a KRAS G12C mutated cancer selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, optionally wherein the patient has previously been treated with a KRAS G12C inhibitor (e.g. sotorasib, adagrasib, GDC6036 or D-1553);
a patient suffering from lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor;
a treatment-naive patient with locally advanced or metastatic NSCLC harboring KRASG12C mutations.

In one embodiment, there is provided Compound A, or a pharmaceutically acceptable salt thereof for use in the treatment of KRAS G12C mutant NSCLC in a patient who has previously been treated with a KRAS G12C inhibitor such as sotorasib or adagrasib.

In one embodiment, there is provided a pharmaceutical combination comprising Compound A, or a pharmaceutically acceptable salt thereof, and TNO, or a pharmaceutically acceptable salt thereof, for use in the treatment of KRAS G12C mutant NSCLC in a patient who has previously been treated with a KRAS G12C inhibitor such as sotorasib or adagrasib.

In one embodiment, there is provided Compound A, or a pharmaceutically acceptable salt thereof for use in the treatment of KRAS G12C mutant NSCLC in a patient who has previously received chemotherapy and/or immunotherapy, followed by a KRAS G12C inhibitor such as sotorasib or adagrasib.

In one embodiment, there is provided a pharmaceutical combination comprising Compound A, or a pharmaceutically acceptable salt thereof, and TNO, or a pharmaceutically acceptable salt thereof, for use in the treatment of KRAS G12C mutant NSCLC in a patient who has previously received chemotherapy and/or immunotherapy, followed by a KRAS G12C inhibitor such as sotorasib or adagrasib.

In a further embodiment, the Compound A, or pharmaceutically acceptable salt thereof, administered to the subject in need thereof in an amount which is effective to treat the cancer.

In embodiments of the invention, the amounts of Compound A, or pharmaceutically acceptable salt thereof and the second therapeutic agent-and the third therapeutic agent, if present—are administered to the subject in need thereof in amounts which are effective to treat the cancer.

In a further embodiment, the second therapeutic agent or third therapeutic agent is TNO155, or a pharmaceutically acceptable salt thereof.

In a further embodiment, the second therapeutic agent or third therapeutic agent is an immunomodulator, such as a PD-1 inhibitor.

In a further embodiment, the PD-1 inhibitor is selected from PDR001, Nivolumab, Pembrolizumab, Pidilizumab, MEDI0680, REGN2810, TSR-042, PF-06801591, BGB-A317, BGB-108, INCSHR1210, or AMP-224.

In a further embodiment, the PD-1 inhibitor is PDR001 (spartazilumab).

In a further embodiment, the PD-1 inhibitor is BGB-A317 (tislelizumab).

### Dosages and Dosing Regimens

Preclinical models were utilized to predict the efficacious exposure for Compound A. Doses of Compound A when used alone or in combination therapy according to the present invention are designed to be pharmacologically active and result in an anti-tumor response.

Dose selection for the SHP2 inhibitor and/or the PD-1 inhibitor is likewise guided by a mixture of pharmacokinetics (PK), pharmacodynamics, safety, and efficacy data.

In embodiments of the invention, Compound A, or a pharmaceutically acceptable salt, solvate or hydrate thereof, is administered at a therapeutically effective dose ranging from 50 to 1600 mg per day, e.g. from 200 to 1600 mg per day, or from 400 to 1600 mg per day. The total daily dose of Compound A may be selected from 50, 100, 150, 200, 250, 300, 350, 400, 450, 500, 600, 800, 1000, 1200 and 1600 mg. For example, the total daily dose of Compound A may be selected from 200, 300, 400, 600, 800, 1000, 1200 and 1600 mg.

In preferred embodiments of the invention, Compound A, or a pharmaceutically acceptable salt, solvate or hydrate thereof, is administered at a therapeutically effective dose ranging from 100 to 400 mg per day, e.g. from 200 to 400 mg per day. For example, the total daily dose of Compound A may be selected from 100 mg, 200 mg, 400 mg and 600 mg; more preferably from 100 mg, 200 mg, and 400 mg.

The total daily dose of Compound A may be administered continuously, on a QD (once a day) or BID (twice a day) regimen.

For example, Compound A may be administered at a dose of 200 mg BID (total daily dose of 400 mg), 400 mg QD (total daily dose 400 mg). Compound A may also be administered at a dose of 100 mg BID (total daily dose of 200 mg) or 200 mg QD (total daily dose 200 mg). Compound A may be also administered at a total daily dose of 600 mg daily, preferably administered twice daily (i.e. 300 mg BID).

Preclinical target occupancy models coupled with PK patient data predict that a BID scheduling may lead to an increased response in a larger number of patients. Suitably, Compound A may be administered at a dose of 100 mg BID or at a dose of 200 mg BID or at a dose of 300 mg BID. Typically, Compound A may be administered at a dose of 100 mg administered twice a day (total daily dose of 200 mg) or at a dose of 200 mg administered twice a day (total daily dose of 400 mg).

Doses of TNO 155 in the combinations of the present invention are designed to be pharmacologically active and have a potential for a synergistic anti-tumor effect while at the same time minimizing the possibility of unacceptable toxicity due to suppressive activities by both agents on MAPK pathway signaling. TNO may be administered continuously or intermittently, e.g. a 2 weeks on/1 week off schedule, to maintain clinical efficacy and minimize clinical adverse effects. Thus TNO155 may be administered at a total daily dose ranging from 10 to 80 mg, or from 10 to 60 mg. For example, the total daily dose of TNO155 may be selected from 10, 15, 20, 30, 40, 60 and 80 mg. The total daily dose of TNO155 may be administered continuously, QD (once a day) or BID (twice a day) on QD or BID on a 2 weeks on/I week off schedule. The total daily dose of TNO155 may be administered continuously, QD (once a day) or BID (twice a day) on QD or BID on continuously (i.e. without a drug holiday).

In combinations of the invention, Compound A is administered at a dose ranging from 50 to 1600 mg per day (e.g., 50, 100, 150, 200, 250, 300, 350, 400, 450, 500, 600, 800, 1000, 1200 or 1600 mg) or from 200 to 1600 mg per day (e.g., 200, 300, 400, 600, 800, 1000, 1200 or 1600 mg) and TNO155 is administered at a dose ranging from 10 to 80 mg per day (0, 15, 20, 30, 40, 60 or 80 mg), wherein Compound A is administered on a continuous schedule and TNO is administered either on a two week on/one week off schedule or on a continuous schedule.

In combinations of the invention, spartalizumab is administered at a dose of about 300 mg once every 3 weeks, or at a dose of about 400 mg once every 4 weeks. More preferably, spartalizumab is administered at a dose of about 300 mg once every 3 weeks (Q3W), by injection (e.g., subcutaneously or intravenously).

In combinations of the invention, Compound A is administered on a continuous schedule at a dose ranging from 50 to 1600 mg per day (e.g., 50, 100, 150, 200, 250, 300, 350, 400, 450, 500, 600, 800, 1000, 1200 or 1600 mg) or from 200 to 1600 mg per day (e.g., 200, 300, 400, 600, 800, 1000, 1200 or 1600 mg) and spartalizumab is administered at a dose of about 300 mg once every 3 weeks, or at a dose of about 400 mg once every 4 weeks.

In combinations of the invention, Compound A is administered on a continuous schedule at a dose ranging from 50 to 1600 mg per day (e.g., 50, 100, 150, 200, 250, 300, 350, 400, 450, 500, 600, 800, 1000, 1200 or 1600 mg) or from 200 to 1600 mg per day (e.g., 200, 300, 400, 600, 800, 1000, 1200 or 1600 mg), TNO155 is administered either on a two week on/one week off schedule or on a continuous schedule at a dose ranging from 10 to 80 mg (0, 15, 20, 30, 40, 60 or 80 mg), and spartalizumab is administered at a dose of about 300 mg once every 3 weeks or at a dose of about 400 mg once every 4 weeks.

In combinations of the invention, tislelizumab is administered at a dose of about 200 mg once every 3 weeks, or at a dose of about 300 mg once every 4 weeks. Tislelizumab may be administered by injection (e.g., subcutaneously or intravenously).

In combinations of the invention, Compound A is administered on a continuous schedule at a dose ranging from 50 to 1600 mg per day (e.g., 50, 100, 150, 200, 250, 300, 350, 400, 450, 500, 600, 800, 1000, 1200 or 1600 mg) and tislelizumab is administered at a dose of about 200 mg once every 3 weeks, or at a dose of about 300 mg once every 4 weeks.

In combinations of the invention, Compound A is administered on a continuous schedule at a dose ranging from 50 to 1600 mg per day (e.g., 50, 100, 150, 200, 250, 300, 350, 400, 450, 500, 600, 800, 1000, 1200 or 1600 mg), TNO155 is administered either on a two week on/one week off schedule or on a continuous schedule at a dose ranging from 10 to 80 mg (0, 15, 20, 30, 40, 60 or 80 mg), and tislelizumab is administered at a dose of about 200 mg once every 3 weeks, or at a dose of about 300 mg once every 4 weeks.

Exemplary dosages and doses of the combinations are as follows.

TABLE

| Compound A- | |
|---|---|
| continuous schedule* | TNO155 dose, 2 weeks on/1 week off |
| 50 mg BID | 10 mg or 20 mg |
| 100 mg QD | 10 mg or 20 mg |
| 100 mg BID | 10 mg or 20 mg |
| 200 mg QD | 10 mg or 20 mg |
| 200 mg BID | 10 mg or 20 mg |
| 400 mg QD | 10 mg or 20 mg |
| 300 mg BID | 10 mg or 20 mg |

*when tislelizumab is used in combination with Compound A, or in combination with Compound A and TNO155, tislelizumab is preferentially administered at a dose of about 200 mg once every 3 weeks.

Or as follows:

| Compound A- continuous schedule* | TNO155 dose, continuous schedule |
|---|---|
| 50 mg BID | 10 mg or 20 mg |
| 100 mg QD | 10 mg or 20 mg |
| 100 mg BID | 10 mg or 20 mg |
| 200 mg QD | 10 mg or 20 mg |
| 200 mg BID | 10 mg or 20 mg |
| 400 mg QD | 10 mg or 20 mg |
| 300 mg BID | 10 mg or 20 mg |

*when tislelizumab is used in combination with Compound A, or in combination with Compound A and TNO155, tislelizumab is preferentially administered at a dose of about 200 mg once every 3 weeks.

In combinations of the invention, tislelizumab is administered at a dose of about 200 mg once every 3 weeks, and TNO is administered at a total daily dose of 10 mg to 60 mg, administered once or twice daily, (preferably on a two week on/one week off schedule). Suitably, Compound A may be administered at a dose of 100 mg-300 mg BID, preferably 100-200 mg BID, e.g. 100 mg BID or at a dose of 200 mg BID or at a dose of 300 mg BID.

### Pharmaceutical Compositions

Compound A, or a pharmaceutically acceptable salt, solvate or hydrate thereof, may be administered either simultaneously with, or before or after, one or more (e.g., one or two) other therapeutic agents. Compound A, or a pharmaceutically acceptable salt, solvate or hydrate thereof, may be administered separately, by the same or different route of administration, or together in the same pharmaceutical composition as the other agents.

In another aspect, the present invention provides pharmaceutically acceptable compositions which comprise a therapeutically effective amount of one or more (e.g., one or two) therapeutic agents selected from Compound A, TNO155 and a PD-1 inhibitor, formulated together with one or more pharmaceutically acceptable carriers (additives) and/or diluents.

In another aspect, the present invention provides a pharmaceutical composition comprising a compound of the present invention, or a pharmaceutically acceptable salt thereof, and a pharmaceutically acceptable carrier. In another aspect, the present invention provides a pharmaceutical composition comprising a KRAS G12C inhibitor, such as Compound A, or a pharmaceutically acceptable salt, solvate or hydrate thereof, and one or more (e.g., one or two) therapeutically active agents selected from a SHP2 inhibitor such as TNO155, or a pharmaceutically acceptable salt thereof and a PD-1 inhibitor. In a further embodiment, the composition comprises at least two pharmaceutically acceptable carriers, such as those described herein. For purposes of the present invention, unless designated otherwise, solvates and hydrates are generally considered compositions. Preferably, pharmaceutically acceptable carriers are sterile. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.

In an embodiment, the pharmaceutical compositions are capsules comprising the active ingredient only.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs, solutions or solid dispersion. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulation well-known in the art. Such may contain solubilizers, stabilizers, tonicity enhancing agents, buffers and preservatives.

As used herein, a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

In one embodiment, the invention provides a product comprising Compound A, or a pharmaceutically acceptable salt, solvate or hydrate thereof, and at least one other therapeutic agent as a combined preparation for simultaneous, separate or sequential use in therapy. In one embodiment, the therapy is the treatment of a disease or condition characterized by a KRAS, HRAS or NRAS G12C mutation. Products provided as a combined preparation include a composition comprising the compound of the present invention and one or more (e.g., one or two) therapeutically active agents selected from a SHP2 inhibitor such as TNO155, or a pharmaceutically acceptable salt thereof and a PD-1 inhibitor together in the same pharmaceutical composition, or Compound A, or a pharmaceutically acceptable salt, solvate or hydrate, thereof, and the other therapeutic agent(s) in separate form, e.g. in the form of a kit.

In one embodiment, the invention provides a pharmaceutical composition comprising a compound of the present invention and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains Compound A, or a pharmaceutically acceptable salt, solvate or hydrate thereof; TNO155, or a pharmaceutically acceptable salt thereof, and a PD-1 inhibitor (e.g., spartalizumab or tislelizumab). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent. The compound of the present invention may be administered either simultaneously with, or before or after, one or more other therapeutic agent. The compound of the present invention may be administered separately, by the same or different route of administration, or together in the same pharmaceutical composition as the other agents.

In general, a suitable daily dose of the combination of the invention will be that amount of each compound which is the lowest dose effective to produce a therapeutic effect.

In another aspect, the present invention provides pharmaceutically acceptable compositions which comprise a therapeutically effective amount of one or more of the subject compounds, as described above, formulated together with one or more pharmaceutically acceptable carriers (additives) and/or diluents.

### Definitions

The general terms used hereinbefore and hereinafter preferably have within the context of this disclosure the following meanings, unless otherwise indicated, where more general terms whereever used may, independently of each other, be replaced by more specific definitions or remain, thus defining more detailed embodiments of the invention.

The term “KRAS G12C mutated cancer” is to be understood as being equivalent to the term “KRAS G12C mutant cancer”. The terms “KRAS G12C mutated NSCLC” and the like are to be construed accordingly. Whether a cancer is KRAS G12C mutated or not can be determined by tests known in the art, e.g. by an FDA approved test.

Where a dosage is mentioned as ‘about’ a particular value or mentioned as a particular value (i.e. without the term “about” preceding that particular value, it is intended to include a range around the specified value of plus or minus 10%, or plus or minus 5%. As is customary in the art, dosages refer to the amount of the therapeutic agent in its free form. For example, when a dosage of 20 mg of TNO155 is referred to, and TNO155 is used as its succinate salt, the amount of the therapeutic agent used is equivalent to 20 mg of the free form of TNO155.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The term “treating” or “treatment” as used herein comprises a treatment relieving, reducing or alleviating at least one symptom in a subject or effecting a delay of progression of a disease. For example, treatment can be the diminishment of one or several symptoms of a disorder or partial or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “combination therapy” or “in combination with” refers to the administration of two or more therapeutic agents to treat a condition or disorder described in the present disclosure (e.g., cancer). Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. Synergistic effect, as used herein, refers to action of two therapeutic agents such as, for example, a compound TNO155 as a SHP2 inhibitor and Compound A, producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As set out above, certain embodiments of the present compounds may contain a basic functional group, such as amino or alkylamino, and are, thus, capable of forming pharmaceutically acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. The pharmaceutically acceptable salt of TNO155, for example, is succinate.

In the combination of the invention, Compound A, TNO155 and a PD-1 inhibitor, is also intended to represent unlabeled forms as well as isotopically labeled forms of the compounds. Isotopically labeled compounds have one or more atoms replaced by an atom having a selected atomic mass or mass number. Examples of isotopes that can be incorporated into TNO155 and a PD-1 inhibitor include isotopes, where possible, of hydrogen, carbon, nitrogen, oxygen, and chlorine, for example, ²H, ³H, ¹¹C, ¹³C, ¹⁴C, ¹⁵N, ³⁵S, ³⁶Cl. The invention includes isotopically labeled TNO155 and a PD-1 inhibitor, for example into which radioactive isotopes, such as ³H and ¹⁴C, or non-radioactive isotopes, such as ²H and ¹³C, are present. Isotopically labelled TNO155 and a PD-1 inhibitor are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds of the invention can generally be prepared by conventional techniques known to those skilled in the art or by processes analogous to those described in the accompanying Examples using appropriate isotopically-labeled reagents.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of either Compound A, TNO155 or a PD-1 inhibitor. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in TNO155 or a PD-1 inhibitor is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In Compound A, a methyl group, e.g. on the indazolyl ring, may be deuterated or perdeuterated.

### EXAMPLES

### Example 1: Preparation of 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A)

A synthesis of 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A) is as described below.

Compound A is also known by the name “a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one”.

### General Methods and Conditions:

Temperatures are given in degrees Celsius. If not mentioned otherwise, all evaporations are performed under reduced pressure, typically between about 15 mm Hg and 100 mm Hg (=20-133 mbar).

Abbreviations used are those conventional in the art.

Mass spectra were acquired on LC-MS, SFC-MS, or GC-MS systems using electrospray, chemical and electron impact ionization methods with a range of instruments of the following configurations: Waters Acquity UPLC with Waters SQ detector or Mass spectra were acquired on LCMS systems using ESI method with a range of instruments of the following configurations: Waters Acquity LCMS with PDA detector. [M+H]⁺ refers to the protonated molecular ion of the chemical species.

NMR spectra were run with Bruker Ultrashield™400 (400 MHz), Bruker Ultrashield™600 (600 MHz) and Bruker Ascend™400 (400 MHz) spectrometers, both with and without tetramethylsilane as an internal standard. Chemical shifts (6-values) are reported in ppm downfield from tetramethylsilane, spectra splitting pattern are designated as singlet (s), doublet (d), triplet (t), quartet (q), multiplet, unresolved or more overlapping signals (m), broad signal (br). Solvents are given in parentheses. Only signals of protons that are observed and not overlapping with solvent peaks are reported.

Celite: Celite® (the Celite corporation)=filtering aid based on diatomaceous earth Phase separator: Biotage—Isolute phase separator—(Part number: 120-1908-F for 70 mL and part number: 120-1909-J for 150 mL)

SiliaMetS®Thiol: SiliCYCLE thiol metal scavenger—(R51030B, Particle Size: 40-63 μm).

X-ray powder diffraction (XRPD) patterns described herein were obtained using a Bruker Advance D8 in reflection geometry. Powder samples were analyzed using a zero background Si flat sample holder. The radiation was Cu Kα (λ=1.5418 Å). Patterns were measured between 2° and 40° 2theta.

Sample amount: 5-10 mg
Sample holder: zero background Si flat sample holder

### XRPD Parameters:

| | |
|---|---|
| Instrument | Bruker D8 Advance |
| Detector | LYNXEYE (1D mode), open angle: 2.948°, |
| | scan mode: continuous scan |
| Radiation | CuKα (0.15418 nm) |
| Monochromator | Nickel filter |
| X-ray generator power | 40 kV, 40 mA |
| Goniometer radius | 280 mm |
| Step size | 0.0164° (2-theta value) |
| Time per step | 0.3 second per step |
| Scan range | 2° to 40° (2-theta value) |
| Scan time | About 768 seconds |
| Slits | Primary: fixed illuminated sample |
| | size 10 mm; secondary: open |
| | angle 2.2°, axial soller: 2.5° |

### Instrumentation

Microwave: All microwave reactions were conducted in a Biotage Initiator, irradiating at 0-400 W from a magnetron at 2.45 GHz with Robot Eight/Robot Sixty processing capacity, unless otherwise stated.
UPLC-MS and MS analytical Methods: Using Waters Acquity UPLC with Waters SQ detector.
UPLC-MS-1: Acquity HSS T3; particle size: 1.8 μm; column size: 2.1×50 mm; eluent A: H₂O+0.05% HCOOH+3.75 mM ammonium acetate; eluent B: CH₃CN+0.04% HCOOH; gradient: 5 to 98% B in 1.40 min then 98% B for 0.40 min; flow rate: 1 mL/min; column temperature: 60° C.
UPLC-MS-3: Acquity BEH C18; particle size: 1.7 μm; column size: 2.1×50 mm; eluent A: H₂O+4.76% isopropanol+0.05% HCOOH+3.75 mM ammonium acetate; eluent B: isopropanol+0.05% HCOOH; gradient: 1 to 98% B in 1.7 min then 98% B for 0.1 min min; flow rate: 0.6 mL/min; column temperature: 80° C.
UPLC-MS-4: Acquity BEH C18; particle size: 1.7 μm; column size: 2.1×100 mm; eluent A: H₂O+4.76% isopropanol+0.05% HCOOH+3.75 mM ammonium acetate; eluent B: isopropanol+0.05% HCOOH; gradient: 1 to 60% B in 8.4 min then 60 to 98% B in 1 min; flow rate: 0.4 mL/min; column temperature: 80° C.
UPLC-MS-6: Acquity BEH C18; particle size: 1.7 μm; column size: 2.1×50 mm; eluent A: H₂O+0.05% HCOOH+3.75 mM ammonium acetate; eluent B: isopropanol+0.05% HCOOH; gradient: 5 to 98% B in 1.7 min then 98% B for 0.1 min; flow rate: 0.6 mL/min; column temperature: 80° C.

### Preparative Methods:

### Chiral SFC Methods:

C-SFC-1: column: Amylose-C NEO 5 μm; 250×30 mm; mobile phase; flow rate: 80 mL/min; column temperature: 40° C.; back pressure: 120 bar.
C-SFC-3: column: Chiralpak AD-H 5 μm; 100×4.6 mm; mobile phase; flow rate: 3 mL/min; column temperature: 40° C.; back pressure: 1800 psi.

### Abbreviations

| Abbreviation | Description |
|---|---|
| AcCN, ACN | acetonitrile |
| Ac₂O | acetic anhydride |
| AcOH | acetic acid |
| AIBN | 2,2′-azobis(2-methylpropionitrile) |
| aq. | aqueous |
| Ar | argon |
| B₂Pin₂ | 4,4,4′,4′,5,5,5′,5′-Octamethyl-2,2′-bi(1,3,2-dioxaborolane) |
| BPR | back pressure |
| brine | saturated aqueous sodium chloride |
| n-BuLi | n-butyl lithium |
| conc. | concentrated |
| DAST | N,N-diethyl-1,1,1-trifluoro-λ⁴-sulfanamine |
| DCE | dichloroethane |
| DCM | dichloromethane |
| DEA | diethylamine |
| DHP | 3,4-dihydropyran |
| DIPEA | N,N-diisopropylethylamine, N-ethyl-N-isopropylpropan-2-amine |
| DMA | N,N-dimethylacetamide |
| DMAP | N,N-dimethylpyridin-4-amine |
| DMF | N,N-Dimethylformamide |
| DMSO | dimethylsulfoxide |
| DMSO-d₆ | hexadeuterodimethyl sulfoxide |
| dppf | 1,1′-bis(diphenylphosphanyl) ferrocene |
| ee | enantiomeric excess |
| ESI | electrospray ionization |
| ESI-MS | electrospray ionization mass spectroscopy |
| EtOAc | ethyl acetate |
| GBq | gigabecquerel |
| h | Hour (s) |
| HPLC | high-performance liquid chromatography |
| IPA | 2-propanol |
| KOAc | potassium acetate |
| L/mL/μL | litre/millilitre/microlitre |
| LC-MS or LCMS | liquid chromatography and mass spectroscopy |
| M | molar |
| MeOH | methanol |
| min | minutes |
| MTBE | metbyl tert-butyl ether |
| MS | mass spectroscopy |
| MW, mw | microwave |
| m/z | mass to charge ratio |
| N | normality |
| N₂ | nitrogen |
| NaOtBu | Sodium tert-butoxide |
| NBS | N-bromosuccinimide |
| NCS | N-chlorosuccinimide |
| NIS | N-iodosuccinimide |
| NEt₃, Et₃N, TEA | triethylamine |
| PDA | Photodiode array detector |
| NMR | nuclear magnetic resonance |
| Pd(PPh₃)₄ | tetrakis(triphenylphosphane)palladium(0) |
| iPrMgCl | Isopropylmagnesium chloride |
| PTSA | p-toluenesulfonic acid |
| RM | reaction mixture |
| RP | reversed phase |
| Rt | retention time |
| RT | room temperature |
| RuPhos | 2-dicyclohexylphosphino-2′,6′-diisopropoxybiphenyl |
| RuPhos-Pd-G3 | (2-dicyclohexylphosphino-2′,6′-diisopropoxy-1,1′-biphenyl)2-(2′- |
| | amino-1,1′-biphenyl)]palladium(II) methanesulfonate |
| Sat. | saturated |
| SFC | supercritical fluid chromatography |
| SQ | Single-quadrupole |
| TBAF | Tetrabutylammonium fluoride |
| tBME, TBME, | tert-butyl methyl ether |
| TBMe | |
| TBq | terabecquerel |
| t-BuOH | tert-butanol |
| tBuXPhos-Pd-G3 | tBuXPhos-Pd-G3, [(2-Di-tert-butylphosphino-2′,4′,6′-triisopropyl- |
| | 1,1′-biphenyl)-2-(2′-amino-1,1′-biphenyl)] palladium(II) |
| | methanesulfonate |
| TFA | trifluoroacetic acid |
| THF | tetrahydrofuran |
| TLC | thin-layer chromatography |
| T₃P | propylphosphonic anhydride |
| TsCl | tosyl chloride, 4-Methylbenzene-1-sulfonyl chloride |
| UPLC | ultra-performance liquid chromatography |
| XPhos | 2-dicyclohexylphosphino-2′,4′,6′-triisopropylbiphenyl |
| XPhos-Pd-G3 | (2-dicyclohexylphosphino-2′,4′,6′-triisopropyl-1,1′-biphenyl)[2-(2′- |
| | amino-1,1′-biphenyl)]palladium(II) methanesulfonate |

All starting materials, building blocks, reagents, acids, bases, dehydrating agents, solvents, and catalysts utilized to prepare the compounds of the present invention are either commercially available or can be produced by organic synthesis methods known to one of ordinary skill in the art. Furthermore, the compounds of the present invention can be produced by organic synthesis methods known to one of ordinary skill in the art as shown in the following examples.

The structures of all final products, intermediates and starting materials are confirmed by standard analytical spectroscopic characteristics, e.g., MS, IR, NMR. The absolute stereochemistry of representative examples of the preferred (most active) atropisomers has been determined by analyses of X-ray crystal structures of complexes in which the respective compounds are bound to the KRAS G12C mutated. In all other cases where X-ray structures are not available, the stereochemistry has been assigned by analogy, assuming that, for each pair, the atropoisomer exhibiting the highest activity in the covalent competition assay has the same configuration as observed by X-ray crystallography for the representative examples mentioned above. The absolute stereochemistry is assigned according to the Cahn Ingold Prelog rule.

### Synthesis of Intermediate C1: tert-butyl 6-(3-bromo-4-(5-chloro-6-methyl-1-(tetrahydro-2H-pyran-2-yl)-1H-indazol-4-yl)-5-methyl-H-pyrazol-1-yl)-2-azaspiro[3.3]heptane-2-carboxylate

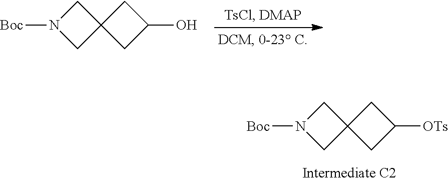 [SMILES: CC(C)(C)OC(=O)N1CC2(CC(O)C2)C1 ; Cc1ccc(S(=O)(=O)OC2CC3(C2)CN(C(=O)OC(C)(C)C)C3)cc1]

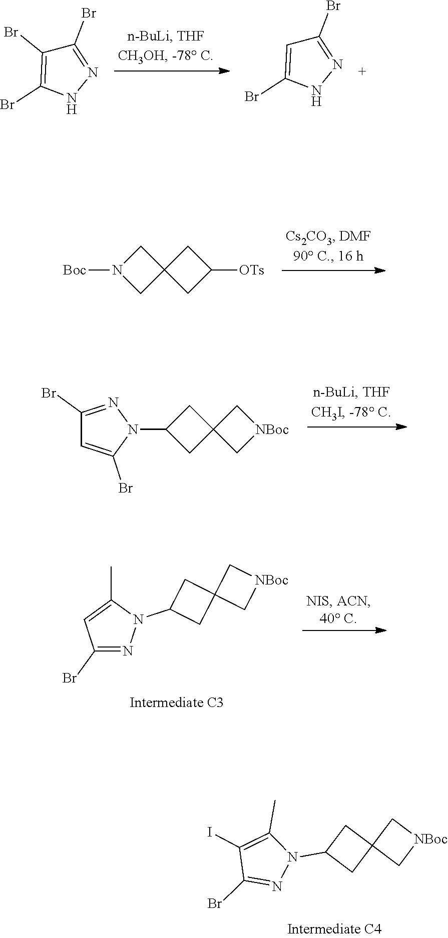 [SMILES: Brc1n[nH]c(Br)c1Br ; Brc1cc(Br)[nH]n1 ; Cc1ccc(S(=O)(=O)OC2CC3(C2)CN(C(=O)OC(C)(C)C)C3)cc1 ; CC(C)(C)OC(=O)N1CC2(CC(n3nc(Br)cc3Br)C2)C1 ; Cc1cc(Br)nn1C1CC2(C1)CN(C(=O)OC(C)(C)C)C2 ; Cc1c(I)c(Br)nn1C1CC2(C1)CN(C(=O)OC(C)(C)C)C2]

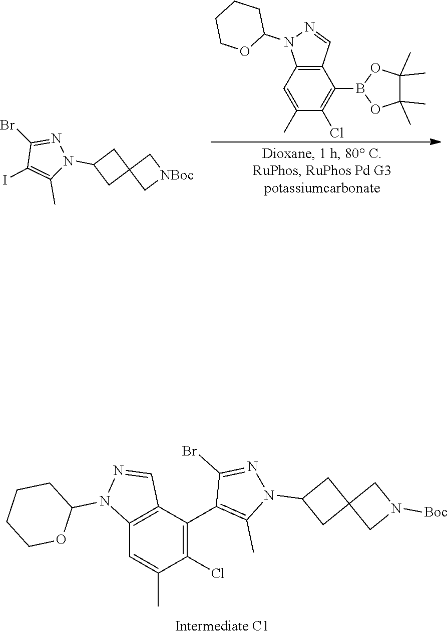 [SMILES: Cc1c(I)c(Br)nn1C1CC2(C1)CN(C(=O)OC(C)(C)C)C2 ; Cc1cc2c(cnn2C2CCCCO2)c(B2OC(C)(C)C(C)(C)O2)c1Cl ; Cc1cc2c(cnn2C2CCCCO2)c(-c2c(Br)nn(C3CC4(C3)CN(C(=O)OC(C)(C)C)C4)c2C)c1Cl]

### Step C.1: tert-butyl 6-(tosyloxy)-2-azaspiro[3.3]heptane-2-carboxylate (Intermediate C2)

To a solution of tert-butyl 6-hydroxy-2-azaspiro[3.3]heptane-2-carboxylate [1147557-97-8](2.92 kg, 12.94 mmol) in DCM (16.5 L) were added DMAP (316.12 g, 2.59 mol) and TsCl (2.96 kg, 15.52 mol) at 20° C.-25° C. To the reaction mixture was added dropwise Et₃N (2.62 kg, 25.88 mol) at 10° C.-20° C. The reaction mixture was stirred 0.5 h at 5° C.-15° C. and then was stirred 1.5 h at 18° C.-28° C. After completion of the reaction, the reaction mixture was concentrated under vacuum. To the residue was added NaCl (5% in water, 23 L) followed by extraction with EtOAc (23 L). The combined aqueous layers were extracted with EtOAc (10 L×2). The combined organic layers were washed with NaHCO₃ (3% in water, 10 L×2)) and concentrated under vacuum to give the title compound. ¹H NMR (400 MHz, DMSO-d₆) δ 7.81-7.70 (m, 2H), 7.53-7.36 (m, 2H), 4.79-4.62 (m, 1H), 3.84-3.68 (m, 4H), 2.46-2.38 (m, 5H), 2.26-2.16 (m, 2H), 1.33 (s, 9H). UPLC-MS-1: Rt=1.18 min; MS m/z [M+H]⁺; 368.2.

### Step C.2: 3,5-dibromo-1H-pyrazole

To a solution of 3,4,5-tribromo-1H-pyrazole [17635-44-8] (55.0 g, 182.2 mmol) in anhydrous THF (550 mL) was added at −78° C. n-BuLi (145.8 mL, 364.5 mmol) dropwise over 20 min maintaining the internal temperature at −78° C./−60° C. The RM was stirred at this temperature for 45 min. Then the reaction mixture was carefully quenched with MeOH (109 mL) at −78° C. and stirred at this temperature for 30 min. The mixture was allowed to reach to 0° C. and stirred for 1 h. Then, the mixture was diluted with EtOAc (750 mL) and HCl (0.5 N, 300 mL) was added. The layers were concentrated under vacuum. The crude residue was dissolved in DCM (100 mL), cooled to −50° C. and petroleum ether (400 mL) was added. The precipitated solid was filtered and washed with n-hexane (250 mL×2) and dried under vacuum to give the title compound. ¹H NMR (400 MHz, DMSO-d₆) δ 13.5 (br s, 1H), 6.58 (s, 1H).

### Step C.3: tert-butyl 6-(3,5-dibromo-1H-pyrazol-1-yl)-2-azaspiro[3.3]heptane-2-carboxylate

To a solution of tert-butyl 6-(tosyloxy)-2-azaspiro[3.3]heptane-2-carboxylate (Intermediate C2) (Step C.1, 900 g, 2.40 mol) in DMF (10.8 L) was added Cs₂CO₃ (1988 g, 6.10 mol) and 3,5-dibromo-1H-pyrazole (Step C.2, 606 g, 2.68 mol) at 15° C. The reaction mixture was stirred at 90° C. for 16 h. The reaction mixture was poured into ice-water/brine (80 L) and extracted with EtOAc (20 L). The aqueous layer was re-extracted with EtOAc (10 L×2). The combined organic layers were washed with brine (10 L), dried (Na₂SO₄), filtered, and concentrated under vacuum. The residue was triturated with dioxane (1.8 L) and dissolved at 60° C. To the light-yellow solution was slowly added water (2.2 L), and recrystallization started after addition of 900 mL of water. The resulting suspension was cooled down to 0° C. filtered, and washed with cold water. The filtered cake was triturated with n-heptane, filtered, then dried under vacuum at 40° C. to give the title compound. ¹H NMR (400 MHz, DMSO-d₆) δ 6.66 (s, 1H), 4.86-4.82 (m, 1H), 3.96-3.85 (m, 4H), 2.69-2.62 (m, 4H), 1.37 (s, 9H); UPLC-MS-3: Rt=1.19 min; MS m/z [M+H]⁺; 420.0/422.0/424.0.

### Step C.4: tert-butyl 6-(3-bromo-5-methyl-1H-pyrazol-1-yl-2-azaspiro[3.3]heptane-2-carboxylate (Intermediate C3)

To a solution of tert-butyl 6-(3,5-dibromo-1H-pyrazol-1-yl)-2-azaspiro[3.3]heptane-2-carboxylate (Step C.3, 960 g, 2.3 mol) in THF (9.6 L) was added n-BuLi (1.2 L, 2.5 mol) dropwise at −80° C. under an inert atmosphere. The reaction mixture was stirred 10 min at −80° C. To the reaction mixture was then added dropwise iodomethane (1633 g, 11.5 mol) at −80° C. After stirring for 5 min at −80° C., the reaction mixture was allowed to warm up to 18° C. The reaction mixture was poured into sat. aq. NH₄Cl solution (4 L) and extracted with DCM (10 L). The separated aqueous layer was re-extracted with DCM (5 L) and the combined organic layers were concentrated under vacuum. The crude product was dissolved in 1,4-dioxane (4.8 L) at 60° C., then water (8.00 L) was added dropwise slowly. The resulting suspension was cooled to 17° C. and stirred for 30 min. The solid was filtered, washed with water, and dried under vacuum to give the title compound. ¹H NMR (400 MHz, DMSO-d₆) δ 6.14 (s, 1H), 4.74-4.66 (m, 1H), 3.95-3.84 (m, 4H), 2.61-2.58 (m, 4H), 2.20 (s, 3H), 1.37 (s, 9H); UPLC-MS-1: Rt=1.18 min; MS m/z [M+H]⁺; 356.1/358.1.

### Step C.5: tert-butyl 6-(3-bromo-4-iodo-5-methyl-1H-pyrazol-1-yl)-2-azaspiro[3.3]heptane-2-carboxylate (Intermediate C4)

To a solution of tert-butyl 6-(3-bromo-5-methyl-1H-pyrazol-1-yl)-2-azaspiro[3.3]heptane-2-carboxylate (Intermediate C3) (Step C.4, 350 g, 0.980 mol) in acetonitrile (3.5 L) was added NIS (332 g, 1.47 mol) at 15° C. The reaction mixture was stirred at 40° C. for 6 h. After completion of the reaction, the reaction mixture was diluted with EtOAc (3 L) and washed with water (5 L×2). The organic layer was washed with Na₂SO₃ (10% in water, 2 L), with brine (2 L), was dried (Na₂SO₄), filtered, and concentrated under vacuum to give the title compound. ¹H NMR (400 MHz, DMSO-d₆) δ 4.81-4.77 (m, 1H), 3.94-3.83 (m, 4H), 2.61-5.59 (m, 4H), 2.26 (s, 3H), 1.37 (s, 9H); UPLC-MS-1: Rt=1.31 min; MS m/z [M+H]⁺; 482.0/484.0.

### Step C.6: tert-butyl 6-(3-bromo-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Intermediate C1)

To a stirred suspension of tert-butyl 6-(3-bromo-4-iodo-5-methyl-1H-pyrazol-1-yl)-2-azaspiro[3.3]heptane-2-carboxylate (Intermediate C4) (Step C.5, 136 g, 282 mmol) and 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 116 g, 310 mmol) in 1,4-dioxane (680 mL) was added aqueous K₃PO₄ (2M, 467 mL, 934 mmol) followed by RuPhos (13.1 g, 28.2 mmol) and RuPhos-Pd-G3 (14.1 g, 16.9 mmol). The reaction mixture was stirred at 80° C. for 1 h under inert atmosphere. After completion of the reaction, the reaction mixture was poured into 1M aqueous NaHCO₃ solution (1 L) and extracted with EtOAc (1 L×3). The combined organic layers were washed with brine (1 L×3), dried (Na₂SO₄), filtered, and concentrated under vacuum. The crude residue was purified by normal phase chromatography (eluent: Petroleum ether/EtOAc from I/O to 0/l) to give a yellow oil. The oil was dissolved in petroleum ether (1 L) and MTBE (500 mL), then concentrated in vacuo to give the title compound. ¹H NMR (400 MHz, DMSO-d₆) δ 7.81 (s, 1H), 7.66 (s, 1H), 5.94-5.81 (m, 1H), 4.90-4.78 (m, 1H), 3.99 (br s, 2H), 3.93-3.84 (m, 3H), 3.81-3.70 (m, 1H), 2.81-2.64 (m, 4H), 2.52 (s, 3H), 2.46-2.31 (n, 1H), 2.11-1.92 (m, 5H), 1.82-1.67 (m, 1H), 1.64-1.52 (m, 2H), 1.38 (s, 9H); UPLC-MS-3: Rt=1.30 min; MS m/z [M+H]⁺; 604.1/606.1.

### Synthesis of Intermediate D1: 5-chloro-6-methyl-1-(tetrahydro-2H-pyran-2-yl)-4-(4,4,5,5-tetramethyl-1,3,2-dioxaborolan-2-yl)-1H-indazole

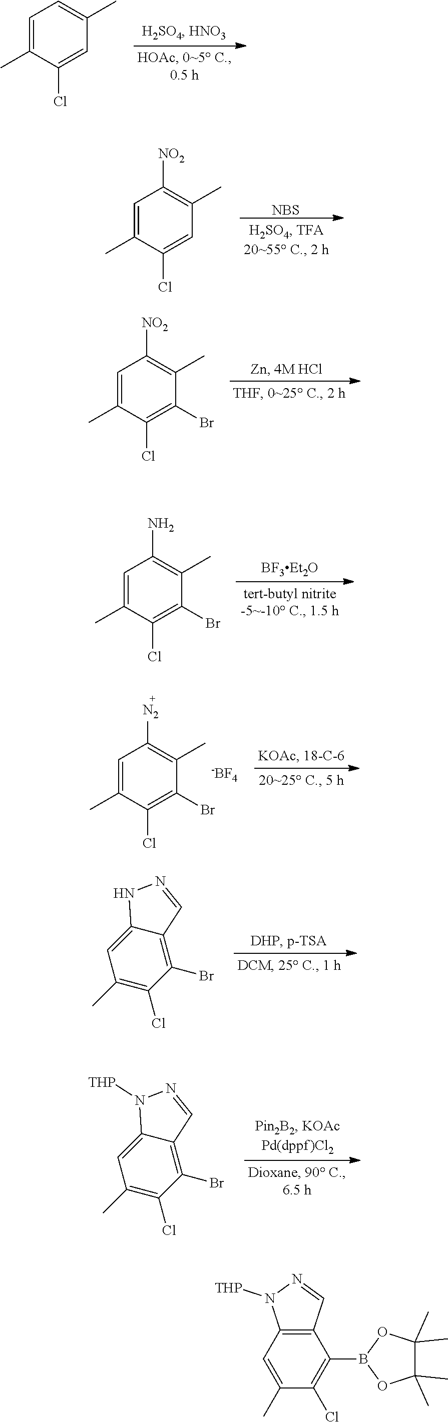

### Step D.1: 1-chloro-2,5-dimethyl-4-nitrobenzene

To an ice-cooled solution of 2-chloro-1,4-dimethylbenzene (3.40 kg, 24.2 mol) in AcOH (20.0 L) was added H₂SO₄ (4.74 kg, 48.4. mol, 2.58 L) followed by a dropwise addition (dropping funnel) of a cold solution of HNO₃ (3.41 kg, 36.3 mol, 2.44 L, 67.0% purity) in H₂SO₄ (19.0 kg, 193. mol, 10.3 L). The reaction mixture was then allowed to stir at 0-5° C. for 0.5 h. The reaction mixture was poured slowly into crushed ice (35.0 L) and the yellow solid precipitated out. The suspension was filtered and the cake was washed with water (5.00 L×5) to give a yellow solid which was suspended in MTBE (2.00 L) for 1 h, filtered, and dried to give the title compound as a yellow solid. ¹H NMR (400 MHz, CDCl₃) δ 7.90 (s, 1H), 7.34 (s, 1H), 2.57 (s, 3H), 2.42 (s, 3H).

### Step D.2: 3-bromo-2-chloro-1,4-dimethyl-5-nitrobenzene

To a cooled solution of 1-chloro-2,5-dimethyl-4-nitrobenzene (Step D.1, 2.00 kg, 10.8 mol) in TFA (10.5 L) was slowly added concentrated H₂SO₄ (4.23 kg, 43.1 mol, 2.30 L) and the reaction mixture was stirred at 20° C. NBS (1.92 kg, 10.8 mol) was added in small portions and the reaction mixture was heated at 55° C. for 2 h. The reaction mixture was cooled to 25° C., then poured into crushed ice solution to obtain a pale white precipitate which was filtered through vacuum, washed with cold water and dried under vacuum to give the title compound as a yellow solid which was used without further purification in the next step. ¹H NMR (400 MHz, CDCl₃) δ 7.65 (s, 1H), 2.60 (s, 3H), 2.49 (s, 3H).

### Step D.3: 3-bromo-4-chloro-2,5-dimethylaniline

To an ice-cooled solution of 3-bromo-2-chloro-1,4-dimethyl-5-nitrobenzene (Step D.2, 2.75 kg, 10.4 mol) in THF (27.5 L) was added HCl (4M, 15.6 L) then Zn (2.72 kg, 41.6 mol) in small portions. The reaction mixture was allowed to stir at 25° C. for 2 h. The reaction mixture was basified by addition of a sat. aq. NaHCO₃ solution (untill pH=8). The mixture was diluted with EtOAc (2.50 L) and stirred vigorously for 10 min and then filtered through a pad of celite. The organic layer was separated and the aqueous layer was re-extracted with EtOAc (3.00 L×4). The combined organic layers were washed with brine (10.0 L), dried (Na₂SO₄), filtered and concentrated under vacuum to give the title compound as a yellow solid which was used without further purification in the next step. ¹H NMR (400 MHz, DMSO-d₆) δ 6.59 (s, 1H), 5.23 (s, 2H), 2.22 (s, 3H), 2.18 (s, 3H).

### Step D.4: 3-bromo-4-chloro-2,5-dimethylbenzenediazonium tetrafluoroborate

BF₃·Et₂O (2.00 kg, 14.1 mol, 1.74 L) was dissolved in DCM (20.0 L) and cooled to −5 to −10° C. under nitrogen atmosphere. A solution of 3-bromo-4-chloro-2,5-dimethylaniline (Step D.3, 2.20 kg, 9.38 mol) in DCM (5.00 L) was added to above reaction mixture and stirred for 0.5 h. Tert-butyl nitrite (1.16 kg, 11.3 mol, 1.34 L) was added dropwise and the reaction mixture was stirred at the same temperature for 1.5 h. TLC (petroleum ether:EtOAc=5:1) showed that starting material (R_{f}=0.45) was consumed completely. MTBE (3.00 L) was added to the reaction mixture to give a yellow precipitate, which was filtered through vacuum and washed with cold MTBE (1.50 L×2) to give the title compound as a yellow solid which was used without further purification in the next step.

### Step D.5: 4-bromo-5-chloro-6-methyl-1H-indazole

To 18-Crown-6 ether (744 g, 2.82 mol) in chloroform (20.0 L) was added KOAc (1.29 kg, 13.2 mol) and the reaction mixture was cooled to 20° C. Then 3-bromo-4-chloro-2,5-dimethylbenzenediazonium tetrafluoroborate (Step D.4, 3.13 kg, 9.39 mol) was added slowly. The reaction mixture was then allowed to stir at 25° C. for 5 h. After completion of the reaction, the reaction mixture was poured into ice cold water (10.0 L), and the aqueous layer was extracted with DCM (5.00 L×3). The combined organic layers were washed with a sat. aq. NaHCO₃ solution (5.00 L), brine (5.00 L), dried (Na₂SO₄), filtered and concentrated under vacuum to give the title compound as a yellow solid. ¹H NMR (600 MHz, CDCl₃) δ 10.42 (br s, 1H), 8.04 (s, 1H), 7.35 (s, 1H), 2.58 (s, 3H). UPLC-MS-1: Rt=1.02 min; MS m/z [M+H]⁺; 243/245/247.

### Step D.6: 4-bromo-5-chloro-6-methyl-1-(tetrahydro-2H-pyran-2-yl)-1H-indazole

To a solution of PTSA (89.8 g, 521 mmol) and 4-bromo-5-chloro-6-methyl-1H-indazole (Step D.5, 1.28 kg, 5.21 mol) in DCM (12.0 L) was added DHP (658 g, 7.82 mol, 715 mL) dropwise at 25° C. The mixture was stirred at 25° C. for 1 h. After completion the reaction, the reaction mixture was diluted with water (5.00 L) and the organic layer was separated. The aqueous layer was re-extracted with DCM (2.00 L). The combined organic layers were washed with a sat. aq. NaHCO₃ solution (1.50 L), brine (1.50 L), dried over Na₂SO₄, filtered and concentrated under vacuum. The crude residue was purified by normal phase chromatography (eluent: Petroleum ether/EtOAc from 100/1 to 10/1) to give the title compound as a yellow solid. ¹H NMR (600 MHz, DMSO-d₆) δ 8.04 (s, 1H), 7.81 (s, 1H), 5.88-5.79 (m, 1H), 3.92-3.83 (m, 1H), 3.80-3.68 (m, 1H), 2.53 (s, 3H), 2.40-2.32 (m, 1H), 2.06-1.99 (m, 1H), 1.99-1.93 (m, 1H), 1.77-1.69 (m, 1H), 1.60-1.56 (m, 2H). UPLC-MS-6: Rt=1.32 min; MS m/z [M+H]⁺; 329.0/331.0/333.0

### Step D.7: 5-chloro-6-methyl-1-(tetrahydro-2H-pyran-2-yl)-4-(4,4,5,5-tetramethyl-1,3,2-dioxaborolan-2-yl)-1H-indazole (Intermediate D.1)

A suspension of 4-bromo-5-chloro-6-methyl-1-(tetrahydro-2H-pyran-2-yl)-1H-indazole (Step D.6, 450 g, 1.37 mol), KOAc (401 g, 4.10 mol) and B₂Pin₂ (520 g, 2.05 mol) in 1,4-dioxane (3.60 L) was degassed with nitrogen for 0.5 h. Pd(dppf)Cl₂·CH₂Cl₂ (55.7 g, 68.3 mmol) was added and the reaction mixture was stirred at 90° C. for 6 h. The reaction mixture was filtered through diatomite and the filter cake was washed with EtOAc (1.50 L×3). The mixture was concentrated under vacuum to give a black oil which was purified by normal phase chromatography (eluent: Petroleum ether/EtOAc from 100/1 to 10/1) to give the desired product as brown oil. The residue was suspended in petroleum ether (250 mL) for 1 h to obtain a white precipitate. The suspension was filtered, dried under vacuum to give the title compound as a white solid. ¹H NMR (400 MHz, CDCl₃) δ 8.17 (d, 1H), 7.52 (s, 1H), 5.69-5.66 (m, 1H), 3.99-3.96 (m, 1H), 3.75-3.70 (m, 1H), 2.51 (d, 4H), 2.21-2.10 (m, 1H), 2.09-1.99 (m, 1H), 1.84-1.61 (m, 3H), 1.44 (s, 12H); UPLC-MS-6: Rt=1.29 min; MS m/z [M+H]⁺; 377.1/379.

### Synthesis of Compound A

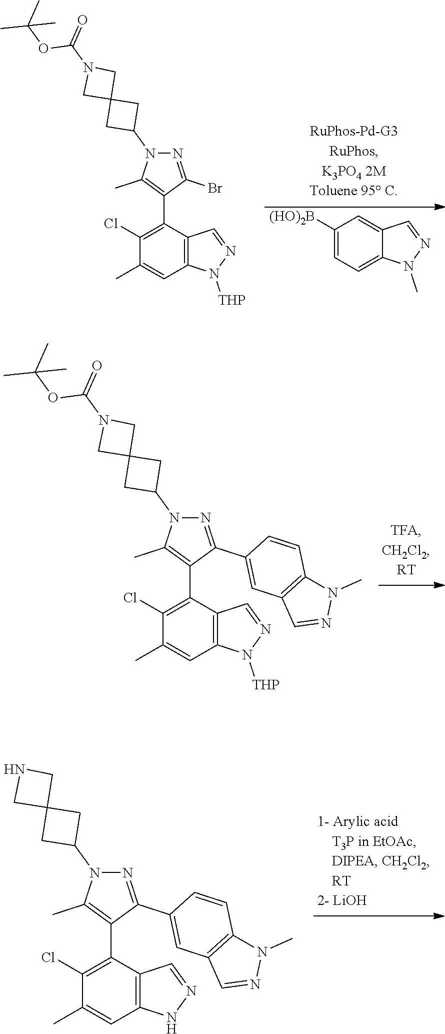

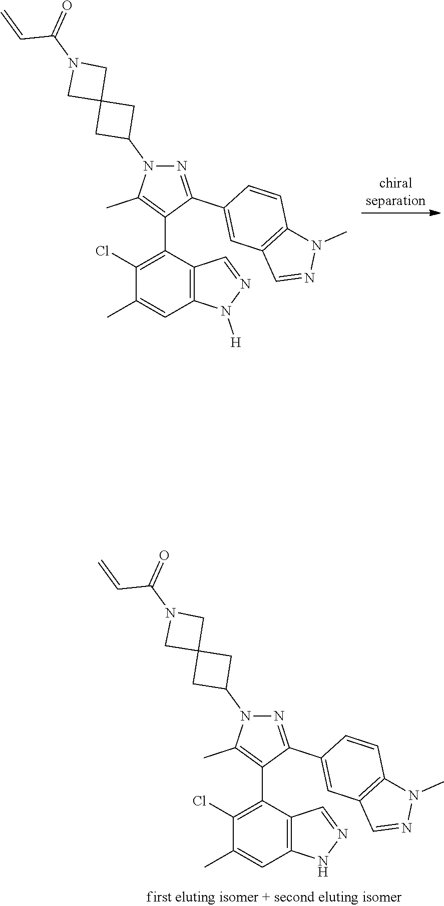

### Step 1: Tert-butyl 6-(4-(5-chloro-6-methyl-1-(tetrahydro-2H-pyran-2-yl)-1H-indazol-4-yl)-5-methyl-3-(1-methyl-1H-indazol-5-yl)-1H-pyrazol-1-yl)-2-azaspiro[3.3]heptane-2-carboxylate

In a 500 mL flask, tert-butyl 6-(3-bromo-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Intermediate C1, 10 g, 16.5 mmol), (1-methyl-1H-indazol-5-yl)boronic acid (6.12 g, 33.1 mmol), RuPhos (1.16 g, 2.48 mmol) and RuPhos-Pd-G3 (1.66 g, 1.98 mmol) were suspended in toluene (165 mL) under argon. K₃PO₄ (2M, 24.8 mL, 49.6 mmol) was added and the reaction mixture was placed in a preheated oil bath (95° C.) and stirred for 45 min. The reaction mixture was poured into a sat. aq. NH₄Cl solution and was extracted with EtOAc (×3). The combined organic layers were washed with a sat. aq. NaHCO₃ solution, dried (phase separator) and concentrated under reduced pressure. The crude residue was diluted with THF (50 mL), SiliaMetS®Thiol (15.9 mmol) was added and the mixture swirled for 1 h at 40° C. The mixture was filtered, the filtrate was concentrated and the crude residue was purified by normal phase chromatography (eluent: MeOH in CH₂Cl₂ from 0 to 2%), the purified fractions were again purified by normal phase chromatography (eluent: MeOH in CH₂Cl₂ from 0 to 2%) to give the title compound as a beige foam. UPLC-MS-3: Rt=1.23 min; MS m/z [M+H]⁺; 656.3/658.3.

### Step 2: 5-Chloro-6-methyl-4-(5-methyl-3-(1-methyl-1H-indazol-5-yl)-1-(2-azaspiro[3.3]heptan-6-yl)-1H-pyrazol-4-yl)-1H-indazole

TFA (19.4 mL, 251 mmol) was added to a solution of tert-butyl 6-(4-(5-chloro-6-methyl-1-(tetrahydro-2H-pyran-2-yl)-1H-indazol-4-yl)-5-methyl-3-(1-methyl-1H-indazol-5-yl)-1H-pyrazol-1-yl)-2-azaspiro[3.3]heptane-2-carboxylate (Step 1, 7.17 g, 10.0 mmol) in CH₂Cl₂ (33 mL). The reaction mixture was stirred at RT under nitrogen for 1.5 h. The RM was concentrated under reduced pressure to give the title compound as a trifluoroacetate salt, which was used without purification in the next step. UPLC-MS-3: Rt=0.74 min; MS m/z [M+H]⁺; 472.3/474.3.

### Step 3: 1-(6-(4-(5-Chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one

A mixture of acrylic acid (0.69 mL, 10.1 mmol), propylphosphonic anhydride (50% in EtOAc, 5.94 mL, 7.53 mmol) and DIPEA (21.6 mL, 126 mmol) in CH₂Cl₂ (80 mL) was stirred for 20 min at RT and then added (dropping funnel) to an ice-cooled solution of 5-chloro-6-methyl-4-(5-methyl-3-(1-methyl-1H-indazol-5-yl)-1-(2-azaspiro[3.3]heptan-6-yl)-1H-pyrazol-4-yl)-1H-indazole trifluoroacetate (Step 2, 6.30 mmol) in CH₂Cl₂ (40 mL). The reaction mixture was stirred at RT under nitrogen for 15 min. The RM was poured into a sat. aq. NaHCO₃ solution and extracted with CH₂Cl₂ (×3). The combined organic layers were dried (phase separator) and concentrated. The crude residue was diluted with THF (60 mL) and LiOH (2N, 15.7 mL, 31.5 mmol) was added. The mixture was stirred at RT for 30 min until disappearance (UPLC) of the side product resulting from the reaction of the acryloyl chloride with the free NH group of the indazole then was poured into a sat. aq. NaHCO₃ solution and extracted with CH₂Cl₂ (3×). The combined organic layers were dried (phase separator) and concentrated. The crude residue was purified by normal phase chromatography (eluent: MeOH in CH₂Cl₂ from 0 to 5%) to give the title compound. The isomers were separated by chiral SFC (C-SFC-1; mobile phase: CO₂/[IPA+0.1% Et₃N]: 69/31) to give Compound A, i.e. a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one, as the second eluting peak (white powder): ¹H NMR (600 MHz. DMSO-d₆) δ 13.1 (s, 1H), 7.89 (s, 1H), 7.59 (s, 1H), 7.55 (s, 1H), 7.42 (m, 2H), 7.30 (d, 1H), 6.33 (m, 1H), 6.12 (m, 1H), 5.68 (m, 1H), 4.91 (m, 1H), 4.40 (s, 1H), 4.33 (s, 1H), 4.11 (s, 1H), 4.04 (s, 1H), 3.95 (s, 3H), 2.96-2.86 (m, 2H), 2.83-2.78 (m, 2H), 2.49 (s, 3H), 2.04 (s, 3H); UPLC-MS-4: Rt=4.22 min; MS m/z [M+H]) 526.3/528.3; C-SFC-3 (mobile phase: CO₂/[IPA+0.1% Et₃N]: 67/33): Rt=2.23 min. The compound of Example 1 is also referred to as “Compound A”.

The atropisomer of Compound A, a(S)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one was obtained as the first eluting peak: C-SFC-3 (mobile phase: CO₂/[IPA+0.1% Et₃N]: 67/33): Rt=1.55 min.

### Example 2: Synthesis of Crystalline Forms of Compound A

Crystalline forms of Compound A such as the ones described below are particularly suitable in the methods and uses of the invention.

### Example 2a: Crystalline Isopropyl Alcohol (IPA) Solvate of Compound A and Crystalline Hydrate (Modification HA) Form of Compound A

25 mg of Compound A (obtained from Example 1 above) was added to 0.1 mL of 2-propanol. The resulting clear solution was stirred at 25° C. for 3 days, after which crystalline solid precipitated out. The solid was collected by centrifuge filtration and dried at ambient condition overnight. The wet cake was characterized as crystalline isopropyl (IPA) solvate of Compound A. Drying of the wet cake at ambient condition overnight provided crystalline hydrate (Modification HA) form.

Crystalline hydrate (Modification HA) form of Compound A was analysed by XRPD and the most characteristic peaks are shown in the Table below.

In particular, the most characteristic peaks of the XRPD pattern of the crystalline hydrate (Modification HA) form may be selected from one, two, three or four peaks having an angle of refraction 20 values (CuKα λ=1.5418 Å) selected from the group consisting of 8.2°, 11.60, 12.9° and 18.8°.

| Index | Two-Theta | d value | Relative intensity | Intensity |
|---|---|---|---|---|
| 1 | 8.2° | 10.72 Å | 100% | Strong |
| 2 | 11.6° | 7.60 Å | 11% | Weak |
| 3 | 12.1° | 7.30 Å | 10% | Weak |
| 4 | 12.9° | 6.87 Å | 14% | Medium |
| 5 | 14.6° | 6.05 Å | 21% | Medium |
| 6 | 16.2° | 5.47 Å | 15% | Medium |
| 2 | 18.8° | 4.73 Å | 28% | Medium |
| 8 | 20.4° | 4.34 Å | 18% | Medium |
| 9 | 24.1° | 3.69 Å | 29% | Medium |

Crystalline IPA solvate form of Compound A was analysed by XRPD and the most characteristic peaks are shown in the Table below.

In particular, the most characteristic peaks of the XRPD pattern of the crystalline IPA solvate form may be selected from one, two, or three peaks having an angle of refraction 20 values (CuKα λ=1.5418 Å) selected from the group consisting of 7.5°, 12.5° and 17.6°.

| Index | Two-Theta | d value | Relative intensity | Intensity |
|---|---|---|---|---|
| 1 | 7.5° | 11.77 | 100% | Strong |
| 2 | 12.5° | 7.08 Å | 20% | Medium |
| 3 | 15.5° | 5.73 Å | 14% | Low |
| 4 | 16.4° | 5.42 Å | 8% | Low |
| 5 | 17.6° | 5.04 Å | 28% | Medium |
| 6 | 21.4° | 4.15 Å | 11% | Low |
| 7 | 24.4° | 3.64 Å | 8% | Low |

### Example 2b: Crystalline Ethanol (EtOH) Solvate of Compound A and Crystalline Hydrate (Modification HA) Form of Compound A

25 mg of Compound A (obtained from Example 1 above) was added to 0.1 ml, of ethanol. The resulting clear solution was stirred at 25° C. for 3 days. Crystalline hydrate (Modification HA) form of Compound A obtained in example 1 was added as seeds to the resulting solution. The resulting suspension was equilibrated for another 1 day, after which a solid precipitated out. The solid was collected by centrifuge filtration and dried at ambient condition overnight. The wet cake was characterized as crystalline ethanol solvate, which after drying at ambient condition overnight, produced crystalline hydrate (Modification HA).

Alternatively, 3.1 g of Compound A was added to 20 mL of ethanol, the resulting clear solution was stirred at 25° C. for 20 mins. Approximately 50 mg crystalline hydrate (Modification HA) (obtained above) was added as seeds, and the resulting mixture was equilibrated at 25° C. for 6 hours. The resulting suspension was filtered and the wet cake was characterized as crystalline ethanol solvate. The solid was then dried at ambient condition (25° C., 60-70% Relative Humidity) for 3 days, 2.8 g of Compound A hydrate Modification HA was obtained with a yield of 90%.

Crystalline ethanol solvate form of Compound A was analysed by XRPD and the most characteristic peaks are shown in the Table below.

In particular, the most characteristic peaks of the XRPD pattern of the crystalline ethanol solvate form may be selected from one, two, or three or four peaks having an angle of refraction 20 values (CuKα λ=1.5418 Å) selected from the group consisting of 7.9°, 12.7°, 18.2° and 23.1°.

| Index | Two-Theta | d value | Relative intensity | Intensity |
|---|---|---|---|---|
| 1 | 7.9° | 11.20 Å | 100% | Strong |
| 2 | 12.7° | 6.99 Å | 24% | Medium |
| 3 | 13.1° | 6.76 Å | 12% | weak |
| 4 | 15.5° | 5.70 Å | 18% | weak |
| 5 | 15.9° | 5.58 Å | 11% | weak |
| 6 | 16.9° | 5.24 Å | 11% | weak |
| 7 | 18.2° | 4.88 Å | 32% | medium |
| 8 | 18.6° | 4.77 Å | 17% | weak |
| 9 | 23.1° | 3.85 Å | 26% | Medium |

### Example 2c: Alternative Preparation of Crystalline Hydrate (Modification HA) Preparation

25 mg of Compound A (obtained from Example 1 above) was added to 0.1 mL of methanol. The resulting clear solution was stirred at 25° C. for 3 days. Crystalline hydrate (Modification HA) obtained in example 1 was added as seeds to the resulting solution. The resulting suspension was equilibrated for another 1 day, after which a solid precipitated out. The solid was collected by centrifuge filtration and dried at ambient condition overnight. After drying at ambient condition overnight, the wet cake produced crystalline hydrate (Modification HA).

### Example 2d: Crystalline Propylene Glycol Solvate Preparation and Hydrate (Modification HA) Preparation

25 mg of Compound A (obtained from Example 1 above) was added to 0.1 mL of propylene glycol. The resulting suspension was stirred at 50° C. for 1 week. The solid was collected by centrifuge filtration. The wet cake obtained after filtration was characterized as crystalline propylene glycol solvate. After drying of the cake at ambient condition for 1 week, crystalline hydrate (Modification HA) was obtained.

Crystalline propylene glycol solvate form of Compound A was analysed by XRPD and the most characteristic peaks are shown in the Table below.

In particular, the most characteristic peaks of the XRPD pattern of the crystalline propylene glycol solvate form may be selected from one, two, or three or four peaks having an angle of refraction 20 values (CuKα λ=1.5418 Å) selected from the group consisting of 7.3°, 13.2°, 18.0° and 25.5°.

| Index | Two-Theta | d value | Relative intensity | Intensity |
|---|---|---|---|---|
| 1 | 7.3° | 12.15 Å | 34% | weak |
| 2 | 13.2° | 6.70 Å | 87% | Strong |
| 3 | 15.6° | 5.69 Å | 37% | weak |
| 4 | 16.2° | 5.48 Å | 56% | Medium |
| 5 | 18.0° | 4.92 Å | 64% | Medium |
| 6 | 22.5° | 3.96 Å | 100% | Strong |
| 7 | 22.8° | 3.90 Å | 35% | weak |
| 8 | 23.2° | 3.83 Å | 33% | weak |
| 9 | 25.1° | 3.55 Å | 37% | weak |

### Example 3: Compound A Binds Under the Switch II Loop of KRAS G12C, Exploiting Unique Interactions with KRAS G12C and Inhibits KRASG12C Cellular Signaling and Proliferation Potently and Selectively

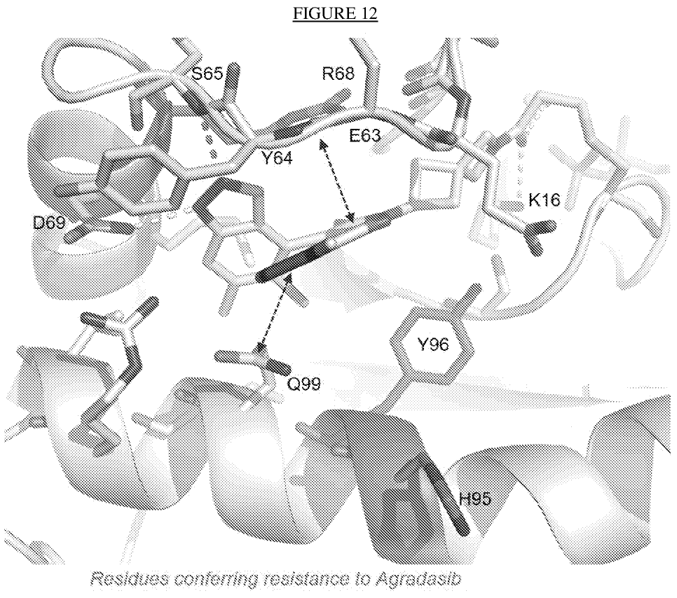

Compound A binds under the switch II loop of KRAS G12C, exploiting unique interactions with KRAS G12C compared to other KRAS G12C inhibitors such as sotorasib and adagrasib. For example, the methylindazolyl moiety of Compound A forms stacking interactions with the Tyr64 and Glu63 backbone and interacts with the side chain of Gln99. As a result, this methylindazolyl moiety is sandwiched between the switch II and the Gln99 backbone, stabilizing this switch II conformation. The 44 spiro linker growing towards the C12 moiety is attached onto the pyrazole ring; this results in a different way to occupy the binding site and to optimally position the acrylamide to interact with the Lys16 and C12 moiety. In addition, the switch II conformation is different to the switch II conformation disclosed for the published binding modes of other KRAS G12C inhibitors, e.g. sotorasib and adagrasib. (FIG. 12) Compound A may therefore be useful for the treatment of patients suffering from a cancer, specially a cancer as described herein, which has become resistant or refractory to treatment with another KRAS G12C inhibitor, e.g. sotorastib or adagrasib.

In cellular assays, Compound A demonstrated potent and selective target occupancy. Compound A selectively inhibited downstream effector protein recruitment to KRAS G12C, but not to any other RAS wild-type isoform. Compound A inhibited KRAS-driven oncogenic signaling and proliferation specifically in KRAS G12C-mutated cell lines, but not KRAS WT or MEK Q56P mutated cell lines.

TABLE
Compound A inhibits KRASG12C cellular signaling
and proliferation potently and selectively

| Biochemical assays | Value |
|---|---|
| Covalent competition (SWII pocket) k_{react}/k_{B} | 140 mM⁻¹s⁻¹ |
| KRAS^{G12C} GDP | |
| Cellular assays | Value [μM] |
| Target occupancy 6 h EC₅₀ KRAS^{G12C} | 0.03 |
| cRAF recruitment 6 h EC₅₀ | 0.02/>10/>6/>10 |
| KRAS^{G12C} vs KRAS/NRAS/HRAS WT | |
| pERK 6 h EC₅₀ KRAS^{G12C} | 0.02/>10 |
| vs MEK^{Q56P} | |
| Proliferation GI₅₀ KRAS^{G12C} vs MEK^{Q56P} | 0.03/4 |

In preclinical studies, Compound A showed deep and sustained target occupancy resulting in high efficacy in KRAS G12C mutant xenograft models. In KRAS G12C mutated xenograft models in mice, pharmacodynamic (PD) responses correlated with Compound A exposure in blood. Upon Compound A treatment, free tumor KRAS G12C levels were robustly reduced in a dose-dependent manner and correlated with inhibition of tumor expression of the MAPK pathway target gene, DUSP-6.

Moreover, daily oral Compound A treatment resulted in dose-dependent anti-tumor activity on KRASG12C xenograft models (MIA PaCa (KRASG12C pancreas) and NCI-H2122 (KRASG12C lung) xenograft models in mice. In MIA PaCa-2, Compound A produced tumor stasis at 3 mg/kg and tumor regression at 10 mg/kg, 30 mg/kg and 100 mg/kg. In NCI-H2122, Compound A produced weak tumor-growth inhibition at 10 mg/kg, a moderate tumor-growth inhibition at 30 mg/kg and approximate tumor stasis at 100 mg/kg. Likewise, a twice daily oral treatment with Compound A at 50 mg/kg also achieved approximate tumor stasis in NCI-H2122, indicating AUC as the driver for efficacy.

Overall, Compound A was well tolerated in 4-week rat and dog toxicity studies.

### Example 4: QD or BID Schedules of Compound A at the Same Daily Dose Show Same Antitumor Activity in KRAS G12C-Mutated Xenografts

The anti-tumor activity of Compound A was investigated as single-agent in a panel of KRAS G12C-mutated xenograft models across different indications: MIA PaCa-2 (PDAC); NCI-H2122, LU99, HCC-44, NCI-H2030 (NSCLC); KYSE410 (esophageal). Based on the PK profile and PK/PD relationship, doses of Compound A of 10 mg/kg, 30 mg/kg and 100 mg/kg in an oral daily schedule were chosen. Compound A inhibited the growth of all xenograft models in a dose dependent manner. Hereby, a difference in the dynamic range of the dose-response and a pattern of maximal response ranging from regression (MIA-PaCa-2, LU99) over stasis-like (HCC44, NCI-H2122) to moderate tumor growth inhibition (NCI-H2030. KYSE410) was observed across the different xenograft models, reflecting the differences of sensitivity of each model for KRASG12C inhibition.

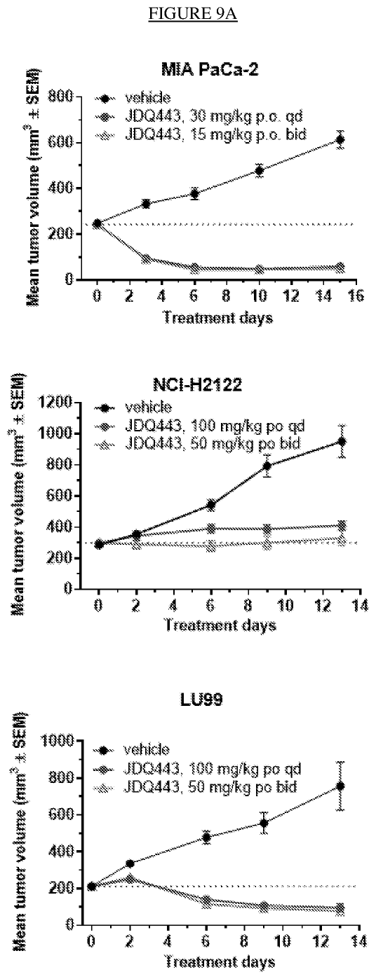

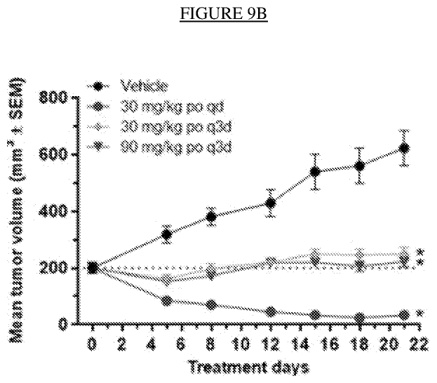

Daily oral treatment with Compound A at 30 mg/kg (qd) induced the same tumor regression in MIA PaCa-2 xenografts as the twice daily treatment at 15 mg/kg (FIG. 9). Similarly, twice daily treatment at 5 mg/kg achieved comparable regression to a daily oral treatment at 10 mg/kg. This result was also obtained in NCI-H2122 and Lu99 xenografts (“H2122” and “LU99” in FIG. 9). The 30 mg/kg dose given in a daily schedule was found to result in better tumor growth inhibition when compared to the same dose given every three days (q3d) or the same total daily dose of 90 mg/kg q3d (FIG. 9B). The efficacy correlated well with total drug concentration in blood. These data suggest an AUC-driven PD/efficacy relationship.

Daily oral administration of Compound A elicited very similar anti-tumor activity in the MIA PaCa-2 and NCI-H2122 xenografts as compared to sotorasib and to adagrasib given at the estimated clinically relevant doses. In MIA PaCa-2, tumor regression was achieved with both Compound A and sotorasib given at 10 mg/kg and 30 mg/kg with no statistically significant difference between the treatment groups. In the less sensitive model NCI-H2122, daily dosing with 100 mg/kg of Compound A, sotorasib and adagrasib induced tumor stasis, 30 mg/kg of Compound A and sotorasib led to slightly less than tumor stasis and 30 mg/kg of adagrasib had no effect on tumor growth as compared to the vehicle group

### Example 5: In Vitro Combinations of Compound A with TNO155 in KRAS G12C-Mutated NSCLC Cell Lines

10 mM stock solutions of the test compounds were dissolved in 100% DMSO, and stored them in small aliquots at −20° C.

Ten KRAS G12C-mutated non-small-cell lung carcinoma (NSCLC) cell lines: NCI-H2030, NCI-H358, NCI-H1792, HCC-44, NCI-H1373, Calu-1, NCI-H23, Lu99, NCI-H2122 and HCC-1171 were from commercially available sources and cultured at 37° C. 5% C02 in the media conditions recommended by the provider.

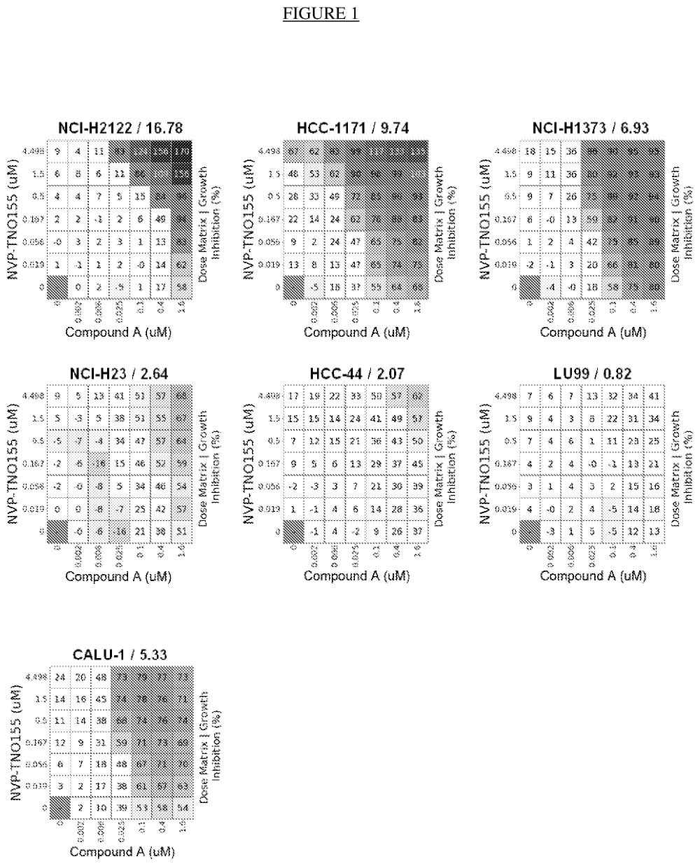

The indicated KRAS G12C-mutated human NSCLC cell lines were dispensed into 384-well tissue culture plates. Cells were treated in triplicates with DMSO, a dose range of Compound A (X axis; 2 nM up to 1.6 μM), a dose range of TNO155 (Y axis; 19 nM up to 4.5 μM) and the pairwise combination of the two agents for 7 consecutive days. After seventy-two hours, the medium was refreshed by supplementing the same volume per well of culture medium containing the corresponding compounds or DMSO. After the seven days treatment period, cell growth was determined using CellTiter-Glo® (Promega). One plate was counted prior treatment (=Day 1). The matrices in FIG. 1 report the percentage Growth Inhibition (GI) for each treatment as compared to DMSO-treated cells, with darker colors denoting greater cell growth inhibition and/or cell kill. The data was processed using classical synergy models (Loewe, Bliss). The synergy scores for the Compound A/TNO155 combinations for the following cell lines were as follows. NCI-H2122: 16.7; HCC-1171: 9.7; NCI-H1373: 6.9. Synergy scores above 2 indicate a synergistic effect.

Synergistic cell killing occurred in NCI-H2122 and HCC-1171, while synergistic cell growth inhibition was achieved in NCI-1373 and CALU-1 (see FIG. 1). Lower synergy scores were obtained in NCI-H23 and HCC-44 cell lines. The Lu99 cell line (LU99 in FIG. 1) showed no synergy in this experiment, likely due to an in-vitro effect as the combination of Compound A and TNO155 showed a clear beneficial effect in-vivo Lu99 models.

### Example 6: Anti-Tumor Efficacy of Compound A Alone and in Combination with TNO155 in Lu99 KRAS G12C Lung Carcinoma Mouse Xenograft Models

A heterozygous KRAS G12C lung cancer xenograft model, named Lu99, was used in an efficacy study in mice to study the efficacy and tolerability of Compound A, TNO155 used as single agents, and in combination.

The Lu99 human cell line originates from a Lung giant cell carcinoma of a 63-year-old male patient [Yamada et al. 1985]. It carries the allele NM_033360.4(KRAS):c.34G>T and consequently a heterozygous KRAS Gly12Cys mutation. Lu99 cells were grown in sterile conditions in a 37° C. incubator with 5% CO2 for two weeks. The cells were kept in RPMI media supplemented with 10% FCS, 2 mM L-Glutamin, 1 mM sodium pyruvate and 10 mM HEPES, and split 1:6 every 3 days. Cells were tested negative for mycoplasma and murine viruses in 2012 (Radil case number: 8270-2012). On the day of injection, cells were harvested after 8 passages in total, including passages from the vendor. Cells were resuspended in 50% HBSS and 50% Matrigel at a final concentration of 10·10⁶ cells/ml.

Experiments were performed with female nude mice (Charles River Laboratories, Crl:NU(NCr)-Foxn1nu Homozygous). The animals were housed in a 12 h light/dark cycle facility and had access toFigure sterilized food and water ad libitum. Animals were allowed to accommodate at least for 7 days.

Mice were injected subcutaneously with Lu99 human NSCLC cells to induce xenograft tumors and randomized into treatment groups when the mean tumor volume reached ˜250 mm³. Mice were then treated orally at with vehicle, Compound A at 100 mg/kg once daily, TNO155 at 10 mg/kg twice daily, or a combination of Compound A at 100 mg/kg once daily and TNO155 at 10 mg/kg twice daily.

Compound A and TNO155 were each formulated as a suspension in 0.1% Tween 80 (Fisher Scientific AG #BP338-500) and 0.5% Methylcellulose in water. The control group received a solution of 0.1% Tween 80 (Fisher Scientific AG #BP338-500) and 0.5% Methylcellulose in water.

The treatment period was between 9 to 28 days, depending on the groups. Animals treated with vehicle were terminated at day 9 and TNO155 treated animals at day 14 as their tumor volume (TV) reached the authorized limit. Animals treated with Compound A or the combination of Compound A and TNO155 were treated for 28 days, and then kept for 5 more days without any treatment.

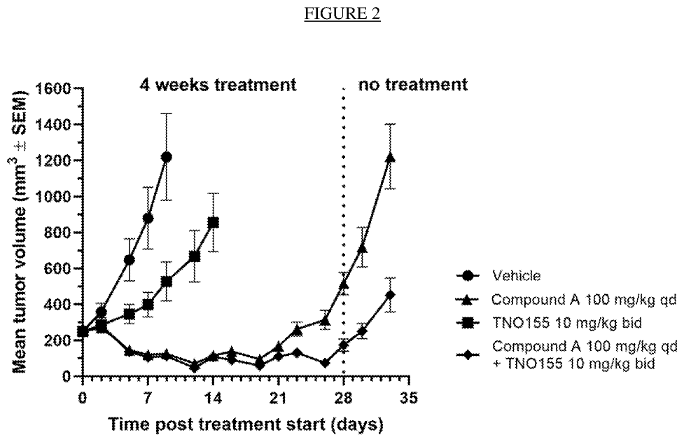

Tumor growth was monitored regularly post cell inoculation and animals were randomized into treatment groups (n=6) when TV reached appropriate volume. During the treatment period, xenograft tumor sizes were measured about 2-3 times per week manually with calipers, and the TV was estimated in mm³ using the formula: Length×Width²/2. Results are shown in FIG. 2.

While a moderate anti-tumor response with single agent TNO155 compared with vehicle group, Compound A treatment led to Lu99 tumor regression for three weeks while treatment was still ongoing. The combination of these two agents significantly improved the sustainability of response and time to relapse seen with Compound A as a single agent, leading to a tumor response similar to Compound A alone, during the three first weeks of treatment. Tumors did not regrow under treatment, unlike what was observed with treatment with Compound A alone. Nonetheless, the lung carcinoma growth resumed when treatment was ended. All animals in the study gained body weight throughout the treatment period. All treatment regimens were acceptable, and the blood exposures of both compounds were in similar range when administrated alone or in combination.

### Example 7: Anti-Tumor Efficacy of Compound A Alone and in Combination with Different Schedules of TNO155 in Lu99 KRAS G12C Lung Carcinoma Mouse Xenograft Models

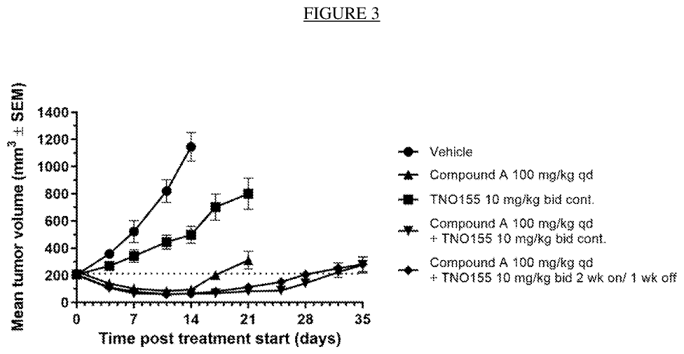

An in vivo combination study of Compound A with different schedules of TNO155 was conducted in the KRAS G12C-mutated Lu99 xenograft model in female nude mice. Mice were injected subcutaneously with Lu99 human NSCLC cells to induce xenograft tumors and randomized into treatment groups when the mean tumor volume reached ˜200 mm³. Mice were treated orally with vehicle, Compound A at 100 mg/kg once daily, TNO155 at 10 mg/kg twice daily continuous, or a combination of Compound A at 100 mg/kg once daily and TNO155 at 10 mg/kg twice daily on a continuous schedule or on a two weeks on, one week off schedule. The treatment period was between 14 to 35 days, depending on the groups. Animals treated with vehicle were terminated at day 14. TNO155 and Compound A treated animals were terminated at day 21. Animals treated with the combination of Compound A and TNO155 were treated for 35 days. Tumor volumes were recorded and are represented as mean±SEM (standard error of the mean) for each group. Anti-tumor response of treatment groups vs. vehicle group was calculated at day 14 as % T/C or % regression. Daily dosing with Compound A at 100 mg/kg induced tumor regression for approximately two weeks, followed by tumor relapse while treatment was still ongoing. TNO155 given continuously at 10 mg/kg twice daily led to slight tumor growth delay compared to the vehicle group. As shown in FIG. 3, the combination of Compound A with TNO155 significantly improved the sustainability of response and time to relapse seen with Compound A as a single agent. Hereby, the combination effect was the same regardless whether TNO155 was given at a continuous schedule or a two weeks on, one week off schedule. This means that TNO155 may only need to be administered on an intermittent schedule, to maintain clinical efficacy, or that dose interruptions due to clinical adverse effects may not be detrimental to clinical response.

### Example 8: Compound A Shows Sustained Target Occupancy In Vivo and has Dose-Dependent Anti-Tumor Activity in Mice Bearing KRAS G12C Mutated Tumor Xenografts. Adding TNO155 to Compound A Achieved Similar Target Occupancy at a Third of the Amount of Compound A Single Agent

KRASG12C inhibition can be assessed by measuring free KRASG12C levels (target occupancy) and other biomarkers in the MAPK signaling pathway, such as decreased levels of phosphorylated ERK1/2 (pERK) and downregulation of DUSP6 mRNA transcript. In H358 cancer cells, the in vitro IC50s for inhibition of KRAS G12C (target occupancy) and pERK were 20 nM, respectively, and the antiproliferation GI50 was 30 nM. In vivo, orally administered Compound A (30 mg/kg QD) achieved approximately 90% KRASG12C inhibition and 75% decrease in DUSP6 mRNA transcript in MIA PaCa-2 xenografts, causing tumor regression. In the less sensitive NCI-H2122 xenografts, orally administered JDQ443 (100 mg/kg QD) achieved approximately 80% KRASG12C inhibition and 90% decrease in DUSP6 mRNA transcript, causing stasis which is the maximum response in this model upon MAPK pathway inhibition.

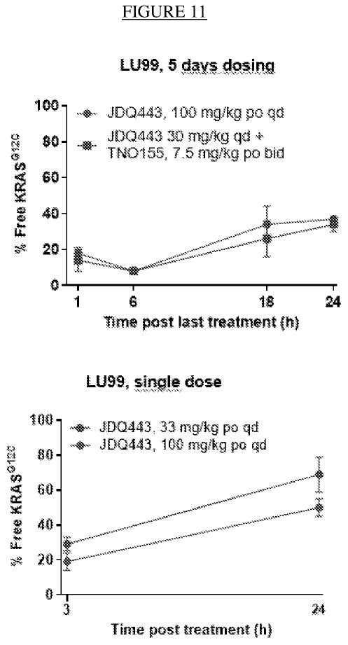

In a corresponding study, KRASG12C target occupancy was assessed after 5 days treatment of LU99 xenografts with single-agent Compound A at 100 mg/kg qd or in combination at 30 mg/kg with TNO155 at 7.5 mg/kg bid. As seen in FIG. 11 both regimens achieved comparable reduction of free KRASG12C in tumors. Hence addition of a SHP2 inhibitor can enhance the anti-tumor response of Compound A, whereby the same target occupancy is achieved at a third of the dose of the KRAS G12C inhibitor alone.

Compound A achieved an anti-tumor effect comparable to those of the competitor compounds sotorasib in MIA PaCa2 and NCI-H2122, and adagrisib in NCI-H2122. In dose scheduling studies, Compound A given at the same daily dose in a BID or a QD schedule achieved the same response, suggesting an AUC-driven PD/efficacy relationship. In addition to achieving significant efficacy when administered as a single agent, Compound A has the potential to synergize with TNO155, an inhibitor of the phosphatase SHP2. In vitro, JDQ443 in combination with TNO155 was synergistic in the NCI-H2122, HCC-1171 and NCI-H1373 NSCLC cell lines and led to significantly greater cell growth inhibition as compared to JDQ443 alone. In vivo, the combination of JDQ443 with TNO155 significantly improved the sustainability of response and time to relapse seen with JDQ443 as a single agent in Lu99 NSCLC xenografts.

### Example 9: Anti-Tumor Efficacy of Compound A Alone and in Combination with TNO155 in Lu99 KRAS G12C Colorectal Mouse Xenograft Models

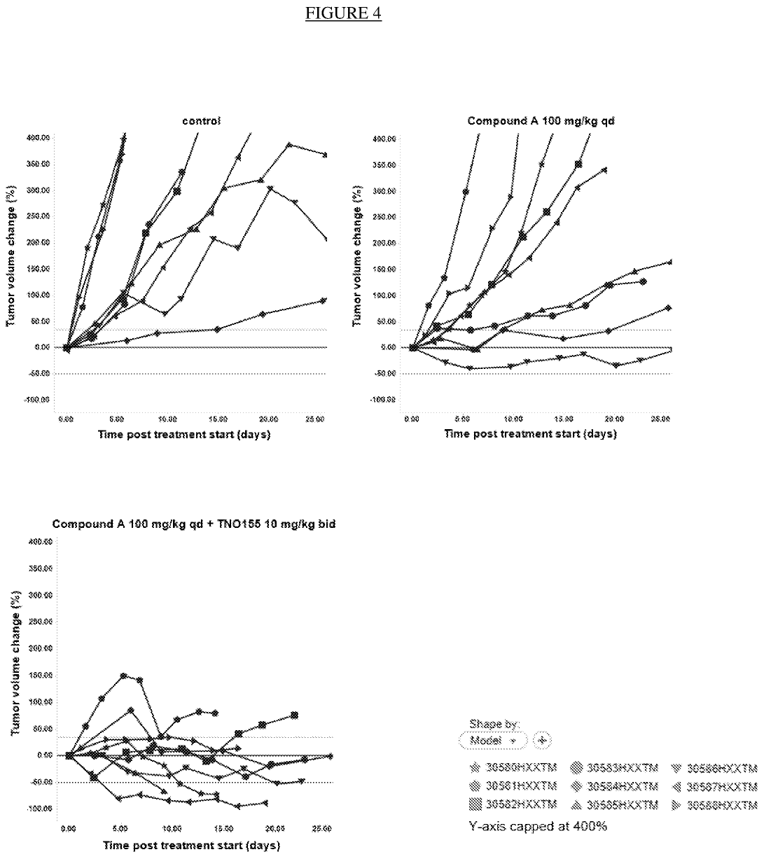

An in vivo combination study of Compound A with TNO155 was conducted in a panel of KRAS G12C-mutated patient-derived xenograft (PDX) models of human colorectal cancer. Female nude mice were implanted subcutaneously with tumor fragments from each PDX model. Individual mice were assigned to treatment groups for dosing once their tumor volume reached 200-250 mm³. One animal per PDX model was assigned to each treatment arm. Mice were left untreated (control) or were treated orally with Compound A at 100 mg/kg daily or a combination of Compound A at 100 mg/kg daily and TNO155 at 10 mg/kg twice daily. The end of study per model was defined as minimum of 28 days treatment, or duration for untreated tumor to reach 1500 mm³, or duration for two doublings of untreated tumor, whichever was slower. Tumor volumes were recorded and are represented as % tumor volume change f SEM for each group. Daily dosing with Compound A led to regression of one PDX model and to a slight to moderate tumor growth delay in some PDX models. The combination of Compound A with TNO155 improved the response in all PDX models, ranging from strong tumor growth inhibition to tumor regression (see FIG. 4).

### Example 10: Compound A in Combination with TNO155 Improves Single-Agent Activity of Compound A, and Compound A can be Reduced while Maintaining Efficacy

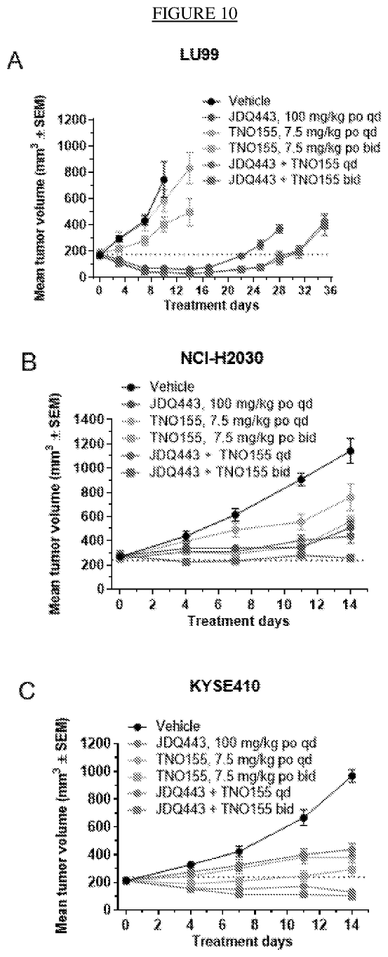

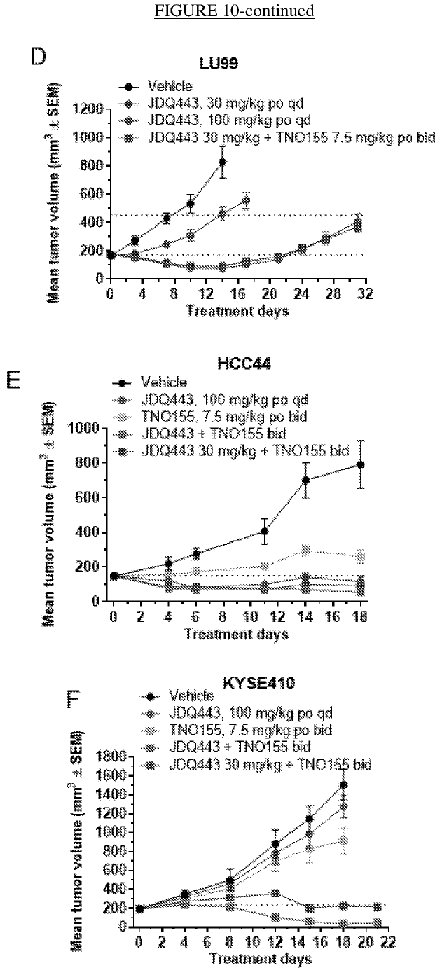

The improved anti-tumor effect of Compound A in combination with the SHP2 inhibitor TNO155 using different dose-regimens was further investigated. Combination studies were conducted with Compound A (100 mg/kg QD) and TNO155 (7.5 mg/kg BID) in three KRAS G12C-mutated tumor models in vivo (LU99, NCI-H2030 and KYSE410). The combination delayed time to relapse of the tumors compared with either single agent in the LU99 model (FIG. 10 “A”) and demonstrated greater antitumor efficacy in H2030 and KYSE410 models (FIG. 10 “B”, “C”, “F”). The effect of the reduced exposure of TNO155 in combination with Compound A in these three xenograft models was also tested. As shown in FIG. 10 “A-C”, TNO155 at 7.5 mg/kg at a twice daily schedule is not necessary in combination with Compound A (100 mg/kg qd) and a once daily schedule is sufficient to maintain efficacy in LU99 and KYSE410 xenografts. The effect of Compound A dose-reduction in combination with TNO155 in LU99. HCC44 and KYSE410 xenografts. In the LU99 xenograft model (FIG. 10 “D”), Compound A at 30 mg/kg daily dose in combination with TNO155 at 7.5 mg/kg bid achieved the same response as Compound A single agent at 100 mg/kg. The efficacy correlated well with target occupancy.

In a corresponding study where KRAS^{G12C} target occupancy was assessed after 5 days treatment of LU99 xenografts with single-agent Compound A at 100 mg/kg qd or in combination with TNO155 at 7.5 mg/kg bid, both regimens achieved comparable reduction of free KRAS^{G12C} in tumors (FIG. 10 “G”). As single-agent, Compound A at 30 mg/kg achieved less reduction of free KRAS^{G12C} in LU99 tumors than at 100 mg/kg, correlating with the dose-dependent single agent anti-tumor efficacy (FIG. 10 “D”). Likewise, in the HCC44 xenograft model (FIG. 10 E), Compound A at 30 mg/kg daily dose in combination with TNO155 at 7.5 mg/kg bid achieved the same response as Compound A single agent at 100 mg/kg. In KYSE410 (FIG. 10 “F”), Compound A at 30 mg/kg in combination with TNO155 at 7.5 mg/kg bid was sufficient to convert a barely responding model to a good responder, achieving stasis. However, Compound A at a daily dose of 100 mg/kg in combination with TNO155 at 7.5 mg/kg bid further improved efficacy and caused tumor regression.

Together, these results indicate that SHP2 blockade can augment the antitumor activity of KRAS^{G12C} inhibitors through enhancing target occupancy establishing a more comprehensive blockade of KRAS-dependent signaling and that a reduced exposure of one of the two drugs maintains the efficacy.

### Example 11: Compound A Potently Inhibits KRAS G12C H950, a Double Mutant Mediating Resistance to Adagrasib in Clinical Trials

GFP-tagged KRASG12C H95Q, KRASG12C Y96D or KRASG12C R68S double mutations were generated by site-directed mutagenesis (QuikChange Lightning Site-Directed Mutagenesis Kit (Catalog #210518) Template: pcDNA3.1(+)EGFP-T2A-FLAG-KRAS G12C and expressed in Cas9 containing Ba/F3 cells by stable transfection. Cells were treated with a dose response curve starting at 10 μM with 1/3 dilution from a 10 mM DMSO stock. Cell lines were treated with indicated compounds for 72 hours and the viabilities of the cells were measured with CellTiter-Glo.

### Results:

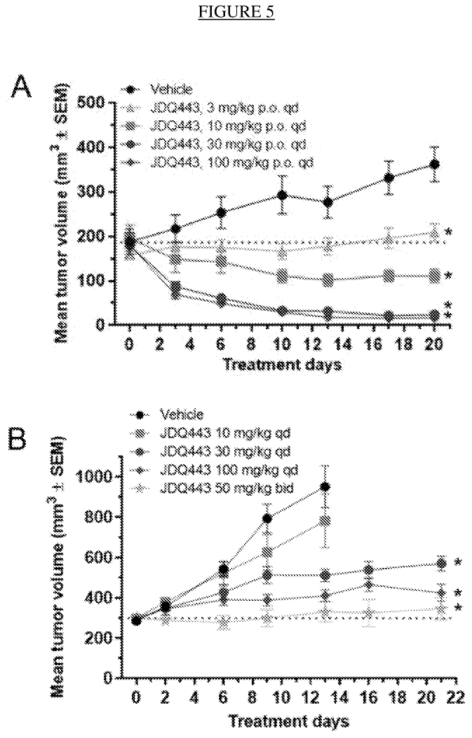

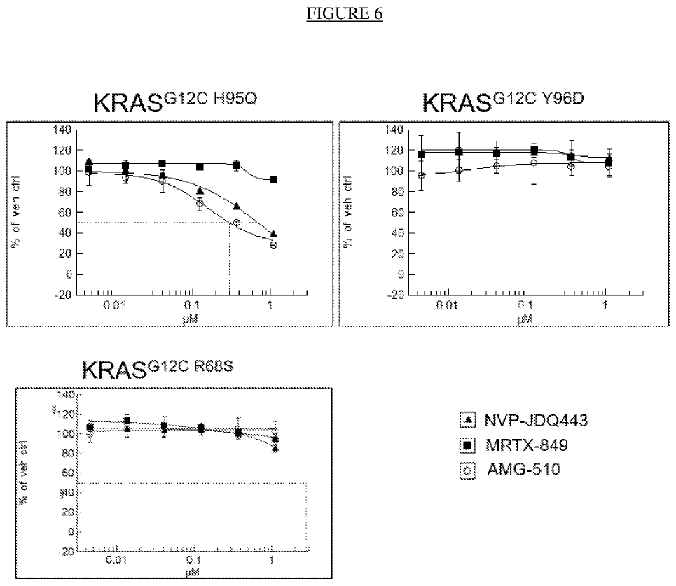

In contrast to MRTX-849 (adagrasib), JDQ443 (Compound A) and AMG-510 (sotorasib) are potently inhibiting the cellular viability of the KRASG12C H95Q double mutant. KRASG12C Y96D or KRASG12C R68S double mutant are not inhibited by MRTX-849, AMG-510 or JDQ443 at the indicated concentrations and in the described setting (Ba/F3 system, 3-day proliferation assay) and confer resistance to all three tested KRASG12C inhibitors. (FIG. 6; y-axis effect of KRASG12C compound in the in vitro proliferation assay in comparison to DMSO control (ie no KRAS G12C compound). The horizontal red dotted line represents the GI50 value.

| | GI₅₀ [μM] +/− standard deviation (STDEV) | | |
|---|---|---|---|
| KRAS G12C inhibitor | KRAS G12C H95Q | KRAS G12C Y96D | KRAS G12C R68S |
| NVP-JDQ443 | 0.57 +/− 0.18 | >10 | >10 |
| (Compound A) | | | |
| AMG-510 (sotorasib) | 0.26 +/− 0.06 | >10 | >10 |
| MRTX-849 (adagrasib) | >10 | >10 | >10 |

In FIG. 6, the uppermost curve (in green) represents treatment with MRTX-849 (adagrasib). The middle curve (in red) represents treatment with NVP-JDQ443 (Compound A). The curve at the bottom (in blue) represents treatment with AMG-510 (sotorasib).

### Conclusion:

Compound A might overcome resistance towards adagrasib in the KRASG12C H95Q setting. In addition, since Compound A has unique binding interactions with mutated KRAS G12C, when compared with sotorasib and adagrasib, Compound A, alone or in combination with one or more therapeutic agent as described herein, may be useful to treat patients suffering from cancer who have previously been treated with other KRAS G12C inhibitors such as sotorasib or adagrasib, or to target resistance after an acquired KRAS resistance mutation emerges on the initial KRAS G12C inhibitor treatment.

### Example 12: Compound A Potently Inhibits KRAS G12C Double Mutants

The effect of Compound A and other KRASG12C inhibitors on second-site mutations reported to confer resistance to adagrasib was also investigated as follows.

### Materials and Methods:

### Cell Lines and KRAS^{G12C} Inhibitors:

The Ba/F3 cell line is a murine pro-B-cell line and is cultured in RPMI 1640 (Bioconcept, #1-41F01-1) supplemented with 10% Fetal Bovine Serum (FBS) (BioConcept, #2-01F30-I), 2 mM Sodium pyurvate (BioConcept, #5-60F00-H), 2 mM stable Glutamine (BioConcept, #5-10K50-H), 10 mM HEPES (BioConcept, #5-31F00-H) and at 37° C. with 5% CO₂, except as otherwise indicated. The parental Ba/F3 cells were cultured in the presence of 5 ng/ml of recombinant murine IL-3 (Life Technologies, #PMC0035). Ba/F3 cells are normally dependent on IL-3 to survive and proliferate, however, by expressing oncogenes they are able to switch their dependency from IL-3 to the expressed oncogene (Curr Opin Oncology, 2007 January; 19(1):55-60. doi: 10.1097/CCO.0b013e328011a25f.)

### Individual Plasmid Mutagenesis and Generation of Ba/F3 Stable Cell Lines:

QuikChange Lightning Site-Directed Mutagenesis Kit (Agilent; #210519) was used to generate the resistant mutations on the pSG5_Flag-(codon optimized) KRAS^{G12C}_puro plasmid template and sequences were confirmed by sanger sequencing.

| Primer | Primer sequence | SEQ ID NO |
|---|---|---|
| H95R_forward | 5′-gtcatttgaagatatccaccgttatcgogagcagattaaga-3′ | 552 |
| H95R_reverse | 5′-tcttaatctgctcgcgataacggtggatatcttcaaatgac-3′ | 553 |
| H95Q_forward | 5′-tcatttgaagatatccaccagtatcgcgagcagattaagag-3′ | 554 |
| H95Q_reverse | 5′-ctcttaatctgctogcgatactggtggatatcttcaaatga-3′ | 555 |
| H95D_forward | 5′-agtcatttgaagatatccacgattatogcgagcagattaag-3′ | 556 |
| H95D_reverse | 5′-cttaatctgctcgcgataatcgtggatatcttcaaatgact-3′ | 557 |
| MR68S_forward | S′-gaagagtactccgcaatgagcgatcaatacatgaggacg-3′ | 558 |
| R68S_reverse | 5′-cgtcctcatgtattgatcgctcattgcggagtactcttc-3′ | 559 |
| Y96C_forward | 5′-cgaagtcatttgaagatatccaccattgtcgcgagcagatta-3′ | 560 |
| Y96C_reverse | 5′-taatctgctcgcgacaatggtggatatcttcaaatgacttcg-3′ | 561 |
| Y96D_forward | 5′-cgaagtcatttgaagatatccaccatgatcgcgagcagatt-3 | 562 |
| Y96D_reverse | 5′-aatctgctcgcgatcatggtggatatcttcaaatgacttog-3′ | 563 |

The mutant plasmids were transfected into the Ba/F3 WT cells by electroporation with the NEON transfection kit (Invitrogen, #MPK10025). Therefore, two million Ba/F3 cells have been electroporated with 10 μg pf plasmids with the NEON System (Invitrogen, #MPK5000), using following conditions Voltage (V) 1635, Width (ms) 20, Pulses 1. After 72 h of electroporation, puromycin selection was performed at 1 μg/ml to generate stable cell lines.

### IL-3 Withdrawal

Ba/F3 cells are normally dependent on IL-3 to survive and proliferate, however, by expressing oncogenes they are able to switch their dependency from IL-3 to the expressed oncogene. To assess whether the KRAS^{G12C} single and double mutants are able to sustain the proliferation of Ba/F3 cells, the engineered Ba/F3 cells expressing the mutant constructs were cultured in absence of IL-3. Cell number and viability was measured every three days and after seven days the IL-3 withdrawal was completed. The expression of the mutants after the IL-3 withdrawal were confirmed by Western Blot (data not shown, an upwards shift was observed for KRAS^{G12C/R68S}).

### Drug Response Curves for KRASG12C Inhibitors and Validation of Resistance Mutations:

1000 Ba/F3 cells/well were seeded at in 96-well plates (Greiner Bio-One, #655098). Treatment was performed on the same day with a Tecan D300e drug dispenser. Viability was detected on the same day of treatment for the start plate (Day 0) and three days post-treatment (Day 3) using the CellTiter-Glo Luminescent Cell Viability Assay (Promega, #G7573) on a Tecan infinitiy M200 Pro reader (Intergration Time 1000 ms).

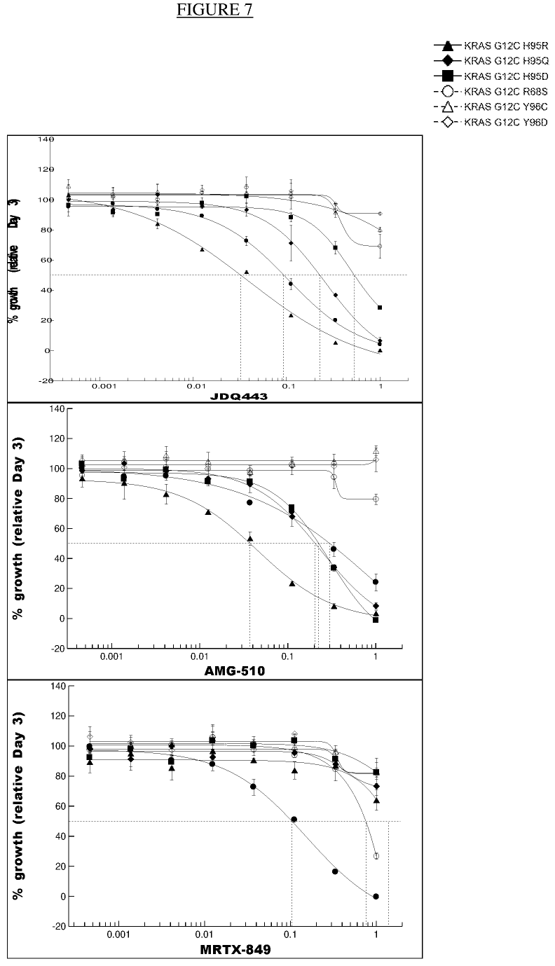

To determine the growth, the three days post-treatment (Day 3) readout was normalized to start plate (Day 0). The percentage viability was then calculated by normalizing treated wells to DMSO treated control samples. XLfit was used to make the fitted curve with a Sigmoidal Dose-Response Model (four-parameter curve) (FIG. 7). The horizontal red dotted line represents the GI50 value. Tabular data are shown below.

TABLE
Effect of Compound A (JDQ443) on the the proliferation of KRAS G12C/H95 double
mutants. («STDEV» indicates the standard deviation for the % growth value)

| % growth of | | | | | | | | |
|---|---|---|---|---|---|---|---|---|
| treated cells | | | | | | | | |
| relative to day | conc [μM] | | | | | | | |
| 3 treatment | 1 | 0.333333 | 0.111111 | 0.037037 | 0.012346 | 0.004115 | 0.001372 | 0.000457 |
| G12C | 4.279 | 20.252 | 44.204 | 72.785 | 89.361 | 93.832 | 97.516 | 95.501 |
| G12C | 0.961 | 0.345 | 3.567 | 3.058 | 1.072 | 0.770 | 3.921 | 3.639 |
| STDEV | | | | | | | | |
| H95R | 0.321 | 5.323 | 23.425 | 52.178 | 66.971 | 83.996 | 92.517 | 103.118 |
| H95R | 0.943 | 0.276 | 0.779 | 1.034 | 0.897 | 3.344 | 3.811 | 7.044 |
| STDEV | | | | | | | | |
| H95Q | 6.635 | 36.908 | 71.333 | 93.319 | 95.722 | 103.375 | 93.606 | 100.054 |
| H95Q | 2.025 | 0.910 | 11.656 | 4.209 | 0.919 | 6.685 | 3.996 | 2.621 |
| STDEV | | | | | | | | |
| H95D | 28.569 | 68.199 | 88.358 | 102.308 | 97.783 | 90.379 | 91.567 | 96.429 |
| H95D | 1.055 | 4.247 | 2.997 | 6.409 | 3.644 | 0.087 | 2.074 | 7.212 |
| STDEV | | | | | | | | |
| R68S | 69.159 | 93.111 | 103.721 | 108.276 | 104.608 | 100.329 | 103.390 | 100.931 |
| R68S | 7.892 | 4.607 | 9.627 | 6.839 | 0.781 | 4.288 | 2.838 | 0.996 |
| STDEV | | | | | | | | |
| Y96C | 80.229 | 91.765 | 102.592 | 104.222 | 96.167 | 105.515 | 102.424 | 109.116 |
| Y96C | 1.667 | 0.222 | 2.981 | 1.230 | 0.896 | 4.837 | 5.863 | 4.380 |
| STDEV | | | | | | | | |
| Y96D | 90.864 | 97.260 | 105.866 | 103.946 | 106.613 | 98.191 | 102.224 | 100.903 |
| Y96D | 0.852 | 0.105 | 4.943 | 6.090 | 3.050 | 1.435 | 5.305 | 3.229 |
| STDEV | | | | | | | | |

TABLE
Effect of sotorasib (AMG510) on the the proliferation of KRAS G12C/H95 double mutants
(«STDEV» indicates the standard deviation for the % growth value)

| % growth of | | | | | | | | |
|---|---|---|---|---|---|---|---|---|
| treated cells | | | | | | | | |
| relative to day | conc [μM] | | | | | | | |
| 3 treatment | 1 | 0.333333 | 0.111111 | 0.037037 | 0.012346 | 0.004115 | 0.001372 | 0.000457 |
| G12C | 24.244 | 46.199 | 71.315 | 77.193 | 92.241 | 95.211 | 94.786 | 100.303 |
| G12C | 5.785 | 4.703 | 0.727 | 0.776 | 2.500 | 1.603 | 2.016 | 5.046 |
| STDEV | | | | | | | | |
| H95R | 3.485 | 8.354 | 23.503 | 53.471 | 71.098 | 83.029 | 90.450 | 93.527 |
| H95R | 1.825 | 1.731 | 0.971 | 4.408 | 1.615 | 6.613 | 10.693 | 5.561 |
| STDEV | | | | | | | | |
| H95Q | 8.566 | 34.323 | 68.123 | 89.685 | 93.396 | 98.559 | 103.616 | 98.858 |
| H95Q | 2.203 | 1.572 | 6.416 | 5.515 | 0.970 | 1.603 | 4.610 | 1.842 |
| STDEV | | | | | | | | |
| H95D | −1.171 | 33.638 | 74.059 | 91.299 | 91.484 | 99.189 | 93.064 | 103.216 |
| H95D | 0.374 | 1.962 | 1.683 | 0.716 | 2.837 | 6.975 | 2.489 | 5.237 |
| STDEV | | | | | | | | |
| R68S | 79.748 | 94.396 | 102.811 | 98.842 | 99.936 | 100.994 | 94.166 | 95.566 |
| R68S | 3.473 | 7.672 | 2.021 | 3.308 | 0.455 | 0.885 | 1.904 | 1.680 |
| STDEV | | | | | | | | |
| Y96C | 111.964 | 105.031 | 103.341 | 97.712 | 104.892 | 109.010 | 106.167 | 103.355 |
| Y96C | 3.326 | 0.058 | 2.472 | 2.258 | 0.105 | 2.374 | 0.266 | 3.889 |
| STDEV | | | | | | | | |
| Y96D | 106.099 | 101.660 | 101.868 | 97.311 | 102.190 | 106.308 | 101.134 | 105.511 |
| Y96D | 7.943 | 8.231 | 5.850 | 1.065 | 8.679 | 8.652 | 10.362 | 3.819 |
| STDEV | | | | | | | | |

TABLE
Effect of adagrasib (MRTX-849) on the the proliferation of KRAS G12C/H95 double
mutants («STDEV» indicates the standard deviation for the % growth value)

| % growth of | | | | | | | | |
|---|---|---|---|---|---|---|---|---|
| treated cells | | | | | | | | |
| relative to day | conc [μM] | | | | | | | |
| 3 treatment | 1 | 0.333333 | 0.111111 | 10.037037 | 0.012346 | 0.004115 | 0.001372 | 0.000457 |
| G12C | −0.266 | 16.329 | 51.013 | 72.820 | 87.908 | 90.538 | 97.358 | 99.591 |
| G12C | 0.026 | 0.281 | 1.081 | 5.419 | 4.200 | 3.922 | 1.669 | 7.272 |
| STDEV | | | | | | | | |
| H95R | 64.097 | 87.126 | 83.910 | 90.693 | 96.751 | 85.570 | 95.027 | 89.422 |
| H95R | 6.732 | 10.191 | 5.867 | 1.326 | 1.261 | 8.143 | 11.049 | 7.126 |
| STDEV | | | | | | | | |
| H95Q | 73.397 | 88.044 | 95.662 | 101.071 | 92.756 | 100.136 | 91.377 | 98.312 |
| H95Q | 6.148 | 0.323 | 0.148 | 0.289 | 1.657 | 1.053 | 5.052 | 1.475 |
| STDEV | | | | | | | | |
| H95D | 82.356 | 91.214 | 103.587 | 100.461 | 103.684 | 89.514 | 98.169 | 92.404 |
| H95D | 6.938 | 2.123 | 4.434 | 4.185 | 5.798 | 3.766 | 1.029 | 2.848 |
| STDEV | | | | | | | | |
| R68S | 26.823 | 84.668 | 94.008 | 101.452 | 105.903 | 100.837 | 99.142 | 97.443 |
| R68S | 2.129 | 0.233 | 1.666 | 0.085 | 7.640 | 1.368 | 3.217 | 1.746 |
| STDEV | | | | | | | | |
| Y96C | 82.638 | 96.520 | 99.562 | 102.615 | 105.187 | 101.092 | 100.220 | 99.819 |
| Y96C | 9.540 | 4.002 | 2.065 | 3.987 | 4.264 | 2.673 | 6.930 | 10.013 |
| STDEV | | | | | | | | |
| Y96D | 81.671 | 95.056 | 108.171 | 97.824 | 105.964 | 97.437 | 102.545 | 106.432 |
| Y96D | 3.884 | 2.842 | 0.058 | 4.085 | 8.415 | 7.575 | 0.349 | 6.625 |
| STDEV | | | | | | | | |

### Western Blot

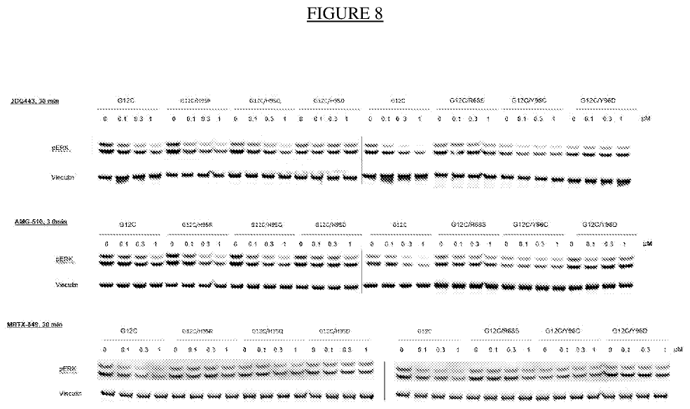

After treatment with the different compounds at the indicated concentrations and for the indicated time, the cells were collected, pelleted and snap frozen at −80′° C. Sixty L of lysis buffer (50 mM Tris HCl, 120 mM NaCl, 25 mM NaF, 40 mM β-glycerol phosphate disodium salt pentahydrate, 1% NP40, 1 μM microcystin, 0.1 mM Na3VO3, 0.1 mM PMSF, 1 mM DTT and 1 mM benzamidine, supplemented with 1 protease inhibitor cocktail tablet (Roche) for 10 mL of buffer) was added to each sample. The samples were then vortexed, incubated on ice for 10 min, vortexed again and centrifuged at 14000 rpm at 4° C. for 10 min. Protein concentration was determined with the BCA Protein Assay kit (Pierce, 23225). After normalization to the same total volume with lysis buffer, NuPAGE™ LDS Sample buffer 4× (Invitrogen, NP0007) and NuPAGE™ Sample reducing agent 10× (Invitrogen, NP0009) was added. The samples were heated at 70° C. for 10 min before loading on a NuPAGE™ Novex™ 4-12% Bis-Tris Midi Protein Gel, 26-wells (Invitrogen, WG11403A). Gels were run for 45 min at 200 V (PowerPac HC, Biorad) in NuAGE MES SDS running buffer (Invitrogen, NP0002). The proteins were transferred for 7 min at 135 mA per gel on a Trans-Blot® Turbo™ Midi Nitrocellulose Transfer Packs membrane (Biorad, 1704159) using the Trans-Blot® Turbo™ system (Biorad) before staining the membrane with Ponceau red (Sigma, P7170). The membranes were blocked with TBST with 5% of milk at RT. Anti-RAS (Abcam, 108602) and anti-phospho-ERK 1/2 p44/42 MAPK (Cell Signaling, 4370) antibodies were incubated overnight at 4° C., the anti-vinculin (Sigma, V9131) antibody was incubated for 1 h at RT. Membranes were washed 3× for 5 min with TBST and the anti-rabbit (Cell Signaling, 7074) and anti-mouse (Cell Signaling, 7076) secondary antibodies were incubated for 1 h at RT. All antibodies were diluted in TBST to 1/1000, except of anti-vinculin (1/3000). Revelation was performed with WesternBright ECL (Advansta, K-12045-D20) for Ras and vinculin and with SuperSignal West Femto maximum sensitivity substrate (Thermo Fischer, 34096), on a Fusion FX (Vilber Lourmat) using the FusionCapt Advance FX7 software. (FIG. 8).

### Results

TABLE
Compound A (JDQ443) inhibits the proliferation of KRAS G12C/H95
double mutants. Ba/F3 cells expressing the indicated
FLAG-KRAS^{G12C} single or double mutants were treated with
JDQ443 (Compound A, AMG-510 (sotorasib) and MRTX-849 (adagrasib)
(8-point dilution starting at 1 mM) for 3 days and the inhibtion
of proliferation was assessed by Cell titer glo viability assay.
The average of GI₅₀ ± standard deviation (St DV) of
4 independent experiments are shown.

| GI50 ± St DV [μM] | JDQ443 | AMG-510 | MRTX-849 |
|---|---|---|---|
| G12C | 0.115 ± 0.060 | 0.389 ± 0.235 | 0.136 ± 0.071 |
| G12C/H95R | 0.024 ± 0.006 | 0.033 ± 0.008 | >1 |
| G12C/H95Q | 0.284 ± 0.041 | 0.233 ± 0.022 | >1 |
| G12C/H95D | 0.612 ± 0.151 | 0.262 ± 0.088 | >1 |
| G12C/R68S | >1 | >1 | 0.707 ± 0.165 |
| G12C/Y96C | >1 | >1 | >1 |
| G12C/Y96D | >1 | >1 | >1 |

### Biophysical Data

### Material and Methods:

### Preparation of Reagents:

### Cloning, Expression and Purification of RAS Protein Constructs

The E. coli expression constructs used in this study were based on the pET system and generated using standard molecular cloning techniques. Following the cleavable N-terminal his affinity purification tag the cDNA encoding KRAS, NRAS, and HRAS comprised aa 1-169 and was codon-optimized and synthesized by GeneArt (Thermo Fisher Scientific). Point mutations were introduced with the QuikChange Lightning Site-Directed Mutagenesis kit (Agilent). All final expression constructs were sequence verified by Sanger sequencing.

Two liters of culture medium were inoculated with a pre-culture of E. coli BL21(DE3) freshly transformed with the expression plasmid and protein expression induced with 1 mM isopropyl-β-D-thiogalactopyranoside (Sigma) for 16 hours at 18° C. Proteins with an avi-tag were transformed into E. coli harboring a compatible plasmid expressing the biotin ligase BirA and the culture medium was supplemented with 135 μM d-biotin (Sigma).

Cell pellets were resuspended in buffer A (20 mM Tris, 500 mM NaCl, 5 mM imidazole, 2 mM TCEP, 10% glycerol, pH 8.0) supplemented with Turbonuclease (Merck) and cOmplete protease inhibitor tablets (Roche). The cells were lysed by three passages through a homogenizer (Avestin) at 800-1000 bar and the lysate clarified by centrifugation at 40000 g for 40 min. The lysate was loaded onto a HisTrap HP 5 ml column (Cytiva) mounted on an ÅKTA Pure 25 chromatography system (Cytiva). Contaminating proteins were washed away with buffer A and bound protein was eluted with a linear gradient to buffer B (buffer A supplemented with 200 mM imidazole). During dialysis O/N the N-terminal His affinity purification tags on the non-tagged and avi-tagged proteins were cleaved off by TEV or HRV3C protease, respectively. The protein solution was re-loaded onto a HisTrap column and the flow through containing the target protein collected. Guanosine 5′-diphosphate sodium salt (GDP, Sigma) or GppNHp-Tetralithium salt (Jena Bioscience) was added to a 24-32× molar excess over protein. EDTA (pH adjusted to 8) was added to a final concentration of 25 mM. After 1 hour at room temperature the buffer was exchanged on a PD-10 desalting column (Cytiva) against 40 mM Tris, 200 mM (NH4)2SO4, 0.1 mM ZnCl2, pH 8.0. GDP (for KRAS G12C resistance mutants H95Q/D/R, Y96D/C and R68S) or GppNHp was added to a 24-32× molar excess over protein to the eluted protein. 40 U Shrimp Alkaline Phosphatase (New England Biolabs) was added to GppNHp containing samples only. The sample was then incubated for 1 hour at 5° C. Finally, MgCl2 was added to a concentration of about 30 mM. The protein was then further purified over a HiLoad 16/600 Superdex 200 μg column (Cytiva) pre-equilibrated with 20 mM HEPES, 150 mM NaCl, 5 mM MgCl2, 2 mM TCEP, pH 7.5.

The purity and concentration of the protein was determined by RP-HPLC, its identity was confirmed by LC-MS. Present nucleotide was determined by ion-pairing chromatography [Eberth et al, 2009].

### Determination of Covalent Rate Constants by RapidFire MS

### Assay and Curve Fitting

Serial dilutions of the test compounds (50 μM, 2 dilutions) were prepared in 384 well plates and incubated with 1 μM KRAS G12C (with/without additional mutants) in 20 mM Tris pH7.5, 150 mM NaCl, 100 μM MgCl₂, 1% DMSO at room temperature. Reactions were stopped at different time points by addition of formic acid to 1%. MS measurements were carried out using a Agilent 6530 quadrupole time-of-flight (QToF) MS system coupled to an Agilent RapidFire autosampler RF360 device, resulting in % modification values for each well. In parallel, compound solubility was assessed by nephelometry and compound concentrations resulting in measurable turbidity were excluded from curve fitting.

Plotting the % modification vs. time allowed for extraction of k_{obs} values for the different compound concentrations. In a second step, the obtained k_{obs} values were plotted against the compound concentrations. Rate constants (i.e. kᵢₙₙₑᵣ/K_{I}) were derived from the initial linear part of the resulting curves.

### MS Measurements

The RapidFire autosampler RF 360 was used to perform the injections. Solvents were delivered by Agilent 1200 pumps. A C18 Solid Phase Extraction (SPE) cartridge was used for all experiments.

A volume of 30 μL was aspirated from each well of a 384-well plate. The sample load/wash time was 3000 ms at a flow rate of 1.5 mL/min (H2O, 0.1% formic acid); elution time was 3000 ms (acetonitrile, 0.1% formic acid); reequilibration time was 500 ms at a flow rate of 1.25 mL/min (H2O, 0.1% formic acid).

Mass spectrometry (MS) data were acquired on an Agilent 6530 quadrupole time-of-flight (QToF) MS system, coupled to a dual Electrospray (AJS) ion source, in positive mode. The instrument parameters were as follows: gas temperature 350° C., drying gas 10 L/min, nebulizer 45 psi, sheath gas 350° C., sheath gas flow 11 L/min, capillary 4000 V, nozzle 1000 V, fragmentor 250 V, skimmer 65 V, octapole RF 750 V. Data were acquired at the rate of 6 spectra/s. The mass calibration was performed over the 300-3200 m/z range.

All data processing was performed using a combination of Agilent MassHunter Qualitative Analysis, Agilent Rapid-Fire control software, and the Agilent DA Reprocessor Offline Utilities. A Maximum Entropy algorithm produced zero-charge spectra in separate files per injection. A batch processing generated a single file incorporating all mass spectra in a text format as x,y coordinates. This file was used to calculate the % of protein modification in each well.

### Results

Quantification of the second order rate constants for modification for the indicated constructs (all GDP-loaded) was carried out using kinetic MS experiments, measuring % modification at different time points for a range of compound concentrations. Kᵢₙₙₑᵣ/K_{I} was extrapolated from the initial slope of the k_{obs} vs. compound concentration plot. Activities against KRAS G12D:GDP were set to 1 and relative activities for the resistance mutants are given. Average values of n=4 experiments for KRAS G12C, n=3 for G12C_Y96D and n=2 for other mutants are given in the Table below.

TABLE
Fold change of second order rate constant (K_{inact}/K_{I}) for resistance mutants relative to KRAS
G12C

| KRAS mutant | | | | | | | |
|---|---|---|---|---|---|---|---|
| (GDP) | G12C | G12C_H95R | G12C_H95Q | G12C_H95D | G12C_R68S | G12C_Y96C | G12C_Y96D |
| Compound A | 1 | 1.04 | 0.40 | 0.20 | 0.14 | 0.03 | 0.004 |
| (JDQ443) | | | | | | | |
| Sotorasib | 1 | 2.39 | 1.67 | 1.45 | 0.31 | <0.002 | <0.001 |
| Adagrasib | — | <0.05 | <0.05 | <0.05 | 0.38 | <0.002 | <0.001 |

Quantification of the second order rate constants for modification for the indicated constructs (all GDP-loaded) was carried out using kinetic MS experiments, measuring % modification at different time points for a range of compound concentrations. Kᵢₙₙₑᵣ/KI was extrapolated from the initial slope of the K_{obs} vs. compound concentration plot. Average values of n=4 experiments for KRAS G12C, n=3 for G12C_Y96D and n=2 for other mutants are given.

TABLE
Second order rate constants (K_{inact}/KI [mM−1 * s−1]) for Compound A (JDQ443), sotorasib and
adagrasib against resistance mutants

| KRAS mutant | | | | | | | |
|---|---|---|---|---|---|---|---|
| (GDP) | G12C | G12C_H95R | G12C_H95Q | G12C_H95D | G12C_R68S | G12C_Y96C | G12C_Y96D |
| Compound A | 24.3 | 25 | 9.5 | 4.85 | 3.45 | 0.65 | 0.09 |
| (JDQ443) | | | | | | | |
| Sotorasib | 9 | 21.5 | 15 | 13.05 | 2.75 | <0.02 | <0.01 |
| Adagrasib | 26 | <1 | <1 | <1 | 9.9 | <0.04 | <0.01 |

### Conclusions

First generation KRAS G12C inhibitors have shown efficacy in clinical trials. However, the emergence of mutations that disrupt inhibitor binding and reactivation in downstream pathways, limits the duration of response. Second-site mutants reported to confer resistance to adagrasib in clinical trials (ref: N Engl J Med. 2021 Jun. 24; 384(25):2382-2393. doi: 10.1056/NEJMoa2105281., Cancer Discov. 2021 August; 1(8):1913-1922. doi: 10.1158/2159-8290.CD-21-0365. Epub 2021 Apr. 6.PMID: 33824136.) were expressed in Ba/F3 cells and analyzed for their sensitivity towards Compound A (JDQ443) in comparison to KRAS G12C (GI₅₀=0.115 t 0.060 mM). As expected from the binding mode. Compound A inhibited proliferation and signaling of KRAS G12C H95 double mutants. Compound A potently inhibited the proliferation of G12C/H95R and G12C/H95Q (GI₅₀=0.024±0.006 mM, GI₅₀=0.284±0.041 mM, respectively), while expression of G12C/R68S, G12C/Y96C and G12C/Y96D conferred resistance to Compound A (GI₅₀>1 mM, all).

Surprisingly, expression of G12C/H95D resulted in reduced sensitivity to Compound A (GI₅₀=0.612±0.151 mM) compared to H95R or Q although Compound A is not directly interacting with Histidine 95. Western blot analysis of pERK upon Compound A treatment as well as the analysis of the rate constants of Compound A (biophysical data, above) towards these clinically observed SWII pocket mutations in biophysical settings were in agreement with the cellular growth inhibition data (see table).

The difference between H95D compared to H95R or Q could be due the negative charge of the aspartate, which could further increase the negative electrostatic potential of the KRAS G12C surface. This might affect ligand recognition and therefore decrease the specific reactivity and cellular activity of Compound A for this mutant. Another possible explanation is that the H95D mutation could affect KRAS dynamic so that the conformation allowing Compound A binding becomes less accessible.

In conclusion, the data show Compound A should overcome adagrasib induced resistance in G12C/Q95R or G12C/H95Q settings. Compound A treatment, particularly in combinations of the invention may still be useful in the G12C/H95Q setting where it has shown activity.

### Example 13: Study of Compound A in Patients with Advanced Solid Tumors Harboring the KRAS G12C Mutation

A study to assess the safety and tolerability of Compound A single agent, of Compound A in combination with TNO155, of Compound A in combination with a PD1-inhibitor (such as spartalizumab or tislelizumab), and of Compound A in combination with TNO155 and a PD1-inhibitor (such as spartalizumab or tislelizumab), and to identify the maximum tolerated dose and/or recommended dose and regimen for future studies is carried out. The study is also carried out to evaluate the anti-tumor activity of the study treatments and to evaluate the immunogenicity of spartalizumab or tislelizumab when dosed in combination with Compound A and/or TNO155.

The study is conducted in adult patients with advanced solid tumors who harbor the KRAS G12C mutation. In expansion, advanced (metastatic or unresectable) non-small cell lung cancer patients who harbor the KRAS G12C mutation and who are in the second or third line treatment setting will be enrolled. Additional groups of advanced colorectal cancer patients who have the KRAS G12C mutation and who have failed standard of care therapy (i.e. fluropyrimidine-, oxaliplatin-, and/or irinotecan-based chemotherapy) will also be enrolled in the Compound A single agent and Compound A plus TNO155 expansion groups.

Compound A is administered orally (p.o.) QD or BID continuously on a 21-day cycle. In embodiments of the invention, Compound A. or a pharmaceutically acceptable salt, solvate or hydrate thereof, Compound A, or a pharmaceutically acceptable salt, solvate or hydrate thereof, is administered at a therapeutically effective dose ranging from 200 to 1600 mg per day, e.g. from 400 to 1600 m per day. For example, the total daily dose of Compound A may be selected from 200, 300, 400, 600, 800, 1000, 1200 and 1600 mg. The total daily dose of Compound A may be administered continuously, on a QD (once a day) or BID (twice a day) regimen.

Compound A may be administered at a total daily dose of 100 mg to 400 mg, e.g. 200 to 200 mg. In particular, it may be administered at a total daily dose of 400 mg, administered once daily or twice daily. It may also be administered at a total daily dose of 200 mg, administered once daily or twice daily.

It may also be administered at a total daily dose of 600 mg, administered once or twice daily.

TNO155 is administered p.o. QD or BID in a 2 week on/1 week off schedule or continuously.

TNO155 may be administered at a total daily dose ranging from 10 to 80 mg, or from 10 to 60 mg. For example, the total daily dose of TNO155 may be selected from 10, 15, 20, 30, 40, 60 and 80 mg. For example, TNO may be dosed at a total daily dose of 10, 15, 20 or 30 mg.

The total daily dose of TNO155 may be administered continuously, QD (once a day) or BID (twice a day) on QD or BID on a 2 weeks on/l week off schedule. The total daily dose of TNO155 may be administered continuously, QD (once a day) or BID (twice a day) on QD or BID on continuously (i.e. without a drug holiday).

For example, TNO155 may be administered at a total daily dose of 20 mg, administered once or twice a day, either continuously, or on a drug holiday schedule such as a 2 week on/I week off schedule.

If spartalizumab is used as the PD1-inhibitor, it is administered intravenously on a 21-day cycle at a dose of about 300 mg once every 3 weeks, or at a dose of about 400 mg once every 4 weeks.

If tislelizumab is used as the PD1-inhibitor, it is administered intravenously on a 21-day cycle at a dose of about 200 mg once every 3 weeks, or at a dose of about 300 mg once every 4 weeks.

Other doses and dosing regimens as described in the description may also be used.

Efficacy of the therapeutic methods of the invention may be determined by methods well known in the art, e.g. determining Best Overall Response (BOR), Overall Response Rate (ORR), Duration of Response (DOR), Disease Control Rate (DCR), Progression Free Survival, (PFS) and Overall Survival (OS) per RECIST v.1.1.

| | |
|---|---|
| Key Inclusion criteria | Dose Escalation: |
| | Patients with advanced (metastatic or unresectable) KRAS G12C |
| | mutated solid tumors who have received and failed standard of |
| | care therapy or are intolerant or ineligible to approved therapies. |
| | Dose Expansion: |
| | Advanced (metastatic or unresectable) KRAS G12C mutated non- |
| | small cell lung cancer patients who are in the second or third line |
| | treatment setting who have received and failed a platinum-based |
| | chemotherapy regimen and immune checkpoint inhibitor therapy, |
| | unless patient refused or was ineligible to receive such therapy |
| | Advanced (metastatic or unresectable) KRAS G12C mutated |
| | colorectal cancer patients who have received and failed standard- |
| | of-care therapy, including a fluropyrimidine-, oxaliplatin-, and/ |
| | or irinotecan-based chemotherapy, unless patient refused or was |
| | ineligible to such therapy. |
| | All Patients: |
| | ECOG performance status of 0 or 1. |
| | Patients must have a site of disease amenable to biopsy and be a |
| | candidate for tumor biopsy according to the institution's own |
| | guidelines and requirements for such procedures. |
| Key Exclusion criteria | Tumors harboring driver mutations that have approved therapies |
| | or tumors with known activating KRAS, NRAS, HRAS, BRAF, or |
| | PTPN11 (SHP2) mutations, with the exception of KRAS G12C |
| | mutations. |
| | Prior treatment with a KRAS G12C inhibitor will be exclusionary |
| | for patients in the single agent dose escalation arm and all patients |
| | in dose expansion. |
| | Prior treatment with a SHP2 inhibitor is not allowed for NSCLC |
| | patients enrolled into the dose expansion parts, in the Compound |
| | A single agent and Compound A plus TNO155 expansion groups. |
| | Active brain metastases, i.e. symptomatic brain metastases or |
| | known leptomeningeal disease |
| | Clinically significant cardiac disease or risk factors at screening |
| | Insufficient bone marrow, hepatic or renal function at screening: |
| Efficacy assessments | Best Overall Response (BOR), Overall Response Rate (ORR), |
| | Duration of Response (DOR), Disease Control Rate (DCR), |
| | Progression Free Survival, (PFS) and Overall Survival (OS) per |
| | RECIST v.1.1 |

The clinical trial is ongoing. Based on preliminary data, Compound A treatment has shown an acceptable safety and tolerability profile and has shown early signs of clinical efficacy.

### Example 14: Clinical Study of Compound A as Monotherapy in Participants with Locally Advanced or Metastatic KRAS G12C Mutant Non-Small Cell Lung Cancer

An open label study which is designed to compare Compound A as monotherapy to docetaxel in participants with advanced non-small cell lung cancer (NSCLC) harboring a KRAS G12C mutation who have been previously treated with a platinum-based chemotherapy and immune checkpoint inhibitor therapy either in sequence or in combination may be carried out.

The study consists of 2 parts:
Randomized part will evaluate the efficacy and safety of Compound A as monotherapy in comparison with docetaxel.
Extension part will be open after final progression-free survival (PFS) analysis (if the primary endpoint has met statistical significance) to allow participants randomized to docetaxel treatment to crossover to receive Compound A treatment.

The study population include adult participants with locally advanced or metastatic (stage IIIB/IIIC or IV) KRAS G12C mutant non-small cell lung cancer who have received prior platinum-based chemotherapy and prior immune checkpoint inhibitor therapy administered either in sequence or as combination therapy.

Participants are treated with Compound A or docetaxel following local guidelines as per standard of care and product labels (docetaxel concentrated solution for infusion, intravenously administered)

### Primary Outcome Measures Include:

Progression free survival (PFS)
PFS is the time from date of randomization/start of treatment to the date of event defined as the first documented progression or death due to any cause. PFS is based on central assessment and using RECIST 1.1 criteria.

### Secondary Outcome Measures Include:

Overall Survival (OS)
OS is defined as the time from date of randomization to date of death due to any cause
Overall Response Rate (ORR)
ORR is defined as the proportion of patients with best overall response of complete response (CR) or partial response (PR) based on central and local investigator's assessment according to RECIST 1.1.
Disease Control Rate (DCR)
DCR is defined as the proportion of participants with Best Overall Response (BOR) of Complete Response (CR), Partial Response (PR), Stable Disease (SD) or Non-CR/Non-PD.
Time To Response (TTR)
TTR is defined as the time from the date of randomization to the date of first documented response (CR or PR, which must be confirmed subsequently)
Duration of Response (DOR)
DOR is calculated as the time from the date of first documented response (complete response (CR) or partial response (PR)) to the first documented date of progression or death due to underlying cancer.
Progression-Free Survival after next line therapy (PFS2)
PFS2 (based on local investigator assessment) is defined as time from date of randomization to the first documented progression on next line therapy or death from any cause, whichever occurs first.
Concentration of Compound A and its metabolite in plasma
To characterize the pharmacokinetics of Compound A and its metabolite HZC320
Time to definitive deterioration of Eastern Cooperative Group of Oncology Group (ECOG) performance status
Deterioration of Eastern Cooperative Oncology Group (ECOG) Performance Status (PS)
Time to definitive 10-point deterioration symptom scores of chest pain, cough and dyspnea per QLQ-LC13
The EORTC QLQ LCl3 is a 13-item, lung cancer specific questionnaire module, and it comprises both multi-item and single-item measures of lung cancer-associated symptoms (i.e. coughing, hemoptysis, dyspnea and pain) and side-effects from conventional chemo- and radiotherapy (i.e. hair loss, neuropathy, sore mouth and dysphagia). The time to definitive 10-point deterioration is defined as the time from the date of randomization to the date of event, which is defined as at least 10 points absolute increase from baseline (worsening), with no later change below the threshold or death due to any cause
Time to definitive deterioration in global health status/QoL, shortness of breath and pain per QLQ-C30
The EORTC QLQ-C30 is a questionnaire developed to assess the health-related quality of life of cancer participants. The questionnaire contains 30 items and is composed of both multi-item scales and single-item measures based on the participants experience over the past week. These include five domains (physical, role, emotional, cognitive and social functioning), three symptom scales (fatigue, nausea/vomiting, and pain), six single items (dyspnea, insomnia, appetite loss, constipation, diarrhea and financial impact) and a global health status/HRQoL scale. The time to definitive 10-point deterioration is defined as the time from the date of randomization to the date of event, which is defined as at least 10 points absolute increase from baseline (worsening) of the corresponding scale score, with no later change below the threshold or death due to any cause
Change from baseline in EORTC-QLQ-C30
The EORTC QLQ-C30 is a questionnaire developed to assess the health-related quality of life of cancer participants. The questionnaire contains 30 items and is composed of both multi-item scales and single-item measures based on the participants experience over the past week. These include five domains (physical, role, emotional, cognitive and social functioning), three symptom scales (fatigue, nausea/vomiting, and pain), six single items (dyspnea, insomnia, appetite loss, constipation, diarrhea and financial impact) and a global health status/HRQoL scale. A higher score indicates a higher presence of symptoms.
Change from baseline in EORTC-QLQ-LC13
   The EORTC QLQ LC13 is a 13-item, lung cancer specific questionnaire module, and it comprises both multi-item and single-item measures of lung cancer-associated symptoms (i.e. coughing, hemoptysis, dyspnea and pain) and side-effects from conventional chemo- and radiotherapy (i.e. hair loss, neuropathy, sore mouth and dysphagia). A higher score indicates a higher presence of symptoms.
Change from baseline in EORTC-EQ-5D-5L
   The EQ-5D-5L is a generic instrument for describing and valuing health. It is based on a descriptive system that defines health in terms of 5 dimensions: Mobility, Self-Care, Usual Activities. Pain/Discomfort, and Anxiety/Depression.
Change from baseline in NSCLC-SAQ
   The Non-Small Cell Lung Cancer Symptom Assessment Questionnaire (NSCLC-SAQ) is a 7-item, patient-reported outcome measure which assess patient-reported symptoms associated with advanced NSCLC. It contains five domains and accompanying items that were identified as symptoms of NSCLC: cough (1 item), pain (2 items), dyspnea (1 item), fatigue (2 items), and appetite (1 item).
PFS based on KRAS G12C mutation status in plasma
To compare the clinical outcomes for Compound A vs docetaxel based on KRAS G12C mutation status in plasma
OS based on KRAS G12C mutation status in plasma.
To compare the clinical outcomes for Compound A vs docetaxel based on KRAS G12C mutation status in plasma
ORR based on KRAS G12C mutation status in plasma.
To compare the clinical outcomes for Compound A vs docetaxel based on KRAS G12C mutation status in plasma

### Exclusion Criteria in:

Participant has previously received docetaxel, KRAS G12C inhibitor or any other systemic therapy for their locally advanced or metastatic NSCLC other than one platinum-based chemotherapy and one prior immune check point inhibitor
Participant has EGFR-sensitizing mutation and/or ALK rearrangement by local laboratory testing
Participant has known active central nervous system (CNS) metastases and/or carcinomatous meningitis
Participant has an history of interstitial lung disease or pneumonitis grade >1. Other inclusion/exclusion criteria may apply.

### Example 15: Clinical Trials with KRAS G12C Inhibitor Such as Compound A

A KRASG12C inhibitor such as Compound A may also be investigated according to the methods described herein and in clinical trials ass described above.

For example, treatment-naive adult patients with locally advanced or metastatic NSCLC harboring KRASG12C mutations may be treated with Compound A combined with a PD1-inhiitor such as tislelizumab or with pembrolizumab combined with standard chemotherapy.

In another example, treatment-naive adult patients with locally advanced or metastatic NSCLC harboring KRASG12C mutations may be treated with Compound A combined with a PD1-inhiitor such as tislelizumab or with pembrolizumab combined with standard chemotherapy.

Efficacy of the therapeutic combinations and methods of the invention may be determined by methods well known in the art, e.g. determining Best Overall Response (BOR), Overall Response Rate (ORR), Duration of Response (DOR), Disease Control Rate (DCR), Progression Free Survival, (PFS) and Overall Survival (OS) per RECIST v.1.1, and as described herein.

It is understood that the Examples and embodiments described herein are for illustrative purposes only and that various modifications or changes in light thereof will be suggested to persons skilled in the art and are to be included within the spirit and purview of this application and scope of the appended claims.

What is claimed is:

1. A method of treating a cancer or a tumor in a subject in need thereof, wherein the method comprises administering to the subject a therapeutically effective amount of 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, (Compound A), or a pharmaceutically acceptable salt, solvate or hydrate thereof, alone or in combination with at least one additional therapeutically active agent.

2. A method according to claim 1, wherein Compound A is administered with one additional therapeutically active agent which is a SHP2 inhibitor or a PD-1 inhibitor.

3. A method according to claim 2, wherein the additional therapeutically active agent is a SHP2 inhibitor which is selected from the group consisting of TNO155, JAB3068, JAB3312, RLY1971, SAR442720, RMC4450, BBP398, BR790, SH3809, PF0724982, ERAS601, RX-SHP2, ICP189, HBI2376, ETS001, TAS-ASTX and X-37-SHP2, or a pharmaceutically acceptable salt thereof.

4. A method according to claim 1, 2 or 3, wherein the additional therapeutically active agent is TNO155, or a pharmaceutically acceptable salt thereof.

5. A method according to claim 1 or 2, wherein the additional therapeutically active agent is a PD-1 inhibitor selected from the group consisting of spartalizumab, tislelizumab, nivolumab, pembrolizumab, pidilizumab, MEDI0680, REGN2810, TSR-042, PF-06801591, BGB-108, INCSHR1210 and AMP-224.

6. A method according to claim 4 or 5, wherein the additional therapeutically active agent is spartalizumab or tislelizumab.

7. A method according to claim 1, wherein the method comprises administering to a subject in need thereof a therapeutically effective amount of 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, (Compound A), or a pharmaceutically acceptable salt, solvate or hydrate thereof, in combination with a SHP2 inhibitor and a PD-1 inhibitor.

8. A method according to claim 7, wherein the SHP2 inhibitor is selected from the group consisting of TNO155, JAB3068, JAB3312, RLY1971, SAR442720, RMC4450, BBP398, BR790, SH3809, PF0724982, ERAS601, RX-SHP2, ICP189, HB12376, ETS001, TAS-ASTX and X-37-SHP2, or a pharmaceutically acceptable salt thereof.

9. A method according to claim 8, wherein the SHP2 inhibitor is TNO155, or a pharmaceutically acceptable salt thereof.

10. A method according to claim 7 or 8 or 9, wherein the PD-1 inhibitor is selected from the group consisting of spartalizumab, tislelizumab, nivolumab, pembrolizumab, pidilizumab, MEDI0680, REGN2810, TSR-042, PF-06801591, BGB-108, INCSHR1210 and AMP-224.

11. A method according to claim 10, wherein the PD1-inhibitor is spartalizumab.

12. A method according to claim 10, wherein the PD1-inhibitor is tislelizumab.

13. A method according to any one of the previous claims, wherein the cancer or tumor is a cancer or tumor which is selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor.

14. A method according to any one of the previous claims, wherein the cancer is selected from lung cancer (such as non-small cell lung cancer), colorectal cancer, pancreatic cancer and a solid tumor.

15. A method according to any one of the previous claims wherein the cancer or tumor is a KRAS G12C mutated cancer or tumor.

16. A method according to any one of the previous claims, wherein the therapeutic agents in the combination therapy are administered simultaneously, separately or over a period of time.

17. A method according to any one of the previous claims, wherein the amount of each therapeutic agent is administered to the subject in need thereof is effective to treat the cancer or tumor.

18. A method according to any one of claims 3, 4, 8, 9, 10, 13 to 17, wherein the SHP2 inhibitor is TNO155, or pharmaceutically acceptable salt thereof, and is administered orally at a total daily dose ranging from 10 to 80 mg, or from 10 to 60 mg.

19. A method according to claim 18, wherein the dose per day of TNO155 is administered on a 21 day cycle of 2 weeks on drug followed by 1 week off drug.

20. A method according to any one of claims 5, 6, 10, 12, 13 to 17, wherein the PD1 inhibitor is PDR001 and is administered at a dose of about 300 mg once every 3 weeks.

21. A method according to any one of claims 5, 6, 10, 11, 13 to 17, wherein the PD1 inhibitor is PDR001 and is administered at a dose of about 400 mg once every 4 weeks.

22. A method according to any one of claims 5, 6, 10, 12, 13 to 17, wherein the PD1 inhibitor is tislelizumab and is administered at a dose of about 200 mg once every 3 weeks.

23. A method according to any one of claims 5, 6, 10, 12, 13 to 17, wherein the PD1 inhibitor is tislelizumab and is administered at a dose of about 300 mg once every 4 weeks.

24. A method according to any one of the previous claims, wherein Compound A, or a pharmaceutically acceptable salt, solvate or hydrate thereof, is administered at a therapeutically effective dose ranging from 50 mg to 1600 mg per day, e.g. from 200 to 1600 mg per day, e.g. from 400 to 1600 mg per day.

25. A method according to any one of the previous claims, wherein Compound A, or a pharmaceutically acceptable salt, solvate or hydrate thereof, is administered at a therapeutically effective dose which is selected from 50, 100, 150, 200, 250, 300, 350, 400, 450, 500, 550 and 600 mg per day.

26. A method according to any one of the previous claims, wherein the total daily dose of Compound A is administered once daily or twice daily.

27. A method according to any one of the previous claims, wherein the subject or patient to be treated is selected from:
a patient suffering from a KRAS G12C mutated solid tumor (e.g. advanced (metastatic or unresectable) KRAS G12C mutated solid tumor), optionally wherein the patient has received and failed standard of care therapy or is intolerant or ineligible or refractive to previous investigative and/or approved therapies;
a patient suffering from KRAS G12C mutated NSCLC (e.g., advanced (metastatic or unresectable) KRAS G12C mutated NSCLC), optionally wherein the patient who has received and failed a platinum-based chemotherapy regimen and an immune checkpoint inhibitor therapy either in combination or in sequence;
a patient suffering from KRAS G12C mutated CRC (e.g., advanced (metastatic or unresectable) KRAS G12C mutated CRC), optionally wherein the patient has received and failed standard of care therapy, including a fluropyrimidine-, oxaliplatin-, and/or irinotecan-based chemotherapy; and
a patient suffering from KRAS G12C mutated NSCLC (e.g., advanced (metastatic or unresectable) KRAS G12C mutated NSCLC), optionally wherein the patient who has previously been treated with a KRAS G12C inhibitor (e.g. sotorasib, adagrasib, GDC6036 or D-1553);
a patient suffering from KRAS G12C mutated cancer, optionally wherein the patient has previously been treated with another KRAS G12C inhibitor (e.g. sotorasib, adagrasib, GDC6036 or D-1553);
a patient suffering from a KRAS G12C mutated cancer, optionally wherein the patient has previously been treated with another KRAS G12C inhibitor (e.g. sotorasib, adagrasib, GDC6036 or D-1553) and the patient is suffering from KRAS G12C mutated cancer selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, optionally wherein the patient has previously been treated with a KRAS G12C inhibitor (e.g. sotorasib, adagrasib, GDC6036 or D-1553);
a patient suffering from lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor;
a patient suffering from a KRAS G/2C mutated cancer selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, optionally wherein the patient has previously been treated with a KRAS G12C inhibitor (e.g. sotorasib, adagrasib, GDC6036 or D-1553);
a patient suffering from lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor.

28. A pharmaceutical combination comprising
(i) 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one, having the structure 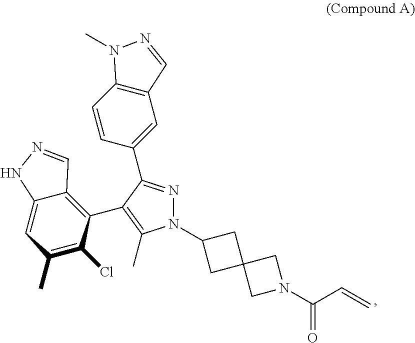
or a pharmaceutically acceptable salt, solvate or hydrate thereof,
and a SHP2 inhibitor, optionally wherein the SHP2 inhibitor is selected from TNO155, JAB3068, JAB3312, RLY1971, SAR442720, RMC4450, BBP398, BR790, SH3809, PF0724982, ERAS601, RX-SHP2, ICP189, HBI2376, ETS001, TAS-ASTX and X-37-SHP2, or a pharmaceutically acceptable salt thereof.

29. A pharmaceutical combination according to claim 28, wherein the SHP2 inhibitor is (3S,4S)-8-(6-amino-5-((2-amino-3-chloropyridin-4-yl)thio)pyrazin-2-yl)-3-methyl-2-oxa-8-azaspiro[4.5]decan-4-amine (TNO155), having the structure: 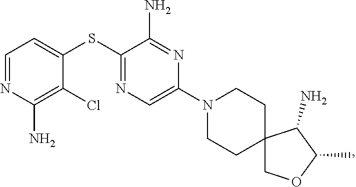 [SMILES: C[C@@H]1OCC2(CCN(c3cnc(Sc4ccnc(N)c4Cl)c(N)n3)CC2)[C@@H]1N]
or a pharmaceutically acceptable salt thereof.

30. A pharmaceutical combination comprising:
1-6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl)prop-2-en-1-one (Compound A), or a pharmaceutically acceptable salt, solvate or hydrate thereof, and
(ii) a PD-1 inhibitor.

31. A pharmaceutical combination according to claim 30, wherein the PD1-inhibitor is spartalizumab or tislelizumab.

32. A pharmaceutical combination comprising
(i) 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt, solvate or hydrate thereof,
(ii) a SHP2 inhibitor
and
(iii) a PD-1 inhibitor.

33. A pharmaceutical combination according to claim 32, wherein the SHP2 inhibitor is selected from TNO155, JAB3068, JAB3312, RLY1971, SAR442720, RMC4450, BBP398, BR790, SH3809, PF0724982, ERAS601, RX-SHP2, ICP189, HBI2376, ETS001, TAS-ASTX and X-37-SHP2, or a pharmaceutically acceptable salt thereof.

34. A pharmaceutical combination according to claim 32 or 33, wherein the PD-1 inhibitor is selected from spartalizumab, tislelizumab, nivolumab, pembrolizumab, pidilizumab, MEDI0680, REGN2810, TSR-042, PF-06801591, BGB-108, INCSHR1210 and AMP-224 (preferably from spartalizumab and tislelizumab).

35. A pharmaceutical combination according to any one of claims 28 to 34 for use in a method of treating a cancer or a solid tumor, wherein the method is according to any one of claims 1 to 27.

36. A compound which is 1-{6-[(4M)-4-(5-Chloro-6-methyl-1H-indazol-4-yl)-5-methyl-3-(1-methyl-1H-indazol-5-yl)-1H-pyrazol-1-yl]-2-azaspiro[3.3]heptan-2-yl}prop-2-en-1-one (Compound A), or a pharmaceutically acceptable salt, solvate or hydrate thereof, for use in a method of treating a cancer or a tumor, optionally wherein the cancer is selected from non-small cell lung cancer, colorectal cancer, pancreatic cancer and a solid tumor.

37. A compound for use according to claim 36, wherein the compound is administered in combination with one or two additional therapeutically active agents.

38. A compound for use according to claim 37, wherein the one or two additional therapeutically active agents is selected from TNO155, or a pharmaceutically acceptable salt thereof, and
(iii) a PD1-inhibitor such as spartalizumab or tislelizumab.

39. A compound for use according to any one of claims 36 to 38 for use in a method of treating a cancer or a solid tumor, wherein the method is according to any one of claims 1 to 27.

40. A method of treating a cancer or a tumor in a subject in need thereof, wherein the method comprises administering to the subject a therapeutically effective amount of a KRASG12C inhibitor, alone or in combination with at least one additional therapeutically active agent, wherein the subject or patient to be treated is selected from:
a patient suffering from a KRAS G12C mutated solid tumor (e.g. advanced (metastatic or unresectable) KRAS G12C mutated solid tumor), optionally wherein the patient has received and failed standard of care therapy or is intolerant or ineligible or refractive to previous investigative and/or approved therapies;
a patient suffering from KRAS G12C mutated NSCLC (e.g., advanced (metastatic or unresectable) KRAS G12C mutated NSCLC), optionally wherein the patient who has received and failed a platinum-based chemotherapy regimen and an immune checkpoint inhibitor therapy either in combination or in sequence;
a patient suffering from KRAS G12C mutated CRC (e.g., advanced (metastatic or unresectable) KRAS G12C mutated CRC), optionally wherein the patient has received and failed standard of care therapy, including a fluropyrimidine-, oxaliplatin-, and/or irinotecan-based chemotherapy; and
a patient suffering from KRAS G/2C mutated NSCLC (e.g., advanced (metastatic or unresectable) KRAS G12C mutated NSCLC), optionally wherein the patient who has previously been treated with a KRAS G12C inhibitor (e.g. sotorasib, adagrasib, GDC6036 or D-1553);
a patient suffering from KRASG12C mutated cancer, optionally wherein the patient has previously been treated with another KRAS G12C inhibitor (e.g. sotorasib, adagrasib, GDC6036 or D-1553);
a patient suffering from a KRAS G12C mutated cancer, optionally wherein the patient has previously been treated with another KRAS G12C inhibitor (e.g. sotorasib, adagrasib, GDC6036 or D-1553) and the patient is suffering from KRAS G12C mutated cancer selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, optionally wherein the patient has previously been treated with a KRAS G12C inhibitor (e.g. sotorasib, adagrasib, GDC6036 or D-1553);
a patient suffering from lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor;
a patient suffering from a KRAS G12C mutated cancer selected from the group consisting of lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor, optionally wherein the patient has previously been treated with a KRAS G12C inhibitor (e.g. sotorasib, adagrasib, GDC6036 or D-1553);
a patient suffering from lung cancer (including lung adenocarcinoma, non-small cell lung cancer and squamous cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma), appendiceal cancer, small-bowel cancer, esophageal cancer, hepatobiliary cancer (including liver cancer and bile duct carcinoma), bladder cancer, ovarian cancer and a solid tumor;
a treatment-naive patient with locally advanced or metastatic NSCLC harboring KRASG12C mutations.

41. A method according to claim 40, wherein the at least additional therapeutically active agent is selected from a SHP2 inhibitor (such as TNO 155) and a PD1-inhibitor (such as tislelizumab).


SEQUENCE LISTING (563 sequences)

SEQ ID NO: 1-500 - skipped (500 entries)

SEQ ID NO: 501 - protein, 5 aa, Artificial Sequence
  Synthetic polypeptide
TYWMH

SEQ ID NO: 502 - protein, 17 aa, Artificial Sequence
  Synthetic polypeptide
NIYPGTGGSNFDEKFKN

SEQ ID NO: 503 - protein, 8 aa, Artificial Sequence
  Synthetic polypeptide
WTTGTGAY

SEQ ID NO: 504 - protein, 7 aa, Artificial Sequence
  Synthetic polypeptide
GYTFTTY

SEQ ID NO: 505 - protein, 6 aa, Artificial Sequence
  Synthetic polypeptide
YPGTGG

SEQ ID NO: 506 - protein, 117 aa, Artificial Sequence
  Synthetic polypeptide
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, Artificial Sequence
  Synthetic polynucleotide
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, Artificial Sequence
  Synthetic polypeptide
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, Artificial Sequence
  Synthetic polynucleotide
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, Artificial Sequence
  Synthetic polypeptide
KSSQSLLDSGNQKNFLT

SEQ ID NO: 511 - protein, 7 aa, Artificial Sequence
  Synthetic polypeptide
WASTRES

SEQ ID NO: 512 - protein, 9 aa, Artificial Sequence
  Synthetic polypeptide
QNDYSYPYT

SEQ ID NO: 513 - protein, 13 aa, Artificial Sequence
  Synthetic polypeptide
SQSLLDSGNQKNF

SEQ ID NO: 514 - protein, 3 aa, Artificial Sequence
  Synthetic polypeptide
WAS

SEQ ID NO: 515 - protein, 6 aa, Artificial Sequence
  Synthetic polypeptide
DYSYPY

SEQ ID NO: 516 - protein, 113 aa, Artificial Sequence
  Synthetic polypeptide
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, Artificial Sequence
  Synthetic polynucleotide
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, Artificial Sequence
  Synthetic polypeptide
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, Artificial Sequence
  Synthetic polynucleotide
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, Artificial Sequence
  Synthetic polypeptide
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, Artificial Sequence
  Synthetic polynucleotide
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, Artificial Sequence
  Synthetic polypeptide
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, Artificial Sequence
  Synthetic polynucleotide
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, Artificial Sequence
  Synthetic polynucleotide
acctactggatgcac

SEQ ID NO: 525 - DNA, 51 bp, Artificial Sequence
  Synthetic polynucleotide
aatatctaccccggcaccggcggctctaacttcgacgagaagtttaagaat

SEQ ID NO: 526 - DNA, 24 bp, Artificial Sequence
  Synthetic polynucleotide
tggactaccggcacaggcgcctac

SEQ ID NO: 527 - DNA, 21 bp, Artificial Sequence
  Synthetic polynucleotide
ggctacaccttcactacctac

SEQ ID NO: 528 - DNA, 18 bp, Artificial Sequence
  Synthetic polynucleotide
taccccggcaccggcggc

SEQ ID NO: 529 - DNA, 51 bp, Artificial Sequence
  Synthetic polynucleotide
aaatctagtcagtcactgctggatagcggtaatcagaagaacttcctgacc

SEQ ID NO: 530 - DNA, 21 bp, Artificial Sequence
  Synthetic polynucleotide
tgggcctctactagagaatca

SEQ ID NO: 531 - DNA, 27 bp, Artificial Sequence
  Synthetic polynucleotide
cagaacgactatagctacccctacacc

SEQ ID NO: 532 - DNA, 39 bp, Artificial Sequence
  Synthetic polynucleotide
agtcagtcactgctggatagcggtaatcagaagaacttc

SEQ ID NO: 533 - DNA, 9 bp, Artificial Sequence
  Synthetic polynucleotide
tgggcctct

SEQ ID NO: 534 - DNA, 18 bp, Artificial Sequence
  Synthetic polynucleotide
gactatagctacccctac

SEQ ID NO: 535-541 - skipped (7 entries)

SEQ ID NO: 542 - protein, 11 aa, Artificial Sequence
  Synthetic polypeptide
KSSESVSNDVA

SEQ ID NO: 543 - protein, 7 aa, Artificial Sequence
  Synthetic polypeptide
YAFHRFT

SEQ ID NO: 544 - protein, 9 aa, Artificial Sequence
  Synthetic polypeptide
HQAYSSPYT

SEQ ID NO: 545 - protein, 107 aa, Artificial Sequence
  Synthetic polypeptide
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 546 - protein, 214 aa, Artificial Sequence
  Synthetic polypeptide
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 547 - protein, 10 aa, Artificial Sequence
  Synthetic polypeptide
GFSLTSYGVH

SEQ ID NO: 548 - protein, 16 aa, Artificial Sequence
  Synthetic polypeptide
VIYADGSTNYNPSLKS

SEQ ID NO: 549 - protein, 12 aa, Artificial Sequence
  Synthetic polypeptide
ARAYGNYWYIDV

SEQ ID NO: 550 - protein, 118 aa, Artificial Sequence
  Synthetic polypeptide
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 551 - protein, 445 aa, Artificial Sequence
  Synthetic polypeptide
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 552 - DNA, 41 bp, Artificial Sequence
  Synthetic polynucleotide
gtcatttgaagatatccaccgttatcgcgagcagattaaga

SEQ ID NO: 553 - DNA, 41 bp, Artificial Sequence
  Synthetic polynucleotide
tcttaatctgctcgcgataacggtggatatcttcaaatgac

SEQ ID NO: 554 - DNA, 41 bp, Artificial Sequence
  Synthetic polynucleotide
tcatttgaagatatccaccagtatcgcgagcagattaagag

SEQ ID NO: 555 - DNA, 41 bp, Artificial Sequence
  Synthetic polynucleotide
ctcttaatctgctcgcgatactggtggatatcttcaaatga

SEQ ID NO: 556 - DNA, 41 bp, Artificial Sequence
  Synthetic polynucleotide
agtcatttgaagatatccacgattatcgcgagcagattaag

SEQ ID NO: 557 - DNA, 41 bp, Artificial Sequence
  Synthetic polynucleotide
cttaatctgctcgcgataatcgtggatatcttcaaatgact

SEQ ID NO: 558 - DNA, 39 bp, Artificial Sequence
  Synthetic polynucleotide
gaagagtactccgcaatgagcgatcaatacatgaggacg

SEQ ID NO: 559 - DNA, 39 bp, Artificial Sequence
  Synthetic polynucleotide
cgtcctcatgtattgatcgctcattgcggagtactcttc

SEQ ID NO: 560 - DNA, 42 bp, Artificial Sequence
  Synthetic polynucleotide
cgaagtcatttgaagatatccaccattgtcgcgagcagatta

SEQ ID NO: 561 - DNA, 42 bp, Artificial Sequence
  Synthetic polynucleotide
taatctgctcgcgacaatggtggatatcttcaaatgacttcg

SEQ ID NO: 562 - DNA, 41 bp, Artificial Sequence
  Synthetic polynucleotide
cgaagtcatttgaagatatccaccatgatcgcgagcagatt

SEQ ID NO: 563 - DNA, 41 bp, Artificial Sequence
  Synthetic polynucleotide
aatctgctcgcgatcatggtggatatcttcaaatgacttcg